SEQUENCE LISTING (42 sequences)

SEQ ID NO: 1 - protein, 10 aa, synthetic construct
  REGION 1..10: note = c-myc tag
EQKLISEEDL

SEQ ID NO: 2 - protein, 7 aa, synthetic construct
  REGION 1..7: note = 7XHIS tag
HHHHHHH

SEQ ID NO: 3 - DNA, 459 bp, Homo sapiens
  source 1..459: mol_type = genomic DNA
atgtcttgttctttacctatggctagatactacattattaaatacgctgatcaaaaagccttatacactagagatggacaattattagtaggagatcctgtagctgataattgttgtgctgaaaaaatttgtattttacctaatagaggattagctagaactaaagtacctattttcttaggaattcaaggaggatctagatgtttagcttgtgtagaaactgaagaaggaccttctttacaattagaagatgtaaatattgaagaattatacaaaggaggagaagaagctactagattcactttcttccaatcttcttctggatctgctttcagattagaagctgctgcttggcctggatggttcttatgtggaccagctgaacctcaacaacctgtacaattaactaaagaatctgaaccttctgctagaactaaattctacttcgaacaatcttgg

SEQ ID NO: 4 - DNA, 537 bp, Homo sapiens
  source 1..537: mol_type = genomic DNA
atgcctggacctcctcctggacctcctagagtatctcctgatcctagagctgaattagattctactgtattattaactagatctttattagctgatactagacaattagctgctcaattaagagataaattccctgctgatggagatcataatttagattctttacctactttagctatgtctgctggagctttaggagctttacaattacctggagtattaactagattaagagctgatttattatcttacttaagacatgtacaatggttaagaagagctggaggatcttctttaaaaactttagaacctgaattaggaactttacaagctagattagatagattattaagaagattacaattattaatgtctagattagctttacctcaacctcctcctgatcctcctgctcctcctttagctcctccttcttctgcttggggaggaattagagctgctcatgctattttaggaggattacatttaactttagattgggctgtaagaggattattattattaaaaactagatta

SEQ ID NO: 5 - DNA, 522 bp, Homo sapiens
  source 1..522: mol_type = genomic DNA
atggtacatacttctcctaaagtaaaaaatttaaatcctaaaaaattctctattcatgatcaagatcataaagtattagtattagattctggaaatttaattgctgtacctgataaaaattacattagacctgaaattttcttcgctttagcttcttctttatcttctgcttctgctgaaaaaggatctcctattttattaggagtatctaaaggagaattctgtttatactgtgataaagataaaggacaatctcatccttctttacaattaaaaaaagaaaaattaatgaaattagctgctcaaaaagaatctgctagaagacctttcattttctacagagctcaagtaggatcttggaatatgttagaatctgctgctcatcctggatggttcatttgtacttcttgtaattgtaatgaacctgtaggagtaactgataaattcgaaaatagaaaacatattgaattctctttccaacctgtatgtaaagctgaaatgtctccttctgaagtatctgat

SEQ ID NO: 6 - DNA, 684 bp, Homo sapiens
  source 1..684: mol_type = genomic DNA
atgaaacctaaaatgaaatactctactaataaaatttctactgctaaatggaaaaatactgcttctaaagctttatgtttcaaattaggaaaatctcaacaaaaagctaaagaagtatgtcctatgtacttcatgaaattaagatctggattaatgattaaaaaagaggcttgttacttcagaagagaaactactaaaagaccttctttaaaaactggaatttctcctattactgaatacttagcttctttatctacttacaatgatcaatctattactttcgctttagaagatgaatcttacgaaatttacgtagaagatttaaaaaaagatgaaaaaaaagataaagtattattatcttactacgaatctcaacatccttctaatgaatctggagatggagtagatggaaaaatgttaatggtaactttatctcctactaaagatttctggttacatgctaataataaagaacattctgtagaattacataaatgtgaaaaacctttacctgatcaagcattcttcgtattacataatatgcattctaattgtgtatctttcgaatgtaaaactgatcctggagtattcattggagtaaaagataatcatttagctttaattaaagtagattcttctgaaaatttatgtactgaaaatattttattcaaattatctgaaact

SEQ ID NO: 7 - DNA, 459 bp, Homo sapiens
  source 1..459: mol_type = genomic DNA
atgagaccttctggaagaaaatcttctaaaatgcaagcattcagaatttgggatgtaaatcaaaaaactttctacttaagaaataatcaattagtagctggatacttacaaggacctaatgtaaatttagaagaaaaaattgatgtagtacctattgaacctcatgctttattcttaggaattcatggaggaaaaatgtgtttatcttgtgtaaaatctggagatgaaactagattacaattagaagctgtaaatattactgatttatctgaaaatagaaaacaagataaaagattcgctttcattagatctgattctggacctactacttctttcgaatctgctgcttgtcctggatggttcttatgtactgctatggaagctgatcaacctgtatctttaactaatatgcctgatgaaggagtaatggtaactaaattctacttccaagaagatgaa

SEQ ID NO: 8 - DNA, 465 bp, Homo sapiens
  source 1..465: mol_type = genomic DNA
atggtattatctggagctttatgtttcagaatgaaagattctgctttaaaagtattatacttacataataatcaattattagctggaggattacatgaaggaaaagtaattaaaggagaagaaatttctgtagtacctaatagatggttagatgcttctttatctcctgtaattttaggagtacaaggaggatctcaatgtttatcttgtggagtaggacaagaacctactttaactttagaacctgtaaatattatggaattatacttagtagctaaagaatctaaatctttcactttctacagaagagatatgggattaacttcttctttcggatctgctgcttaccctggatggttcttatgtactgtacctgaagctgatcaacctgtaagattaactcaattacctgaaaatggaggatggaatgctcctattactgatttctacttccaacaatgtgat

SEQ ID NO: 9 - DNA, 402 bp, Homo sapiens
  source 1..402: mol_type = genomic DNA
atggctcctacttcttcttctactaaaaaaactcaattacaattagaacatttattattagatttacaaatgattttaaatggaattaataattacaaaaatcctaaattaactagaatgttaactttcaaattctacatgcctaaaaaagctactgaattaaaacatttacaatgtttagaagaagaattaaaacctttagaagaagtattaaatttagctcaatctaaaaatttccatttaagacctagagatttaatttctaatattaatgtaattgtattagaattaaaaggatctgaaactactttcatgtgtgaatacgctgatgaaactgctactattgtagaattcttaaatagatggattactttctgtcaatctattatttctactttaact

SEQ ID NO: 10 - DNA, 402 bp, Homo sapiens
  source 1..402: mol_type = genomic DNA
atggctcctatgactcaaactactcctttaaaaacttcttgggtaaattgttctaatatgattgatgaaattattactcatttaaaacaacctcctttacctttattagatttcaataatttaaatggagaagatcaagatattttaatggaaaataatttaagaagacctaatttagaagcattcaatagagctgtaaaatctttacaaaatgcttctgctattgaatctattttaaaaaatttattaccttgtttacctttagctactgctgctcctactagacatcctattcatattaaagatggagattggaatgaattcagaagaaaattaactttctacttaaaaactttagaaaatgctcaagctcaacaaactactttatctttagctattttc

SEQ ID NO: 11 - DNA, 465 bp, Homo sapiens; identical to SEQ ID NO: 8
  source 1..465: mol_type = genomic DNA

SEQ ID NO: 12 - DNA, 537 bp, Homo sapiens
  source 1..537: mol_type = genomic DNA
atggctactcctttaggaccagcttcttctttacctcaatctttcttattaaaatgtttagaacaagtaagaaaaattcaaggagatggagctgctttacaagaaaaattagtatctgaatgtgctacttacaaattatgtcatcctgaagaattagtattattaggacattctttaggaattccttgggctcctttatcttcttgtccttctcaagcattacaattagctggatgtttatctcaattacattctggattattcttataccaaggattattacaagccttagaaggaatttctcctgaattaggacctactttagatactttacaattagatgtagctgatttcgctactactatttggcaacaaatggaagaattaggaatggctcctgctttacaacctactcaaggagctatgcctgctttcgcttctgctttccaaagaagagctggaggagtattagtagcttctcatttacaatctttcttagaagtatcttacagagtattaagacatttagctcaacct

SEQ ID NO: 13 - DNA, 339 bp, Homo sapiens
  source 1..339: mol_type = genomic DNA
atgggacctgtacctccttctactgctttaagagaattaattgaagaattagtaaatattactcaaaatcaaaaagctcctttatgtaatggatctatggtatggtctattaatttaactgctggaatgtactgtgctgctttagaatctttaattaatgtatctggatgttctgctattgaaaaaactcaaagaatgttatctggattctgtcctcataaagtatctgctggacaattctcttctttacatgtaagagatactaaaattgaagtagctcaattcgtaaaagatttattattacatttaaaaaaattattcagagaaggaagattcaat

SEQ ID NO: 14 - DNA, 579 bp, Homo sapiens
  source 1..579: mol_type = genomic DNA
atgttagctttctctgatgctggacctcatgtacattacggatggggagatcctattagattaagacatttatacacttctggacctcatggattatcttcttgtttcttaagaattagagctgatggagtagtagattgtgctagaggacaatctgctcattctttattagaaattaaagctgtagctttaagaactgtagctattaaaggagtacattctgtaagatacttatgtatgggagctgatggaaaaatgcaaggattattacaatactctgaagaagattgtgctttcgaagaagaaattagacctgatggatacaatgtatacagatctgaaaaacatagattacctgtatctttatcttctgctaaacaaagacaattatacaaaaatagaggattcttacctttatctcatttcttacctatgttacctatggtacctgaagaacctgaagatttaagaggacatttagaatctgatatgttctcttctcctttagaaactgattctatggatcctttcggattagtaactggattagaagctgtaagatctccttctttcgaaaaa

SEQ ID NO: 15 - DNA, 384 bp, Homo sapiens
  source 1..384: mol_type = genomic DNA
atggctcctgctagatctccttctccttctactcaaccttgggaacatgtaaatgctattcaagaagctagaagattattaaatttatctagagatactgctgctgaaatgaatgaaactgtagaagtaatttctgaaatgttcgatttacaagaacctacttgtttacaaactagattagaattatacaaacaaggattaagaggatctttaactaaattaaaaggacctttaactatgatggcttctcattacaaacaacattgtcctcctactcctgaaacttcttgtgctactcaaattattactttcgaatctttcaaagaaaatttaaaagatttcttattagtaattcctttcgattgttgggaacctgtacaagaa

SEQ ID NO: 16 - DNA, 1269 bp, Homo sapiens
  source 1..1269: mol_type = genomic DNA
atgagaaaaggacctcctgctgctttaactttacctagagtacaatgtagagcttctagataccctattgctgtagattgttcttggactttacctcctgctcctaattctacttctcctgtatctttcattgctacttacagattaggaatggctgctagaggacattcttggccttgtttacaacaaactcctacttctacttcttgtactattactgatgtacaattattctctatggctccttacgtattaaatgtaactgctgtacatccttggggatcttcttcttctttcgtacctttcattactgaacatattattaaacctgatcctcctgaaggagtaagattatctcctttagctgaaagacaattacaagtacaatgggaacctcctggatcttggcctttccctgaaattttctctttaaaatactggattagatacaaaagacaaggagctgctagattccatagagtaggacctattgaagctacttctttcattttaagagctgtaagacctagagctagatactacgtacaagtagctgctcaagatttaactgattacggagaattatctgattggtctttaccagctactgctactatgtctttaggaaaatctggaggaggaggatctggaggaggaggatctggaggaggaggatctagaaatttacctgtagctactcctgatcctggaatgttcccttgtttacatcattctcaaaatttattaagagctgtatctaatatgttacaaaaagctagacaaactttagaattctacccttgtacttctgaagaaattgatcatgaagatattactaaagataaaacttctactgtagaagcatgtttacctttagaattaactaaaaatgaatcttgtttaaattctagagaaacttctttcattactaatggatcttgtttagcttctagaaaaacttctttcatgatggctttatgtttatcttctatttacgaagatttaaaaatgtaccaagtagaattcaaaactatgaatgctaaattattaatggatcctaaaagacaaattttcttagatcaaaatatgttagctgtaattgatgaattaatgcaagcattaaatttcaattctgaaactgtacctcaaaaatcttctttagaagaacctgatttctacaaaactaaaattaaattatgtattttattacatgctttcagaattagagctgtaactattgatagagtaatgtcttacttaaatgcttct

SEQ ID NO: 17 - DNA, 465 bp, Homo sapiens; identical to SEQ ID NO: 8
  source 1..465: mol_type = genomic DNA

SEQ ID NO: 18 - DNA, 390 bp, Homo sapiens
  source 1..390: mol_type = genomic DNA
atgcataaatgtgatattactttacaagaaattattaaaactttaaattctttaactgaacaaaaaactttatgtactgaattaactgtaactgatattttcgctgcttctaaaaatactactgaaaaagaaactttctgtagagctgctactgtattaagacaattctactctcatcatgaaaaagatactagatgtttaggagctactgctcaacaattccatagacataaacaattaattagattcttaaaaagattagatagaaatttatggggattagctggattaaattcttgtcctgtaaaagaagctaatcaatctactttagaaaatttcttagaaagattaaaaactattatgagagaaaaatactctaaatgttcttct

SEQ ID NO: 19 - DNA, 345 bp, Homo sapiens
  source 1..345: mol_type = genomic DNA
atgcaagctaaacataaacaaagaaaaagattaaaatcttcttgtaaaagacatcctttatacgtagatttctctgatgtaggatggaatgattggattgtagctcctcctggataccatgctttctactgtcatggagaatgtcctttccctttagctgatcatttaaattctactaatcatgctattgtacaaactttagtaaattctgtaaattctaaaattcctaaagcatgttgtgtacctactgaattatctgctatttctatgttatacttagatgaaaatgaaaaagtagtattaaaaaattaccaagatatggtagtagaaggatgtggatgtaga

SEQ ID NO: 20 - DNA, 420 bp, Homo sapiens
  source 1..420: mol_type = genomic DNA
atgtctactggatctaaacaaagatctcaaaatagatctaaaactcctaaaaatcaagaggctttaagaatggctaatgtagctgaaaattcttcttctgatcaaagacaggcttgtaaaaaacatgaattatacgtatctttcagagatttaggatggcaagattggattattgctcctgaaggatacgctgcttactactgtgaaggagaatgtgctttccctttaaattcttacatgaatgctactaatcatgctattgtacaaactttagtacatttcattaatcctgaaactgtacctaaaccttgttgtgctcctactcaattaaatgctatttctgtattatacttcgatgattcttctaatgtaattttaaaaaaatacagaaatatggtagtaagagcttgtggatgtcat

SEQ ID NO: 21 - DNA, 339 bp, Homo sapiens
  source 1..339: mol_type = genomic DNA
atggctttagatactaattactgtttctcttctactgaaaaaaattgttgtgtaagacaattatacattgatttcagaaaagatttaggatggaaatggattcatgaacctaaaggataccatgctaatttctgtttaggaccttgtccttacatttggtctttagatactcaatactctaaagtattagctttatacaatcaacataatcctggagcttctgctgctccttgttgtgtacctcaagctttagaacctttacctattgtatactacgtaggaagaaaacctaaagtagaacaattatctaatatgattgtaagatcttgtaaatgttct

SEQ ID NO: 22 - DNA, 657 bp, Homo sapiens
  source 1..657: mol_type = genomic DNA
atgaattcttgtgaattaactaatattactattgctattgaaaaagaagaatgtagattctgtatttctattaatactacttggtgtgctggatactgttacactagagatttagtatacaaagatcctgctagacctaaaattcaaaaaacttgtactttcaaagaattagtatacgaaactgtaagagtacctggatgtgctcatcatgctgattctttatacacttaccctgtagctactcaatgtcattgtggaaaatgtgattctgattctactgattgtactgtaagaggattaggaccttcttactgttctttcggagaaatgaaagaaggaggaggatctggaggaggatctggaggaggatctggaggaggagctcctgatgtacaagattgtcctgaatgtactttacaagaaaatcctttcttctctcaacctggagctcctattttacaatgtatgggatgttgtttctctagagcttaccctactcctttaagatctaaaaaaactatgttagtacaaaaaaatgtaacttctgaatctacttgttgtgtagctaaatcttacaatagagtaactgtaatgggaggattcaaagtagaaaatcatactgcttgtcattgttctacttgttactaccataaatct

SEQ ID NO: 23 - DNA, 73 bp, Nicotiana tabacum
  source 1..73: mol_type = genomic DNA
ttagcatactgactcgctttcatccttcccgttcatagaccaagggaaactctttttagtaagtgttagtgtt

SEQ ID NO: 24 - DNA, 66 bp, Nicotiana tabacum
  source 1..66: mol_type = genomic DNA
tctatgataggatcgtttatttacaacggaatggtatacaaagtcaacagatctcaatgaatacaa

SEQ ID NO: 25 - DNA, 73 bp, Nicotiana tabacum
  source 1..73: mol_type = genomic DNA
agtattctaaatcttagttggtattcaaaatatccgattcaagtagacaaagagatggttgaatcaaaaaatt

SEQ ID NO: 26 - DNA, 73 bp, Nicotiana tabacum
  source 1..73: mol_type = genomic DNA
cagttcagggttcctcgtctctttttttttttttgaaaaagaataaaaaaaaaaggggggggtgtggagagaa

SEQ ID NO: 27 - DNA, 78 bp, Nicotiana tabacum
  source 1..78: mol_type = genomic DNA
cttccattttctattttgatttgtagaaaactagtgtgcttgggagtccctgatgattaaataaaccaagattttacc

SEQ ID NO: 28 - DNA, 85 bp, Teseptimavirus T7
  source 1..85: mol_type = genomic DNA
gaaattaatacgactcactatagggagaccacaacggtttccctctagaaataattttgtttaactttaagaaggagatatacat

SEQ ID NO: 29 - DNA, 57 bp, Nicotiana tabacum
  source 1..57: mol_type = genomic DNA
tcgagtagaccttgttgttgtgagaattcttaattcatgagttgtagggagggattt

SEQ ID NO: 30 - DNA, 178 bp, Populus alba
  source 1..178: mol_type = genomic DNA
ttgagaaaagagatccaatagttgaagtaggaatccatttgattctactatacatattgattgggtcgatcaggtcatacttagagagtctgcctttttcctttcttttcaactcatttagatttagatctaatcgatttttttatggctcggctatcctacctagtcgagccattca

SEQ ID NO: 31 - DNA, 233 bp, Populus alba
  source 1..233: mol_type = genomic DNA
tccactaattaatgttcattctcgtaattgaattgcaattaaattaaactcggcccaatcttttactaaaaggattgagccgaatacaaatagcctattgtatatatttgtatatatttttgatagatacatacttagctaaatatacaagatcttaaatacaaaatataagactcaaagaaatcaaacctttctactcttgtcttagatccctaattaatcctaaggatgtg

SEQ ID NO: 32 - DNA, 168 bp, Medicago truncatula
  source 1..168: mol_type = genomic DNA
gattttggttttcaaaaaggatacgcgtttttgaaaataaaggggtaaaggagtaatatcaacattgttgatattactcccccttttactttttgttagtagtctttttctgtatgcaatacatatacagaaattaatcaattatttattaagttcatttagcatttt

SEQ ID NO: 33 - DNA, 320 bp, Escherichia coli
  source 1..320: mol_type = genomic DNA
ggatcctcaactgtgaggaggctcacggacgcgaagaacaggcacgcgtacaggaaacacagaaaaaagcccgcacctgacagtgcgggctttttttttcgaccaaaggtaacgaggtaacaaccatgcgagtgttgaagttcggcggtacatcagtggcaaatgcagaacgttttctgcgtgttgccgatattctggaaagcaatgccaggcaggggcaggtgggggaattcactggccgtcgttttacaacgtcgtgactgggaaaaccctggcgttacccaacttaatcgccttgcagcacatccccctttcgccag

SEQ ID NO: 34 - DNA, 62 bp, Nicotiana tabacum
  source 1..62: mol_type = genomic DNA
gatctacatacaccttggttgacacgagtatataagtcatgttatactgttgaataacaagc

SEQ ID NO: 35 - DNA, 83 bp, Synthetic construct
  source 1..83: mol_type = other DNA
  misc_feature 1..83: note = Core promoter of the plastid rrn operon of the Nicotiana tobacum plastome engineered with two triplet nucleotide mutations
gctcccccgccgtcgttcaatgagatacgataagaggctcgtgggattgacgtgagggggctccgatggctatatttctggga

SEQ ID NO: 36 - DNA, 41 bp, Nicotiana tabacum
  source 1..41: mol_type = genomic DNA
attgggttgcgctatatatatgaaagagtatacaataatga

SEQ ID NO: 37 - DNA, 795 bp, synthetic construct
  misc_feature 1..795: note = selectable marker gene aadA
  source 1..795: mol_type = other DNA
atggctcgtgaagcggttatcgccgaagtatcaactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgctggaatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataa

SEQ ID NO: 38 - DNA, 825 bp, synthetic construct
  misc_feature 1..825: note = rGFP gene with HIS-tag and c-myc tag
  source 1..825: mol_type = other DNA
atggctagcaggtctcatcatcatcatcatcatcatgataataaacaagaaaatttatacttccaaggaagaaaaggagaagaattattcactggagtagtacctattttagtagaattagatggagatgtaaatggacataaattctctgtaagaggagaaggagaaggagatgctactaatggaaaattaactttaaaattcatttgtactactggaaaattacctgtaccttggcctactttagtaactactttaacttacggagtacaatgtttcgctagataccctgatcatatgaaacaacatgatttcttcaaatctgctatgcctgaaggatacgtacaagaaagaactatttctttcaaagatgatggaacttacaaaactagagctgaagtaaaattcgaaggagatactttagtaaatagaattgaattaaaaggaattgatttcaaagaagatggaaatattttaggacataaattagaatacaatttcaattctcataatgtatacattactgctgataaacaaaaaaatggaattaaagctaatttcaaaattagacataatgtagaagatggatctgtacaattagctgatcattaccaacaaaatactcctattggagatggacctgtattattacctgataatcattacttatctactcaatctgtattatctaaagatcctaatgaaaaaagagatcatatggtattattagaattcgtaactgctgctggaattactcatggaatggatgaattatacaaagaacctgctcttgaacaaaaattaatttctgaagaagatttataa

SEQ ID NO: 39 - protein, 5 aa, synthetic construct
  REGION 1..5: note = peptide linker
SGGGG

SEQ ID NO: 40 - protein, 43 aa, synthetic construct
  REGION 1..43: note = P1 from the NSP10 protein of SARS-CoV-2
YGRKKRRQRRRGSGFGGASCCLYCRCHIDHPNPKGFCDLKGKY

SEQ ID NO: 41 - protein, 43 aa, synthetic construct
  REGION 1..43: note = peptide P2 from NSP10 protein of SARS-CoV-2
YGRKKRRQRRRGSGFGGASCCLYCRCRIDHPNPKGFCDLKGKY

SEQ ID NO: 42 - protein, 14 aa, synthetic construct
  REGION 1..14: note = HIV-Tat peptide sequence
YGRKKRRQRRRGSG

(19) United States
(12) Patent Application Publication
(10) Pub. No.: US 2025/0101446 A1
(43) Pub. Date: Mar. 27, 2025

(54) METHOD FOR GENETIC TRANSFORMATION OF PLANT ORGANELLE GENOME AND USES THEREOF

(71) Applicant: Solar Grants Biotechnology Inc., London (CA)

(72) Inventor: Igor Kolotilin, London (CA)

(21) Appl. No.: 18/725,603

(22) PCT Filed: Jan. 6, 2023
(86) PCT No.: PCT/CA2023/050008

§ 371 Date: Jun. 28, 2024

Related U.S. Application Data
(60) Provisional application No. 63/297,500, filed on Jan. 7, 2022.

(51) Int. Cl. C12N 15/82; C07K 14/31; C07K 14/47; C07K 14/495; C07K 14/50; C07K 14/535; C07K 14/54; C07K 14/715

(52) U.S. Cl. CPC C12N 15/8214; C07K 14/31; C07K 14/4702; C07K 14/495; C07K 14/50; C07K 14/535; C07K 14/54; C07K 14/7155; C12N 15/8257

(57) ABSTRACT

A transformation vector for stably transforming a plastid is provided. The transformation vector comprises an expression cassette, comprising, as operably-linked components, a regulatory sequence operative in the plastid, a heterologous polynucleotide sequence coding for a protein of interest, and, flanking each side of the expression cassette, a first DNA flanking sequence and a second flanking DNA sequence which allow for stable integration of the heterologous polynucleotide sequence coding for the protein of interest into the plastid genome. Methods of producing the protein of interest and uses thereof are also provided.

### FIELD

The present disclosure relates to transformation vectors. In particular, the present disclosure relates to methods of transforming plastids in plants using transformation vectors to produce proteins of interest which are biologically active, and uses thereof.

### BACKGROUND

Research efforts have been made to synthesize high value, immunologically and pharmacologically active recombinant proteins in plants.

U.S. Publication No. 20180371485 is directed to vaccines for conferring immunity in mammals to infective pathogens, as well as to vectors and methods for plastid transformation of plants to produce protective antigens and vaccines for oral delivery.

U.S. Pat. No. 9,605,045 is directed to a plastid transformation vector for stably transforming a plastid genome, comprising, as operably linked components, a first flanking sequence, a DNA sequence coding for synthetic insulin-like growth factor-1 (IGF-1s) or a substantially homologous DNA sequence of IGF-1s, which is capable of expression in the plastid genome, and a second flanking sequence.

U.S. Publication No. 20100304476 is directed to human serum albumin (HSA) or a HSA fusion protein that is expressed in plant plastids. Plastid transformation vectors are made which contain an expression cassette that contains regulatory sequences, the coding region for HSA or an HSA fusion protein and a selectable marker coding sequence. The vector is used to transform a plant where the plant expresses the HSA or HSA fusion protein.

A need exists for the development of a product, composition and/or method that provides the public with a useful alternative.

### SUMMARY

In an aspect, there is provided a transformation vector for stably transforming a plastid, comprising, an expression cassette, comprising, as operably-linked components, a regulatory sequence operative in the plastid, a heterologous polynucleotide sequence coding for a protein of interest, and, flanking each side of the expression cassette, a first DNA flanking sequence and a second flanking DNA sequence which allow for stable integration of the heterologous polynucleotide sequence coding for the protein of interest into the plastid genome.

In aspects, the first flanking sequence comprises trnl, rps12 or tmnT.

In aspects, the first flanking sequence comprises trnl.

In aspects, the first flanking sequence comprises rps12.

In aspects, the first flanking sequence comprises trnT.

In aspects, the second flanking sequence comprises tmnA, or trnV or trnG.

In aspects, the second flanking sequence comprises trnA.

In aspects, the second flanking sequence comprises tmnV.

In aspects, the second flanking sequence comprises trnG.

In aspects, the first flanking sequence is substantially homologous to a sequence around an integration site of the plastid genome and provides for homologous recombination to insert the heterologous polynucleotide coding for the protein of interest into the integration site of the plastid genome.

In aspects, the second flanking sequences is substantially homologous to a sequence around an integration site of the plastid genome and provides for homologous recombination to insert the heterologous polynucleotide coding for the protein of interest into the integration site of the plastid genome.

In aspects, the first and second flanking sequences are substantially homologous to sequences around an integration site of the plastid genome and provide for homologous recombination to insert the heterologous polynucleotide coding for the protein of interest into the integration site of the plastid genome.

In aspects, the expression cassette further comprises a spacer region comprising about 50 to about 80 base pairs.

In aspects, the spacer is from between psbN and psbH genes, or rps2 and atpl genes, or rpoC2 and rps2 genes of the plastid genome.

In aspects, the spacer is from between psbN and psbH genes of the plastid genome.

In aspects, the spacer is from between rps2 and atpl genes of the plastid genome.

In aspects, the spacer is from between rpoC2 and rps2 genes of the plastid genome.

In aspects, the regulatory sequence comprises a promoter operative in the plastid genome.

In aspects, the promoter is 16S rRNA, psbA gene or rbcL gene.

In aspects, the promoter is 16S rRNA.

In aspects, the promoter is the psbA gene.

In aspects, the promoter is the rbcL gene.

In aspects, the promoter is a mutated 16S rRNA promoter with reduced homology to the endogenous 16S rRNA promoter yet with substantially equal functionality to that of the endogenous 16S rRNA promoter.

In aspects, the regulatory sequence further comprises a 5′ untranslated region (UTR) capable of providing transcription and translation enhancement of the heterologous polynucleotide coding for the protein of interest.

In aspects, the 5′ UTR is a 5′ UTR of psbA or T7G10.

In aspects, the 5′ UTR is a 5′ UTR of psbA.

In aspects, the 5′ UTR is a 5′ UTR of T7G10.

In aspects, the regulatory sequence further comprises a 3′ UTR capable of conferring stability to a transcript of the protein of interest.

In aspects, the 3′ UTR is a 3′ UTR of psbA or a heterologous psbC gene.

In aspects, the 3′ UTR is a 3′ UTR of psbA.

In aspects, the 3′ UTR is a 3′ UTR of a heterologous psbC gene.

In aspects, the vector further comprises a DNA sequence coding for a selectable marker.

In aspects, the selectable marker is an antibiotic resistant selectable marker.

In aspects, the antibiotic resistant selectable marker is aadA.

In aspects, the plastid is a chloroplast, a chromoplast, an amyloplast, a proplastid, a leucoplast or an etioplast.

In aspects, the plastid is a chloroplast.

In aspects, the plastid is a chromoplast.

In aspects, the plastid is an amyloplast.

In aspects, the plastid is a proplastid.

In aspects, the plastid is a leucoplast.

In aspects, the plastid is an etioplast.

In aspects, the plastid is from a monocot or dicot plant.

In aspects, the plastid is from a monocot plant.

In aspects, the plastid is from a dicot plant.

In aspects, the dicot plant is a low-nicotine tobacco plant.

In aspects, the protein of interest is a cytokine.

In aspects, the protein of interest is IL-38.

In aspects, the protein of interest is IL-38b.

In aspects, the protein of interest is IL-37.

In aspects, the protein of interest is IL-37b.

In aspects, the protein of interest is IL-33.

In aspects, the protein of interest is G-CSF.

In aspects, the protein of interest is IL-11.

In aspects, the protein of interest is IL-33.

In aspects, the protein of interest is IL-1Ra.

In aspects, the protein of interest is IL-36Ra.

In aspects, the protein of interest is IL-2.

In aspects, the protein of interest is IL-3.

In aspects, the protein of interest is IL-10.

In aspects, the protein of interest is CSF3.

In aspects, the protein of interest is IL-13.

In aspects, the protein of interest is FGF19.

In aspects, the protein of interest is CSF23.

In aspects, the protein of interest is IL-35.

In aspects, the protein of interest is leukemia inhibitory factor (LIF).

In aspects, the protein of interest is IL-6.

In aspects, the protein of interest is IL-4.

In aspects, the protein of interest is BMP2.

In aspects, the protein of interest is BMP7.

In aspects, the protein of interest is TGF-γ1.

In aspects, the protein of interest is Staphylococcus aureus (Staph aureus) Protein A (Protein A).

In aspects, the protein of interest is encoded by a polynucleotide having the sequence defined by SEQ ID NO. 3, SEQ ID NO. 5, SEQ ID NO 6, or SEQ ID NO 12.

In aspects, the protein of interest is encoded by a polynucleotide having the sequence defined by SEQ ID NO. 3.

In aspects, the protein of interest is encoded by a polynucleotide having the sequence defined by SEQ ID NO. 5.

In aspects, the protein of interest is encoded by a polynucleotide having the sequence defined by SEQ ID NO. 6.

In aspects, the protein of interest is encoded by a polynucleotide having the sequence defined by SEQ ID NO. 12.

In aspects, the protein of interest is at least about 70% identical to a protein encoded by a polynucleotide having the sequence defined by SEQ ID NO. 3, SEQ ID NO. 5, SEQ ID NO. 6 or SEQ ID NO. 12, and wherein the protein of interest substantially retains the biological activity of the polynucleotide having the sequence defined by SEQ ID NO. 3, SEQ ID NO. 5, SEQ ID NO. 6 or SEQ ID NO. 12.

In aspects, the protein of interest is at least about 70% identical to a protein encoded by a polynucleotide having the sequence defined by SEQ ID NO. 3 or SEQ ID NO. 5, and wherein the protein of interest substantially retains the biological activity of the polynucleotide having the sequence defined by SEQ ID NO. 3 or SEQ ID NO. 5.

In aspects, the protein of interest is at least about 70% identical to a protein encoded by a polynucleotide having the sequence defined by SEQ ID NO. 3 and wherein the protein of interest substantially retains the biological activity of the polynucleotide having the sequence defined by SEQ ID NO. 3.

In aspects, the protein of interest is at least about 70% identical to a protein encoded by a polynucleotide having the sequence defined by SEQ ID NO. 5 and wherein the protein of interest substantially retains the biological activity of the polynucleotide having the sequence defined by SEQ ID NO. 5.

In aspects, the protein of interest is at least about 70% identical to a protein encoded by a polynucleotide having the sequence defined by SEQ ID NO. 6 and wherein the protein of interest substantially retains the biological activity of the polynucleotide having the sequence defined by SEQ ID NO. 5.

In aspects, the protein of interest is at least about 70% identical to a protein encoded by a polynucleotide having the sequence defined by SEQ ID NO. 12 and wherein the protein of interest substantially retains the biological activity of the polynucleotide having the sequence defined by SEQ ID NO. 5.

In aspects, the protein of interest is expressed in an amount of about 0.1% to about 60% of total soluble protein (TSP).

In aspects, the protein of interest is present in the plastid in an amount of at least about 0.1% TSP.

In aspects, the protein of interest is present in the plastid in an amount of more than about 0.1% TSP.

In aspects, the protein of interest is present in the plastid in an amount of about 0.1% TSP or more.

In aspects, the protein of interest is present in the plastid in an amount of up to about 60% TSP.

In aspects, the protein of interest is present in the plastid in an amount of about 0.1%, about 0.5%, about 1%, about 5%, about 10%, about 15%, about 20%, about 25%, about 30%, about 35%, about 40%, about 45%, about 50%, about 55%, or about 60% TSP.

In aspects, the protein of interest is present in the plastid in an amount of about 0.1% TSP.

In aspects, the protein of interest is present in the plastid in an amount of about 0.5% TSP.

In aspects, the protein of interest is present in the plastid in an amount of about 1% TSP.

In aspects, the protein of interest is present in the plastid in an amount of about 5% TSP.

In aspects, the protein of interest is present in the plastid in an amount of about 10% TSP.

In aspects, the protein of interest is present in the plastid in an amount of about 15% TSP.

In aspects, the protein of interest is present in the plastid in an amount of about 20% TSP.

In aspects, the protein of interest is present in the plastid in an amount of about 25% TSP.

In aspects, the protein of interest is present in the plastid in an amount of about 30% TSP.

In aspects, the protein of interest is present in the plastid in an amount of about 35% TSP.

In aspects, the protein of interest is present in the plastid in an amount of about 40% TSP.

In aspects, the protein of interest is present in the plastid in an amount of about 45% TSP.

In aspects, the protein of interest is present in the plastid in an amount of about 50% TSP.

In aspects, the protein of interest is present in the plastid in an amount of about 55% TSP.

In aspects, the protein of interest is present in the plastid in an amount of about 60% TSP.

In aspects, the protein of interest is present in an amount of at least about 10% TSP.

In aspects, the protein of interest is present in an amount of about 10% TSP or more.

In aspects, the protein of interest is present in the plastid as a monomer.

In aspects, the protein of interest is present in the plastid as a multimer.

In aspects, the protein of interest is present in the plastid as dimer.

In aspects, the protein of interest is present in the plastid as a trimer.

In aspects, the protein of interest is present in the plastid as a trimer or additional forms of multimers in amount of about 0.1% TSP to about 60% TSP.

In aspects, the protein of interest is present in the plastid as a monomer, a dimer, a trimer or additional forms of multimers in amount of about 0.1% TSP to about 60% TSP.

In aspects, the protein of interest is present in the plastid as a monomer in amount of about 0.1% TSP to about 60% TSP.

In aspects, the protein of interest is present in the plastid as a multimer in amount of about 0.1% TSP to about 60% TSP.

In aspects, the protein of interest is present in the plastid as a dimer in amount of about 0.1% TSP to about 60% TSP.

In aspects, the protein of interest is present in the plastid as a trimer in amount of about 0.1% TSP to about 60% TSP.

In aspects, the protein of interest is present in the plastid as a mixture of a monomer and multimers in amount of about 0.1% TSP to about 60% TSP.

In aspects, the expression cassette comprises a marker to allow the determination of the applicability and functionality thereof by examining the expression of the marker.

In aspects, the marker is a protein.

In aspects, the marker is a recombinant protein.

In aspects, the marker is a fluorescent marker.

In aspects, the marker is a fluorescent protein.

In aspects, the marker is green fluorescent protein (GFP).

In aspects, the marker is a GFP derivative.

In aspects, the marker is blue fluorescent protein.

In aspects, the marker is a blue fluorescent protein derivative.

In aspects, the marker is EBFP.

In aspects, the marker is EBFP2.

In aspects, the marker is Azurite.

In aspects, the marker is mKalama1.

In aspects, the marker is a yellow fluorescent protein.

In aspects, the marker is a yellow fluorescent protein derivative.

In aspects, the marker is Citrine.

In aspects, the marker is Venus.

In aspects, the marker is YPet.

In aspects, the marker is a cyan fluorescent protein.

In aspects, the marker is a cyan fluorescent protein derivative.

In aspects, the marker is ECFP.

In aspects, the marker is Cerulean.

In aspects, the marker is CyPet.

In aspects, the marker is mTurquoise2.

In aspects, the marker is red fluorescent protein.

In aspects, the marker is dsRed.

In aspects, the marker is eqFP611.

In aspects, the marker is Dronpa.

In aspects, the marker is TagRFPs.

In aspects, the marker is KFP.

In aspects, the marker is EosFP/IrisFP.

In aspects, the marker is Dendra.

In another aspect, there is provided a transformation vector for stably transforming a plastid, comprising, an expression cassette, comprising, as operably-linked components, a promoter comprising psbA operative in the plastid, a heterologous polynucleotide encoding IL-38, and, flanking each side of the expression cassette, a first DNA flanking sequence comprising trnl and a second flanking DNA sequence comprising trnA which allow for stable integration of the heterologous polynucleotide sequence encoding IL-38 into the plastid genome.

In aspects, the expression cassette further comprises a spacer region between psbN and psbH genes.

In aspects, the expression cassette further comprises a 5′UTR comprising 77G10.

In aspects, the expression cassette further comprises a 3′ UTR comprising psbC.

In aspects, the expression cassette further comprises a selectable marker comprising aadA.

In another aspect, there is provided a transformation vector for stably transforming a plastid, comprising, an expression cassette, comprising, as operably-linked components, a promoter comprising psbA operative in the plastid, a heterologous polynucleotide encoding protein A of Staph aureus, and, flanking each side of the expression cassette, a first DNA flanking sequence comprising trnl and a second flanking DNA sequence comprising trnA which allow for stable integration of the heterologous polynucleotide sequence encoding protein A of Staph aureus into the plastid genome.

In aspects, the expression cassette further comprises a spacer region between psbN and psbH genes.

In aspects, the expression cassette further comprises a 5′UTR comprising 77G10.

In aspects, the expression cassette further comprises a 3′ UTR comprising psbC.

In aspects, the expression cassette further comprises a selectable marker comprising aadA.

In another aspect, there is provided a transformation vector for stably transforming a plastid, comprising, an expression cassette, comprising, as operably-linked components, a promoter comprising psbA operative in the plastid, a heterologous polynucleotide encoding IL-37b, and, flanking each side of the expression cassette, a first DNA flanking sequence comprising trnl and a second flanking DNA sequence comprising tmnA which allow for stable integration of the heterologous polynucleotide sequence encoding IL-37b into the plastid genome.

In aspects, the expression cassette further comprises a spacer region between psbN and psbH genes.

In aspects, the expression cassette further comprises a 5′UTR comprising psbA.

In aspects, the expression cassette further comprises a 3′ UTR comprising psbC.

In aspects, the expression cassette further comprises a selectable marker comprising aadA.

In another aspect, there is provided a transformation vector for stably transforming a plastid, comprising, an expression cassette, comprising, as operably-linked components, a promoter comprising psbA operative in the plastid, a heterologous polynucleotide encoding IL-33, and, flanking each side of the expression cassette, a first DNA flanking sequence comprising trnl and a second flanking DNA sequence comprising trnA which allow for stable integration of the heterologous polynucleotide sequence encoding IL-33 into the plastid genome.

In aspects, the expression cassette further comprises a spacer region between psbN and psbH genes.

In aspects, the expression cassette further comprises a 5′UTR comprising psbA.

In aspects, the expression cassette further comprises a 3′ UTR comprising psbC.

In aspects, the expression cassette further comprises a selectable marker comprising aadA.

In another aspect, there is provided a transformation vector for stably transforming a plastid, comprising, an expression cassette, comprising, as operably-linked components, a promoter comprising psbA operative in the plastid, a heterologous polynucleotide encoding G-CSF, and, flanking each side of the expression cassette, a first DNA flanking sequence comprising trnl and a second flanking DNA sequence comprising trnA which allow for stable integration of the heterologous polynucleotide sequence encoding G-CSF into the plastid genome.

In aspects, the expression cassette further comprises a spacer region between psbN and psbH genes.

In aspects, the expression cassette further comprises a 5′UTR comprising psbA.

In aspects, the expression cassette further comprises a 3′ UTR comprising psbC.

In aspects, the expression cassette further comprises a selectable marker comprising aadA.

In another aspect, there is provided a plant plastid stably transformed with the transformation vector described herein.

In another aspect, there is provided a plant cell stably transformed with the transformation vector described herein.

In another aspect, there is provided a plant stably transformed with the transformation vector described herein.

In aspects, the plant further comprises mature leaves transformed with the vector described herein.

In aspects, the plant further comprises young leaves transformed with the vector described herein.

In aspects, the plant further comprises old leaves transformed with the vector described herein.

In another aspect, there is provided a progeny of the plant described herein.

In another aspect, there is provided a seed of the plant described herein.

In another aspect, there is provided a method for producing a protein of interest comprising: integrating the transformation vector described herein into a plastid genome of a plant cell; and growing the plant cell to thereby express the protein of interest.

In aspects, the plastid genome is from a tobacco plant.

In aspects, the plastid genome is from a low-alkaloid tobacco plant.

In aspects, the low-alkaloid tobacco plant is low-alkaloid cultivar 81V9.

In aspects, the method further comprises recovering the protein of interest.

In aspects, the recovering comprises isolating and purifying the protein of interest.

In aspects, the purifying comprises using an immobilized metal-affinity chromatography (IMAC) procedure.

In aspects, the protein of interest is competent to modulate an immune response ex vivo or in vitro.

In aspects, the protein of interest is competent to modulate an immune response ex vivo.

In aspects, the protein of interest is competent to modulate an immune response in vitro.

In aspects, the protein of interest dose-dependently modulates the immune response.

In aspects, the immune response comprises modulation of peripheral blood mononuclear cells cytokine secretion in response to inflammatory mediator stimulation.

In aspects, the inflammatory mediator comprises LPS or PHA.

In aspects, the inflammatory mediator comprises LPS.

In aspects, the inflammatory mediator comprises PHA.

In aspects, the immunogenic response comprises modulation of tissue cell cytokine secretion in response to viral stimulation.

In aspects, the viral stimulation is SARS-COV-2 stimulation.

In aspects, the tissue cell is a lung cell.

In another aspect, there is provided a protein of interest for use in the modulation of an immune response, wherein the protein of interest is obtainable by any method for producing the protein of interest described herein.

In another aspect, there is provided a use of the transformation vector described herein for stably transforming a plant cell.

In another aspect, there is provided a use of the transformation vector described herein for stably transforming a plant.

In another aspect, there is provided a use of the transformation vector described herein for producing the protein of interest that can modulate an immune response.

In aspects, the immune response comprises cytokine secretion from peripheral blood mononuclear cells.

In aspects, the immune response comprises cytokine secretion from tissue cells in response to SARS-COV-2 stimulation.

In another aspect, there is provided a method of treating an inflammatory disorder comprising administering the protein of interest produced from the transformation vector described herein to a patient in need thereof.

In another aspect, there is provided a use of the protein of interest produced from the transformation vector described herein for treating an inflammatory disorder in a patient in need thereof.

In another aspect, there is provided a composition comprising at least one excipient and the protein of interest produced from the transformation vector described herein.

In another aspect, there is provided a use of the composition described herein for treating an inflammatory disorder in a patient in need thereof.

In another aspect, there is provided a protein of interest produced from the transformation vector described herein.

In another aspect, there is provided a protein of interest produced by the method described herein.

In another aspect, there is provided a use of the protein of interest described herein for treating an inflammatory disorder in patient in need thereof.

The novel and inventive features of the present invention will become apparent to those of skill in the art upon examination of the following detailed description of the invention. It should be understood, however, that the detailed description of the invention and the specific examples presented, while indicating certain aspects of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The present invention will be further understood from the following description with reference to the Figures, in which:

FIG. 1 shows schematic representation of chloroplast expression cassettes pSGB-16S-GOI #, pSGB-V12-GOI # and pSGB-GT-GOI # in accordance with the present invention.

FIG. 2 shows a photograph illustrating different expression and accumulation levels of recombinant green fluorescent protein (rGFP) observed in leaves of plastome-engineered tobacco plants. Left panel: leaves from the wild-type (WT) tobacco, as well as from three different bioreactor lines (B1, B2. B3) appear healthy green under normal light; Right panel: under the UV light different levels of rGFP fluorescence are visible in B1, B2 and B3 leaves, contrasting the strong maroon chlorophyll autofluorescence of the WT leaf.

FIG. 3 shows SDS-PAGE and staining of fresh leaf tissue extracts from wild-type (WT) and the transplastomic bioreactor line (B3-rGFP). Extract equivalents of 1 mg of fresh leaf tissue collected from young (Y), mature (M), mature-old (MO) and senescing(S) leaves (see right panel) were separated by electrophoresis and stained to reveal the profile of total soluble protein (TSP) content. Molecular Weight (MW) ladder is in kilodaltons (kDa). The major band of ˜50 kDa present in samples from both WT and B3 genotypes is the RuBisCO large subunit, which usually constitutes-40%-50% of TSP. The major band of 32 kDa found in the B3 bioreactor line represents the recombinant GFP produced in chloroplasts, clearly more abundant than the RuBisCO in M, MO and S leaves.

FIG. 4 shows (A) SDS-PAGE separation and staining of the in planta-produced, purified rGFP and detection of the rGFP on western blots with anti-HIS-tag (α-HIS), anti-c-myc tag (α-c-myc) or anti-GFP (α-GFP) antibodies. Molecular Weight (MW) ladder is in kilodaltons (kDa); and (B) Images of the purified rGFP protein under daylight (left panel) and UV light (right panel). Strong green fluorescence is visible under UV light.

FIG. 5 shows SDS-PAGE-Western blot analysis of the extracts from the original clones regenerated (First Regeneration, IR) on the selective medium after the transformation with pSGB-16S-GOI #constructs. Each lane contains extract of 2 mg of plantlet leaf tissue. Image exposure time is indicated in seconds in the low right corner of the blot. Pairs of extracts of two independent clones (C1, C2) for each GOI genotype are shown; lane 14, WT negative control, lane 15 contains 25 ng of CBD-c-myc positive control protein. * Two sub-clones of the single original clone.

FIG. 6 shows SDS-PAGE+stain analysis of the purified samples from clones expressing GOI #1 (rhIL-38), GOI #3 (rhIL-37b), GOI #4 (rhIL-33), GOI #7 (rhIL-2) and GOI #10 (rhG-CSF) (lanes 2-6, respectively). Monomeric forms are indicated with a single black triangle, dimers are indicated with double black triangle. Each lane contains protein extract eluted from 5 μL of resin from the c-myc-tagged protein mild purification kit ver.2 (MBL International), using ˜500 mg leaf tissue harvested from IR clones; standard curve (lanes 7, 8, 9, 10) contains 100, 200, 400, 800 ng of the Bovine Serum Albumin (BSA) protein, respectively.

FIG. 7 shows Left panel: SDS-PAGE-Western blot analysis of the extracts of leaf samples from rooted clones expressing GOI #1, grown in greenhouse (left panel). Each lane contains extract of 1 mg leaf tissue. The expected protein size is 18.3 kDa. Triplets of young (Y), mature (M) and senescing(S) leaf samples from 3 different clones; WT—wild type negative control; standard curve contains 6.25, 12.5, 25, 50 ng of CBD-c-myc control protein. On the right panel, a dilution series (from 1 mg/lane to 0.1 mg/lane) of the extract of the GOI #1-(best) expressing senescing leaf tissue analyzed by SDS-PAGE-Western blot. Estimated recombinant protein (monomer) yield is-500 ng/mg leaf tissue.

FIG. 8 shows SDS-PAGE-Western blot analysis of the extracts of leaf samples from rooted clones expressing GOI #3, grown in greenhouse (left panel). Each lane contains extract of 1 mg leaf tissue. The expected protein size is 20.3 kDa. Triplets of young (Y), mature (M) and senescing(S) leaf samples from 3 different clones; WT-wild type negative control; standard curve contains 6.25, 12.5, 25, 50 ng of CBD-c-myc control protein. On the right panel, a dilution series (from 1 mg/lane to 0.1 mg/lane) of the extract of the senescing leaf tissue analyzed by SDS-PAGE-Western blot. Estimated recombinant protein (monomer) yield is ˜750 ng/mg leaf tissue.

FIG. 9 shows SDS-PAGE-Western blot analysis of the extracts of leaf samples from rooted clones expressing GOI #4, grown in greenhouse. Each lane contains extract of 1 mg leaf tissue. The expected protein size is 27.3 kDa. Triplets of young (Y), mature (M) and senescing(S) leaf samples from 3 different clones: WT-wild type negative control; standard curve contains 6.25, 12.5, 25, 50 ng of CBD-c-myc control protein.

FIG. 10 shows SDS-PAGE-Western blot analysis of the extracts of leaf samples from rooted clones expressing GOI #7, grown in greenhouse. Each lane contains extract of 1 mg leaf tissue. The expected protein size is 16.2 kDa. Triplets of young (Y), mature (M) and senescing(S) leaf samples from 3 different clones; WT-wild type negative control; standard curve contains 6.25, 12.5, 25, 50 ng of CBD-c-myc control protein.

FIG. 11 shows SDS-PAGE-Western blot analysis of the extracts of leaf samples from rooted clones expressing GOI #10, grown in greenhouse. Each lane contains extract of 1 mg leaf tissue. The expected protein size is 20.1 kDa. Triplets of young (Y), mature (M) and senescing(S) leaf samples from 3 different clones; WT-wild type negative control; standard curve contains 6.25, 12.5, 25, 50 ng of CBD-c-myc control protein.

FIG. 12 shows SDS-PAGE+staining of samples of the eluted proteins IMAC-purified from crude extracts of leaf tissue from B3-GOI1-7H (right panel) and B2-GOI3-7H (left panel) bioreactor lines grown in greenhouse. Molecular Weight (MW) ladder is in kilodaltons (kDa). Single black triangle arrows depict the monomers of the recombinant human cytokines of the predicted molecular sizes (18.3 kDa and 20.3 kDa for rhIL-38 and rhIL-37, respectively), double arrows depict the dimers. Higher molecular weight multimeric structures are also detectable. From the left: Lane 1-molecular size marker in kiloDaltons (kDa), lanes 2-5-different amounts of the purified Plantakines, lane 6-rGFP (500 ng), lanes 7-10-different amounts of Bovine Serum Albumin (BSA).

FIG. 13 shows SDS-PAGE+western blots with protein-specific antibodies of samples of the in planta-produced and purified plantakines rhIL-37b and rhIL-38 from bioreactor lines grown in greenhouse. On the left panel, Lane 1: plant-produced rhIL-37b; Lane 2: recombinant bacterial-produced IL-37b (150 ng, R&D Biosystems, Cat. #7585-IL-025/CF; antibody: MyBiosource, Cat. #MBS668098); on the right panel, Lane 1: plant-produced rhIL-38; Lane 2: recombinant bacterial-produced IL-38 (150 ng, MyBiosource, Cat. #MBS635478; antibody: R&D Biosystems, Cat. #MAB7774). Molecular Weight (MW) ladder is in kilodaltons. Anti-IL37b antibody detected rhIL-37b in two major forms: monomers (20.3 kDa) and dimers, present in comparable amounts. Anti-IL38 antibody detected rhIL-38 in several major forms: monomers (18.3 kDa), dimers, trimers, tetramers are detectable in comparable amounts.

FIG. 14 shows SDS-PAGE and staining of samples of IMAC-purified and eluted fractions from crude extracts of fresh leaf tissue from B3-StpA transplastomic genotypes grown in greenhouse. Molecular Weight (MW) ladder is in kilodaltons (kDa). Two extraction buffers were tested for purification: Sodium Acetate buffer (NaAc), pH=4.9 and Phosphate buffer (Phos), pH=7.4. Single black triangle arrow depicts the band of the recombinant protein A of the predicted molecular size of 35.4 kDa.

FIG. 15 shows expression and Purification of the Plantakines IL-37b and IL-38, a: SDS-PAGE and Western blots of samples from crude leaf tissue extracts of the primary transplastomic clones generated for expression of plantakines IL-37b and IL-38 tagged with a HIS-tag at the C-terminus. Numbers 1-5 for each IL-37b and IL-38 represent extracts (˜100 μg fresh leaf tissue) from different clones; clones 1, 2 and 5 for IL-38 show no expression. VGFP (EGEH (Ramirez-Alanis et al., 2018)) is a HIS-tagged GFP variant used as quantifiable control protein; lanes 1, 2 and 3 represent 12.5, 25 and 50 ng, respectively. All blots probed with the same anti-His tag antibody, b: Left panel: Schematic representation of a greenhouse-grown bioreactor plant assessed to determine the spatial expression patterns of the cytokines by sampling young (Y), mature (M) and old (O) leaves; Right panel: Three clones (C1, C2, C3) for each bioreactor line expressing either IL-37b or IL-38 were sampled (˜1 mg fresh leaf tissue in lane) and assessed with Western blots. Wild-type (WT) tobacco extracts were used as negative controls. VGFP was used as quantifiable control protein; lanes 1, 2 and 3 represent 12.5, 25 and 50 ng, respectively, c: Lanes 1 and 2 both contain ˜1 μg of the purified plantakine IL-37b, SDS-PAGE & stained (lane 1) or Western-blotted and probed with anti-IL-37 antibody (lane 2) along with 500 ng of bacteria-produced human recombinant IL-37b as a control (lane 3), d: Lanes 1 and 2 both contain ˜1 μg of the purified plantakine IL-38, SDS-PAGE & stained (lane 1) or Western-blotted and probed with anti-IL-38 antibody (lane 2) along with 500 ng of bacteria-produced human recombinant IL-38 as a control (lane 3). Molecular weight marker (MW) ladder is in kiloDaltons. Single black triangle arrows depict the monomers of the plantakines of the predicted molecular sizes (20.3 kDa and 18.3 kDa for IL-37b and IL-38, respectively), double arrows depict the dimers. Higher molecular weight multimeric structures are also detectable.

FIG. 16 shows a graph illustrating differences in levels of inflammatory cytokines secreted from PBMCs in response to stimulation with either LPS or PHA and effects of IA dosage. Statistically significant differences are indicated for mean concentration values (+/−SEM) obtained for each marker under LPS or PHA stimulation, as well as differences between the low and high doses within the applied IA. Baseline secretion from PBMCs for each of the markers was significantly different (p<0.001) than the mean concentration secreted in response to either IA. Legend: n/s-no significant difference; *-significant difference, p<0.05; ***-significant difference, p<0.001.

FIG. 17 shows occupancy of the Zinc ion in the Zinc knuckle motif with respect to the pH values. Data is derived from MultiConformer Continuum Electrostatics (MCCE) Monte-Carlo Simulation where the y-axis corresponds to occupancy from 0 to 1 and the x-axis corresponds to pH values. The H80R mutation causes a dramatic change in the electrostatic environment resulting in a large change in the occupancy.

FIG. 18 shows an alignment of the NSP10-derived sequence employed to inhibit the replication of Murine Hepatitis Virus (MHV) with full length NSP10 sequences from SARS-COV, SARS-COV-2, and MERS-COV. * indicates a Proline to Valine substitution in MHV and MERS-CoV compared with SARS-COV and SARS-COV-2; # indicates residue His80 in SARS-COV and SARS-COV-2.

FIG. 19 shows a graph illustrating that SARS-COV-2 NSP10-derived sequences cause an increase in IL-6 secretion by human lung cancer cells. The human NSCLC cell line NCI-H1792 was incubated in the presence of NSP10-derived peptides P1 or P2, harbouring an N-terminal HIV-TAT sequence, or the TAT-only peptide for 24 h. The secretion of IL-6 was measured by ELISA and normalized to the control (PBS). Shown are the average results of 3 independent experiments. *** P<0.001.

FIG. 20 shows a graph illustrating that IL-37b and IL-38 attenuate the Peptide 1-induced stimulation of IL-6 secretion by human cells. IL-6 secretion by the human NSCLC cell line NCI-H1792 into the conditioned media was measured by ELISA. The cells were pre-incubated for 3 h in the presence of plant-produced recombinant IL-38, IL-37b (1.0 ng/ml), or a combination of IL-38+IL-37b (0.5 ng/ml each), as indicated. PBS (1×) and a His-tag containing plant-produced GFP were used as controls. IL-6 secretion was stimulated by the addition of NSP10-derived Peptide 1 (10 mM) for 24 h. Shown are the average results of 4 independent experiments. Different letters above the columns indicate significant differences.

FIG. 21 shows a graph illustrating that IL-37b and IL-38 attenuate the Peptide (P) 1-induced stimulation of IL-6 secretion by human cells in a dose-dependent manner. IL-6 secretion by the human metastatic breast cancer cell line MDA-MB-231 into the conditioned media was measured by ELISA. The cells were pre-incubated for 3 h in the presence of plant-produced recombinant IL-38 or IL-37b at the indicated concentrations (ng/ml) prior to stimulation with peptide P1 (10 mM). PBS (1×) was added to the control. Shown are the average results of 3 independent experiments. ** P<0.01; * P<0.05, compared with the control.

FIG. 22 shows the structure of nsp10 of SARS-COV (blue) SARS-COV-2 (magenta) and MERS-COV (yellow). The mutation from Proline to Valine is shown in red circle.

FIG. 23 shows atomic fluctuations as measured by the DynaMut server. Left is MERS, right is SARS-COV-2. Red indicate instability while blue indicates stability. Red circle indicates Zinc knuckle motif.

FIG. 24 shows modulations of secretion of select inflammatory markers (IFNγ, GM-CSF, IL-17 and IL-22) by stimulated PBMCs after treatments with different doses of the plant-produced recombinant IL-37b and IL-38. The effects of the treatments were calculated as percentages (Level Modulations, the “Y” axis) of secretion modulation with its probability value, derived from comparison with the positive control levels (0%, the “X” axis) at the corresponding IAs concentrations for each inflammatory marker monitored.

FIGS. 25a and 25b shows modulations of inflammatory markers secretion by stimulated PBMCs after treatments with different doses of the plant-produced recombinant IL-37b and IL-38. The effects of the treatments were calculated as percentages (Level Modulations, the “Y” axis) of secretion modulation with its probability value in comparison with the positive controls (0%, the “X” axis) at the corresponding IAs concentrations for each inflammatory marker monitored.

FIG. 26 shows antibody mAb53 binding and elution from the chromatography resin manufactured with plant-produced protein A ligand. SDS-PAGE results of the initial antibody solution (input) loaded on the column, the flow-through fraction, and the four consecutive elutions of the bound antibody mAb53, showing the heavy and the light chains of the molecule.

### DETAILED DESCRIPTION

### Definitions

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

“Spacer region” or “Spacer” is understood in the art to be the region between two genes. The chloroplast genome of plants contains spacer regions which are non-coding, untranslated and non-conserved sequences between highly conserved nucleotide gene sequences, that often undergo processing by endogenous nucleases. It is well understood in the art that the sequences flanking functional genes are well-known to be called “spacer regions” or “spacers”. The special features of the spacer region are described in U.S. Pat. No. 7,129,391, hereby incorporated by reference. It was well-known that there are at least sixty transcriptionally-active spacer regions within the higher plant chloroplast genomes (Sugita, M., Sugiura. M., Regulation of Gene Expression in Chloroplasts of Higher Plants, Plant Mol. Biol, 32:315-326, 1996, hereby incorporated by reference). Specifically, Sugita et al. reported sixty transcriptionally-active spacer regions referred to as transcription units, as can be seen in Table Il of the article.

“Selectable marker” provides a means of selecting the desired plant cells, vectors for plastid transformation typically contain a construct which provides for expression of a selectable marker gene. “Marker genes” are plant-expressible DNA sequences which express a polypeptide which resists a natural inhibition by, attenuates, or inactivates a selective substance, i.e., antibiotic or herbicide. Numerous additional promoter regions may also be used to drive expression of the selectable marker gene, including various plastid promoters and bacterial promoters which have been shown to function in plant plastids.

Alternatively, a selectable marker gene may provide some other visibly reactive response, i.e., may cause a distinctive appearance or growth pattern relative to plants or plant cells not expressing the selectable marker gene in the presence of some substance, either as applied directly to the plant or plant cells or as present in the plant or plant cell growth media.

In either case, the plants or plant cells containing such selectable marker genes will have a distinctive phenotype for purposes of identification, i.e., they will be distinguishable from non-transformed cells. The characteristic phenotype allows the identification of cells, cell groups, tissues, organs, plant parts or whole plants containing the construct. Detection of the marker phenotype makes possible the selection of cells having a second gene to which the marker gene has been linked. The use of such a marker for identification of plant cells containing a plastid construct has been described in the literature.

When referring to the relative age of the plants, plant parts and leaves, well followed principles in the art should be applied. Young, mature and old plants are considered in the cycle of plant life. Young reproductive plants exhibit more new growth than death of old parts. Mature plants exhibit a balance between growth and death of parts. These plants usually have the greatest yearly seed production and biomass increase (increase in weight). In other words, they are at their peak. In old plants, the death of parts prevails over the production of new parts. Reproductive activity is diminished.

Inverted Repeat Regions (known as IR) are regions of homology, which are present in most plastid genomes explored hitherto, two copies of the transgene are expected per transformed plastome in the case when the expression cassette is integrated into IR. In the case when integration of the expression cassette is outside the inverted repeat regions, then one copy of the transgene is expected per transformed plastome.

An “immune response” is referred to as the physiological responses stemming from the activation of the immune system by antigens and manifested through changes in levels of secreted inflammation markers. In the present invention, the immune response of peripheral blood mononuclear cells (PBMCs), or of tissue cells, such as lung cells, may be modulated when those cells are treated with the proteins of interest as described herein.

An “autoantigen” is any substance normally found within an animal that, in an abnormal situation, such as an autoimmune disease, is no longer recognized as part of the animal itself by the immune system of that animal and is therefore attacked by the immune system as though it were a foreign substance.

An “allergen” is an antigen that induces an allergic reaction of a host.

A “plant” refers to any plant particularly to seed plants.

“Plant cell” refers to the structural and physiological unit of the plant, comprising a protoplast and a cell wall. The plant cell may be in the form of an isolated single cell or a cultured cell, or as a part of higher organized unit such as, for example, a plant tissue, or a plant organ.

“Plant material” refers to leaves, stems, roots, seeds, flowers or flower parts, fruits, pollen, pollen tubes, ovules, embryo sacs, egg cells, zygotes, embryos, seeds, cuttings, cell or tissue cultures, or any other part or product of a plant.

“Plant matter” refers to any part of a plant at any stage of development, preferably such parts that can be administered orally. Plant matter includes edible parts of a plant, such as leaves, seeds, fruits, tubers, or other plant parts that can be ingested raw or unprocessed. Plant matter also includes isolated fractions of the plants, such as subcellular organelles, e.g. plastids or vacuoles.

“Expression” refers to the transcription and translation of an endogenous gene or a transgene in plants. In the case of antisense constructs, for example, expression may refer to the transcription of the antisense DNA only.

“Expression cassette” as used herein means a DNA sequence capable of directing expression of a particular nucleotide sequence in an appropriate host cell, comprising a promoter operably linked to the nucleotide sequence of interest which is optionally operably linked to 3′ sequences, such as 3′ regulatory sequences or transcription termination signals. It also typically comprises sequences required for proper translation of the nucleotide sequence. The coding region usually codes for a protein of interest but may also code for a functional RNA of interest, for example antisense RNA, or double-stranded RNA, or a non-translated RNA that, in the sense or antisense direction, inhibits expression of a particular gene, e.g., antisense RNA. The expression cassette comprising the nucleotide sequence of interest may be chimeric, meaning that the nucleotide sequence is comprised of more than one DNA sequences of distinct origin, which are fused together by recombinant DNA techniques resulting in a nucleotide sequence which does not occur naturally, and which particularly does not occur in the plant to be transformed. The expression cassette may also be one which is naturally occurring but has been obtained in a recombinant form useful for heterologous expression.

Typically, however, the expression cassette is heterologous with respect to the host, i.e., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter which initiates transcription only when the host cell is exposed to some particular external stimulus. In the case of a multicellular organism, such as a plant, the promoter can also be specific to a particular tissue or organ or stage of development. A plastid expression cassette is usually inserted into the plastid genome of a plant and is capable of directing the expression of a particular nucleotide sequence from the plastid genome of said plant. In the case of a plastid expression cassette, for expression of the nucleotide sequence from a plastid genome, additional elements, i.e. ribosome binding sites, 5′ and 3′ UTR stem-loop structures that impede plastid RNA degradation may be required.

A “transformation vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, and DNA plasmids.

“Gene” refers to a coding sequence and associated regulatory sequences wherein the coding sequence is transcribed into RNA such as mRNA, rRNA, IRNA, snRNA, sense RNA or antisense RNA. Examples of regulatory sequences are promoter sequences, 5′ and 3′ untranslated sequences (UTRs) and transcription termination sequences. Further elements that may be present are, for example, introns.

“Heterologous” as used herein means of different natural or of synthetic origin. For example, if a host cell is transformed with a nucleic acid sequence that does not occur in the untransformed host cell, that nucleic acid sequence is said to be heterologous with respect to the host cell. The transforming nucleic acid may comprise a heterologous promoter, heterologous coding sequence, or heterologous termination sequence. Alternatively, the transforming nucleic acid may be completely heterologous or may comprise any possible combination of heterologous and endogenous nucleic acid sequences. Similarly, heterologous refers to a nucleotide sequence derived from and inserted into the same natural, original cell type, but which is present in a non-natural state, e.g. a different copy number, or under the control of different regulatory elements.

“Regulatory elements” or “regulatory sequence” refer to sequences involved in conferring the expression of a nucleotide sequence. A regulatory DNA sequence is said to be “operably linked to” or “associated with” a DNA sequence that codes for an RNA or a protein if the two sequences are situated such that the regulatory DNA sequence affects expression of the coding DNA sequence. For example, a promoter is operably linked to a coding sequence if the promoter affects the transcription or expression of the coding sequence.

A “promoter” refers to a DNA sequence that initiates transcription of an associated DNA sequence. The promoter region may also include elements that act as regulators of gene expression such as activators, enhancers, and/or repressors.

“Homoplastomic” or “homoplastomy” refers to a plant, plant tissue or plant cell stage wherein all of the plastids are uniformly transformed and, hence, genetically identical. “Heteroplastomic” or “heteroplastomy” refers to a plant, plant tissue or plant cell stage wherein the plastids are not genetically uniform and present as a mixed population of transformed and untransformed, wild type plastids. In different tissues or stages of development, the plastids may take different forms, e.g., chloroplasts, proplastids, etioplasts, amyloplasts, chromoplasts, and so forth.

“Recombinant DNA technology” refers to procedures used to join together DNA sequences as described, for example, in Sambrook et al., 1989, Cold Spring Harbor, NY: Cold Spring Harbor Laboratory Press.

“Transformation” refers to introduction of a nucleic acid into a cell. In particular, the stable integration of a DNA molecule into the genome of an organism of interest.

The term “transformed” or “transformation” as used herein refers to a process by which exogenous nucleic acid is transferred or introduced into the host cell. A “transformed” cell is one which has been transformed with exogenous nucleic acid. The cell includes the primary subject cell and its progeny.

“Encoding” or “coding for” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of the mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Isolated” refers to a molecule that has been purified from its source or has been prepared by recombinant or synthetic methods and purified. Purified proteins are substantially free of other cellular materials.

By the term “modulating.” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, typically, a human.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the term “protein” refers to a compound comprised of amino acid residues covalently linked by peptide bonds. A protein must contain at least two amino acids, and no limitation is placed on the maximum number of amino acids that can comprise a protein's sequence.

A substantially identical sequence may comprise one or more conservative amino acid mutations. It is known in the art that one or more conservative amino acid mutations to a reference sequence may yield a mutant protein with no substantial change in physiological, chemical, or functional properties compared to the reference sequence; in such a case, the reference and mutant sequences would be considered “substantially identical” proteins. Conservative amino acid mutation may include addition, deletion, or substitution of an amino acid; a conservative amino acid substitution is defined herein as the substitution of an amino acid residue for another amino acid residue with similar chemical properties (e.g. size, charge, or polarity).

Thus, “biologically active” or “biological activity” when used in conjunction with the proteins described herein refers to protein that exhibits or shares an effector function of the native protein. For example, the proteins described herein have the biological activity of modulating immune responses ex vivo and in vitro. “Biologically active” or “biological activity” when used in conjunction with variant sequences means that the variant sequences exhibit or share an effector function of the parent sequence. The biological activity of the variant sequence may be increased, decreased, or at the same level as compared with the parent sequence.

As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable.

The term “subject” or “patient” may be used interchangeably, and refers to any member of the animal kingdom, typically a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

When introducing elements disclosed herein, the articles “a”, “an”, “the”, and “said” are intended to mean that there may be one or more of the elements.

The term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. It will be understood that any embodiments described as “comprising” certain components may also “consist of” or “consist essentially of,” these components, wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effects described herein. For example, a composition defined using the phrase “consisting essentially of” encompasses any known acceptable additive, excipient, diluent, carrier, and the like, suitable for the composition described herein. Typically, a composition consisting essentially of a set of components will comprise less than 5% by weight, typically less than 3% by weight, more typically less than 1% by weight of non-specified components.

It will be understood that any component defined herein as being included may be explicitly excluded from the claimed invention by way of proviso or negative limitation, such as any specific compounds or method steps, whether implicitly or explicitly defined herein.

In addition, all ranges given herein include the end of the ranges and also any intermediate range points, whether explicitly stated or not.

Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The abbreviation, “e.g.” is derived from the Latin exempli gratia, and is used herein to indicate a non-limiting example. Thus, the abbreviation “e.g.” is synonymous with the term “for example.” The word “or” is intended to include “and” unless the context clearly indicates otherwise.

The phrase “at least one of” is understood to be one or more. The phrase “at least one of . . . and . . . ” is understood to mean at least one of the elements listed or a combination thereof, if not explicitly listed. For example, “at least one of A, B, and C” is understood to mean A alone or B alone or C alone or a combination of A and B or a combination of A and C or a combination of B and C or a combination of A, B, and C.

It is to be understood that the terminology used herein is for the purpose of describing particular aspects only, and is not intended to be limiting. Many patent applications, patents, and publications are referred to herein to assist in understanding the aspects described. Each of these references are incorporated herein by reference in their entirety.

Chloroplasts are prokaryotic compartments inside eukaryotic cells. Since chloroplasts have similar transcriptional and translational machinery to E. coli., and the plant cell can contain 10's of 1000's of copies of the circular plastid genome, it may be possible to express prokaryotic and/or eukaryotic genes at very high levels in plant chloroplasts compared to that in the nucleus. Moreover, the chloroplast is capable of allowing for the processing of eukaryotic proteins, including folding and formation of disulfide bridges since the necessary machinery is found within the chloroplast. These include, for example, chaperonin proteins, chloroplast thioredoxin systems and chloroplast protein disulfide isomerases. This can, for example, limit the requirement for further in vitro processing of the plastid-derived proteins described herein. An ideal expression system would be one that produces a maximum amount of safe, biologically active material at a minimum cost.

The use of modified mammalian cells, for example, with recombinant DNA techniques has the advantage of resulting in products, which are closely related to those of natural origin. However, culturing these cells uses a lot of resources and can only be carried out on limited scale. The use of microorganisms such as bacteria, for example, permits manufacture on a larger scale, but introduces the disadvantage of producing products, which differ appreciably from the products of natural origin (e.g., lack of glycosylation of proteins in bacterial systems).

While production of proteins in plants may overcome the obstacles mentioned above, a major limitation in producing proteins in plants is their low level of foreign protein expression. For example, each of hepatitis B surface antigen, Norwalk virus capsid protein and human epidermal growth factor have been shown to be expressed in plants, such as tobacco or potatoes, however, their yield as percentage of total soluble protein (TSP) has been low for each of them at 0.01% TSP; 0.001% TSP; and 0.02% TSP, respectively.

The methods described herein provide for increased levels of expression of biologically active recombinant proteins in plants in a safe and cost-efficient manner, making the plastid, and in particular, the chloroplast, an ideal protein expression system.

### Proteins Expressed by the Method

The present invention typically relates to the production of proteins of interest. The protein is not particularly limited and may, for example, be a therapeutic protein. The protein may be suitable for administration to a human subject; optionally wherein the administration occurs after purification. The protein may be capable of modulating the immune response. For example, the protein may be a cytokine. Preferably, the cytokine is a human cytokine. The protein of interest can be used for downstream applications, such as the modulation of inflammatory responses, as described below.

Advantageously, proteins produced using the methods of the invention may be more stable and/or more likely to form multimers than corresponding proteins produced using bacteria. For example, dimers (and higher molecular weight multimers) may be stable and detectable in SDS-PAGE analyses gels even after harsh denaturing conditions of the sample preparation.

The protein of interest, once produced and purified, can be used for downstream applications, such as, for example, modulation of inflammatory responses, as described below. The protein of interest can be, for example, a blood protein (e.g. clotting factors VIII and IX, complement factors and complements, hemoglobins or other blood proteins, serum albumin, and the like), a hormone (e.g. insulin, growth hormone, thyroid hormone, follicle-stimulating hormone (FSH), catecholamines gonadotrophins, pregnant mare serum gonadotropin (PMSG), trophic hormones, prolactin, oxytocin, dopamine, bovine somatotropin, leptins and the like), a growth factor (e.g. epidermal growth factor (EGF), platelet-derived growth factor (PDGF), nerve growth factor (NGF), insulin growth factor (IGF), fibroblast growth factor (FGF), bone morphogenetic proteins (BMP) and the like), a cytokine (e.g. interleukins (ILs), colony-stimulating factor (CSF), granulocyte colony-stimulating factor (G-CSF), granulocyte macrophage colony-stimulating factor (GM-CSF), erythropoietin (EPO), tumour necrosis factor (TNF), transforming growth factor (TGF)-including TGFα and TGFβ, interferons (IFNs) and the like), an enzyme (e.g. tissue plasminogen activator, streptokinase, cholesterol biosynthetic or degradative, steroidogenic enzymes, kinases, phosphodiesterases, methylases, de-methylases, dehydrogenases, cellulases, proteases, lipases, phospholipases, aromatases, cytochromes, adenylate or guanylate cyclases, neuraminidases and the like), a receptor (e.g. steroid protein, peptide, lipid or prostaglandin, and the like), a binding protein (e.g. steroid binding proteins, growth hormone or growth factor binding proteins, and the like), an immune system protein (e.g. antibodies, antibody fragments, chimeric antibodies, variable regions, or the like, or major histocompatibility complex (MHC) genes), a translation factor, an antibody, an allergen, an oncoprotein, a proto-oncoprotein, a milk protein (e.g. caseins, lactalbumin, whey, and the like), a muscle protein (e.g. myosin, tropomyosin, and the like), a neuroactive peptide (e.g. enkephalins), a tumour growth suppressing protein or peptide, for example, angiostatin or endostatin, both of which inhibit angiogenesis, an anti-sepsis peptide, such as bactericidal permeability-increasing protein (BPI) or an autoantigen, such as collagen, preferably type I or type III collagen, type II collagen, myelin basic protein, myelin proteolipid protein, interphotoreceptor binding protein, acetylcholine receptor, an S-antigen, insulin, glutamic acid dehydrogenase, an islet cell-specific antigen or thyroglobulin, or a transplantation antigen, such as an allo- or xeno-transplantation antigen, for example a MHC protein, including MHCI, MHCII or MHCIII. The protein of interest may be a structurally complex protein that contains, for example, disulphide bonds. The proteins of interest may be a monomeric form, a dimerized form or multimeric form. Thus, in aspects, the protein of interest is a monomer, a dimer, a trimer or additional forms of multimers. In aspects, the proteins of interest produced by the methods described herein are structurally equivalent to a natural human protein and are biological active having substantially the same biological activity as the natural human protein.

In aspects, the protein of interest is a cytokine, which may also be referred to as a plantakine when produced in a plant. In aspects, the protein of interest is selected from IL-37, IL-38, IL-11, IL-38b, IL-33, IL-1Ra, IL-36Ra, IL-2, IL-3, IL-10, CSF3, IL-13, FGF19, CSF23, IL-35, leukemia inhibitory factor (LIF)—an IL-6 class cytokine, IL-4, BMP2, BMP7, TGF-31, or FSH (Table III of Example 1). In aspects, the protein of interest is IL-38. In aspects, the protein of interest is IL-37b. In further aspects, the protein of interest is IL-33. In aspects, the protein of interest is G-CSF. In aspects, the protein of interest is Staphylococcus aureus Protein A.

The heterologous polynucleotide may be codon-optimized. For example, the sequence may be optimised for expression in a plastid and/or plant (e.g., a higher plant).

The protein of interest may be encoded by a polynucleotide comprising (or consisting of) a sequence having at least 80% sequence identity to the sequence defined by any one of SEQ ID NO: 3 to 22. Preferably, the protein of interest is encoded by a polynucleotide comprising (or consisting of) a sequence having at least 70% sequence identity to the sequence defined by SEQ ID NO. 3, SEQ ID NO. 5, SEQ ID NO 6, or SEQ ID NO 12. For example, the protein of interest may be encoded by a polynucleotide comprising (or consisting of) SEQ ID NO. 3, SEQ ID NO. 5, SEQ ID NO 6, or SEQ ID NO 12.

In aspects, the protein of interest is encoded by a polynucleotide having the sequence defined by SEQ ID NO. 3, 5, 6, or 12. In aspects, the protein of interest is encoded by a polynucleotide having the sequence defined by SEQ ID NO. 3 or SEQ ID NO. 5. Some exemplary sequences for each of the proteins of interest (e.g. encoded by each of the genes of interest (GOI)) for use in the transformation vectors/expression cassettes described herein are provided in Example 1. In addition, exemplary methods of synthesizing the genes of interest are provided in Example 1.

In other aspects, the protein of interest can be a variant which maintains substantially the full biological function of the native protein. Thus, the protein of interest can be least 70% identical to a protein encoded by a polynucleotide having the sequence defined by SEQ ID NO. 3, 5, 6, or 12 and wherein the protein of interest retains its biological activity. In other words, the protein of interest can be at least about 70%, at least about 80%, at least about 85%, at least about 90%, at least about 95%, at least about 96%, at least about 97%, at least about 98%, at least about 99% or about 100% identical to a protein encoded by a polynucleotide having the sequence defined by SEQ ID NO. 3, 5, 6, or 12. In aspects, the protein of interest comprises (or consists of) a protein encoded by a polynucleotide having the sequence defined by SEQ ID NO: 3, 5, 6 or 12. In aspects, the protein of interest can be at least about 70%, at least about 80%, at least about 85%, at least about 90%, at least about 95%, at least about 96%, at least about 97%, at least about 98%, at least about 99% or about 100% identical to a protein encoded by a polynucleotide having the sequence defined by SEQ ID NO. 3 or SEQ ID NO. 5. As would be understood, if the protein of interest is 100% identical to the polynucleotide sequence, the protein of interest would not be considered a variant thereof. Thus, the variant protein of interest has an amino acid sequence that is at least about 55%, at least about 60%, at least about 65%, or at least about 70%, typically, at least about 75%, at least about 80%, at least about 85%, at least about 90%, at least about 96%, at least about 97%, at least about 98% or at least about 99% identity to the full-length amino acid sequence of the protein of interest or a fragment thereof. Percent identity between a putative variant and a full-length amino acid sequence is determined using the Blast2 alignment program (Blosum62, Expect 10, standard genetic codes). As would be understood, variations in percent identity can be due, for example, to amino acid substitutions, insertions, or deletions. Amino acid substitutions are defined as one for one amino acid replacements. They are conservative in nature when the substituted amino acid has similar structural and/or chemical properties. Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of polypeptide can be found using computer programs well known in the art, such as DNASTAR software. Whether an amino acid change results in a biologically active protein described herein can readily be determined by assaying for native activity using conventional methods.

### Expression Cassettes and Transformation Vectors

The invention provides an expression cassette comprising a heterologous polynucleotide sequence encoding a protein of interest, wherein the polynucleotide sequence is operably linked to a regulatory sequence; optionally wherein the regulatory sequence is operative in the plastid.

Also provided is an expression cassette, the expression cassette comprising: (a) a heterologous polynucleotide sequence encoding a protein of interest operably linked to a regulatory sequence, (b) a first and a second flanking sequence that allow for integration of the polynucleotide sequence into a plastid genome, wherein one of the flanking sequences is 5′ to the heterologous polynucleotide sequence and other flanking sequence is 3′ to the heterologous polynucleotide sequence; optionally wherein: (i) the regulatory sequence is operative in the plastid, and/or (ii) the first and second flanking sequences allow for the stable integration of the polynucleotide sequence into the plastid genome.

The expression cassette may be comprised within a vector (e.g., a transforming vector). For example, the vector may comprise an expression cassette comprising a heterologous polynucleotide sequence encoding a protein of interest operably linked to a regulatory sequence; optionally wherein the regulatory sequence is operative in the plastid. The vector may further comprise a first and a second flanking sequence that allow for stable integration of the heterologous polynucleotide sequence coding for the protein of interest into the plastid genome, wherein one of the flanking sequences is 5′ to the expression cassette and the other flanking sequence is 3′ to the expression cassette.

Any and all disclosure herein in relation to expression cassettes of the invention may be applied equally and without reservation to vectors of the invention. Similarly, any and all disclosure herein in relation to vectors of the invention may be applied equally and without reservation to expression cassettes of the invention.

Described herein are expression cassettes encoding the proteins of interest described herein, wherein the expression cassette is carried by a transformation vector competent for integrating the expression cassette into a plastid genome. In aspects, the transformation vector comprises the expression cassette, as operably linked components, which comprises a regulatory sequence operative in the plastid, a heterologous polynucleotide sequence coding for the protein described herein, and flanking each side of the expression cassette are flanking DNA sequences which are homologous to a DNA sequence of the target plastid genome. Stable integration of the heterologous polynucleotide coding sequence into the plastid genome of the target plant is typically facilitated through homologous recombination of the flanking sequences with the homologous sequences in the target plastid genome. In other words, the transformation vector described below is component for stably integrating into the plastid genome of the plant described below for expression of the proteins of interest described herein.

The plastid genome can be from any plastid from a plant or algae. The term “plastid” as used herein includes: chloroplasts; chromoplasts, which are present in the fruits, vegetables, and flowers; amyloplasts which are present in tubers such as potato; proplastids, in the roots of higher plants; leucoplasts and etioplasts, both of which are present in the non-green parts of plants, and the plastids of such organisms as algae, which contain plastids. In other words, the plastid can be, for example, a chloroplast, a chromoplast, an amyloplast, a proplastid, a leucoplast or an etioplast. In aspects, the plastid is a chloroplast. In aspects, the plant plastid comprising the plastid genome is transformed with the heterologous polynucleotide sequence coding for the protein of interest, and integrated into the plastid genome such that the protein of interest is expressed in and present in the plastid is provided herein. The heterologous polynucleotide sequence coding the protein of interest is also referred to as the GOI (see above).

The plant can be a dicotyledonous plant, such as tobacco, tomato, soybean or spinach, or the plant can be a monocotyledonous plant, such as maize or rice. Plants transformed in accordance with the present invention may be monocots or dicots and include, but are not limited to, maize, wheat, barley, rye, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugarbeet, sunflower, rapeseed, clover, tobacco, carrot, cotton, alfalfa, rice, potato, eggplant, cucumber, Arabidopsis, turfgrasses, ornamentals and woody plants such as coniferous and deciduous trees. Once a desired gene has been transformed into a particular plant species, it may be propagated in that species or moved into other varieties of the same species, particularly including commercial varieties, using traditional breeding techniques. Also included in the present invention are edible algae, such as unicellular green algae (e.g. Chlamydomonas), multicellular green algae (e.g. Ulva), unicellular red algae (e.g. Porphyridium) and multicellular red algae (e.g. Porphyra), which contain plastid genomes substantially similar to those of higher plant that may be transformed in a similar manner.

In aspects, the plant is a dicotyledonous plant, such as a tobacco plant. In aspects, the tobacco plant is a low-alkaloid tobacco plant, such as, for example, low-alkaloid cultivar 81V9. Examples of suitable low-alkaloid tobacco plants can be found in, for example, Menassa, R., Nguyen, V., Jevnikar, A. and Brandle, J. (2001) A self-contained system for the field production of plant recombinant interleukin-10. Mol. Breeding, 8, 177-185, hereby incorporated by reference. In other words, the low-alkaloid tobacco plant can be used for the expression of the proteins described herein. The present invention is also related to the seed for such a plant, which seed is optionally treated (e.g. primed or coated) and/or packaged (e.g. placed in a bag or other container with, for example, instructions for use). The invention also pertains to plant parts, plant material, plant matter, and cells of plants, wherein the plant parts, plant material, plant matter, and cells of the plant are capable of producing the protein described herein. The present invention is also applicable to the progeny of the plants described herein.

The transformation vectors and/or expression cassettes of the invention for use in stably transforming the plastid genome described herein can be constructed with different regulatory sequences, selectable markers and flanking sequences suitable for integration into a variety of plant plastid genomes.

The regulatory sequence operative in the plastid typically comprises or consists of a promoter to drive transcription of the protein encoded by the expression cassette. Any suitable functional promoter can be used in the vector described herein, for example, the promoter can be a cipP promoter, a 16S rRNA gene promoter, a psbA promoter, a rbcL promoter or a transactivator-mediated promoter regulated by a nuclear transactivator (e.g., the T7 gene 10 promoter when the transactivator is T7). Preferred plastid promoters include the 16S rRNA promoter (Prrn16) and the psbA promoter (PpsbA). The sequences of the typical promoters used in the vector are provided below. Promoters may be modified to, for example, increase their activity. Promoters may also be truncated promoters such as a core promoter. Promoters may be modified to, for example, increase their activity. Promoters may also be truncated promoters such as a core promoter. For example, the psbA promoter may be a core psbA promoter (SEQ ID NO: 34), the Prin promoter may be a core Prrn promoter or the PrbcL promoter may be a core PrbcL promoter (SEQ ID NO: 36). In aspects, the constitutive 16S rRNA core promoter, which can be recognized by the chloroplast encoded RNA polymerase is used to regulate transcription. In other aspects, the promoter is a mutated 16S rRNA promoter with reduced homology to the endogenous 16S rRNA promoter yet with substantially equal functionality. The promoter may be a modified Prm core promoter (SEQ ID NO: 35). The selectable marker gene can be any gene suitable for the methods described herein.

In aspects, the selectable marker gene is the bacterial aadA (aminoglycoside adenyl transferase conferring resistance to spectinomycin) gene. Expression of the aadA gene confers resistance to spectinomycin and streptomycin, and thus allows for the identification of plant cells expressing this marker. The aadA gene product allows for continued growth and greening of cells whose chloroplasts comprise the selectable marker gene product. Thus, in aspects, the aadA gene conferring spectinomycin resistance can be used for selection of transgenic shoots. As would be understood, the use of antibiotic containing growth media permits selection of only those cells that have incorporated the foreign genes. Once transgenic plants are regenerated, antibiotic resistance genes serve no useful purpose, but they continue to produce their gene products. Thus, in some aspects, the selectable marker gene is an antibiotic free selectable marker, such as for example, a plant specific enzyme like betaine aldehyde dehydrogenase (BADH). In this way, integration of the foreign genes into the plastid genome can be evaluated by the function of the expressed enzyme from the foreign integrated genes. In some aspects, the selectable marker used for selection of the transformed plastids in the plant cells constitutes one or a few point mutations in the endogenous plastid genes that confer tolerance to specific antibiotics or herbicides. These point mutations can be introduced into the plastome by specifically designed transformation vectors, as would be understood in the art.

The vectors and/or expression cassettes of the invention are designed to integrate into a plastid genome. In other words, the vectors and/or expression cassettes of the invention are capable of integration into a plastid genome. For example, the vectors and/or expression cassettes may be designed to integrate the heterologous polynucleotide sequence into an intergenic region between adjacent plastid genes. As such, the vectors and/or expression cassettes may comprise a first sequence (a first flanking sequence) that is homologous to a first sequence from a plastid genome and a second sequence (a second flanking sequence) that is homologous to a second sequence from a plastid genome. In other words, the first flanking sequence may comprise a first sequence from a plastid genome and the second flanking sequence may comprise a second sequence from a plastid genome. The first flanking sequences may be 5′ to the heterologous polynucleotide sequence and second flanking sequence may be 3′ to the heterologous polynucleotide sequence;

The flanking regions may be further defined as a first flanking region and a second flanking region, and the flanking regions are used for integration into the plastid genome by homologous recombination. In aspects, the flanking DNA sequences are substantially or fully homologous to sequences flanking the cassette integration site in the plastid genome.

Any suitable flanking sequence can be used. In aspects, the flanking sequences comprises trl and trnA genes (chloroplast transfer RNAs genes for Isoleucine and Alanine, respectively) such that the flanking sequences are used for homologous recombination to insert the heterologous polynucleotide sequence into the spacer region in the chloroplast genome. In some aspects, the flanking sequences comprise rps12 and tmnV genes. In some aspects, the flanking sequences comprise tmnT and trnG genes. Any one of the pairs described above for the flanking regions may be referred to as the first flanking region or the second flanking region. In aspects, the first and second flanking sequences provide for homologous recombination to insert the heterologous polynucleotide coding for the protein described herein into the spacer region in an inverted repeat region of a chloroplast genome. Integration of the heterologous polynucleotide into the inverted repeat region increases the transgene copy number, for example, by doubling the transgene copy number. The plastid transformation vector may optionally comprise at least one chloroplast origin of replication.

Preferably, the first and second flanking sequence are engineered such that integration of the vector into the plastid genome does not result in deletion of a portion of the plastid genome that is essential for survival or fitness. Preferably, the first and second flanking sequence are homologous to adjacent sequences in the plastid genome. By adjacent sequences, it is meant that the there are not intervening coding sequences between the two plastid sequences.

The flanking sequences may comprise (or consist of) intergenic/non-transcribed sequences of the plastid genome, coding sequence or combinations thereof. When a flanking sequence comprises a coding sequence, the flanking sequence will typically comprise an entire gene or exon.

Each of the flanking sequences may have 100% sequence identity to plastid genome sequences. Alternatively, each flanking sequence may have at least 80% sequence identity, at least 90% sequence identity, at least 95% sequence identity, at least 98% sequence identity to a plastid genome sequence. For example, a flanking sequence may have at least 95% sequence identity to a plastid genome sequence.

Flanking sequences may be of any length, so long as they are able to facilitate integration of the vector into the plastid genome. Each flanking sequence may comprise (or consist of) a sequence of at least 500 base pairs, at least 750 base pairs, at least 1000 base pairs, at least 1250 base pairs, at least 1500 base pairs, at least 1750 base pairs, at least 2000 base pairs, at least 2250 base pairs, at least 2500 base pairs, at least 2750 base pairs, or at least 3000 base pairs from a plastid genome. Each flanking sequence may comprise (or consist of) a sequence of from 500 to 3000 base pairs, from 750 to 3000 base pairs, from 1000 to 2750 base pairs, from 1250 to 2500 base pairs, or from 1500 to 2250 base pairs.

Flanking sequences may comprise (or consist of) a sequence from the tobacco plastome (NC_001879), tomato plastome (NC_007898) or the Chrysanthemum indicum plastome (NC_020320). For example, the flanking sequences may have at least 95% sequence identity to a sequence of from 750 to 3000 base pairs within the tobacco plastome (NC_001879), tomato plastome (NC_007898) or the Chrysanthemum indicum plastome (NC_020320).

Pairs of flanking sequences may be selected based on their proximity in the plastid genome. For example, pairs of flanking sequences may comprise (or consist of) sequences from adjacent genes.

The first flanking sequence may comprise or consist of a trnl, a rps12 or a tmnT or one or more fragments thereof. The rnl, rps12 or trnT may be a gene, an intron or a fragment thereof. For example, the fragments may be any fragment which function to enable the insertion of the heterologous polynucleotide into the integration site of the plastid genome by homologous recombination. The first flanking sequence may comprise or consist of trnl or a fragment thereof. The first flanking sequence may comprise or consist of rps12 or a fragment thereof. The first flanking sequence may comprise or consist of trnT or a fragment thereof.

The second flanking sequence may comprise or consist of a trnA, a trnV, or a trnG or one or more fragments thereof. The trnA, trnV, or trnG may be a gene, an intron, or a fragment thereof. For example, the fragments may be any fragment which function to enable the insertion of the heterologous polynucleotide into the integration site of the plastid genome by homologous recombination. The second flanking sequence may comprise or consist of trnA or a fragment thereof. The second flanking sequence may comprise (or consist of) trnV or a fragment thereof. The second flanking sequence may comprise (or consist of) trnG or a fragment thereof. For example, the trG fragment may be trnG exon 2.

The vector may comprise a pair of flanking sequences selected from: a tml sequence and a trnA sequence, a trnl sequence and a trnV sequence, a trnl sequence and a trnG sequence, a rps12 sequence and a trnA sequence, a rps 12 sequence and a trnV sequence, a rps12 sequence and a trnG sequence, a trnT sequence and a tmnA sequence, a trnT sequence and a trV sequence, and a tmnT sequence and a trnG sequence.

In some embodiments, the first and second flanking sequences are engineered such that, following transformation, the heterologous polynucleotide encoding the protein of interest is inserted in the intergenic region between either: (i) the trnl and trnA genes of the rrn16 operon, (ii) the rps 12 and trnV genes of the tobacco plastome (NC_001879) or tomato plastome (NC_007898), or (iii) the trnT and trnG genes of the Chrysanthemum indicum plastome (NC_020320). Preferably, the following transformation, the heterologous polynucleotide encoding the protein of interest is inserted in the intergenic region between the trnl and trnA genes of the rm16 operon.

In some embodiments, the first flanking sequence comprises (or consists of) a trnl gene, or fragment thereof, and the second flanking sequence comprises (or consists of) a trnA gene, or fragment thereof. Preferably, the first flanking sequence comprises the trnl gene and the second flanking sequence comprises the trnA gene. The sequences of the trnl and tmnA genes can be determined by a person of skill in the art, for example, with reference to the tobacco plastome (NC_001879) or tomato plastome (NC_007898).

The first flanking sequence may comprise or consist of nucleotides 104,553 to 105,331 of the tobacco plastome (NC_001879). For example, the first flanking sequence may comprise or consist of nucleotides 103473 to 105395 of the tobacco plastome (NC_001879).

The second flanking sequence may comprise or consist of nucleotides 105,396 to 106,177 of the tobacco plastome (NC_001879). For example, the second flanking sequence may comprise or consist of nucleotides 105396 to 106485 of the tobacco plastome (NC_001879).

The first flanking sequence may comprise or consist of nucleotides 104,553 to 105,331 and the second flanking sequence may comprise or consist of nucleotides 105,396 to 106,177 (both of the tobacco plastome. NC_001879). The first flanking sequence may comprise or consist of nucleotides 103473 to 105395 and the second flanking sequence may comprise or consist of nucleotides 105396 to 106485 (both of the tobacco plastome, NC_001879).

In some embodiments, the first flanking sequence comprises or consists of a rps12 gene, or fragment thereof, and the second flanking sequence comprises or consists of a trnV gene, or fragment thereof. Preferably, the first flanking sequence comprises the rps12 gene and the second flanking sequence comprises the trnV gene. The sequences of the rps 12 and trnV genes can be determined by a person of skill in the art, for example, with reference to the tobacco plastome (NC_001879) or tomato plastome (NC_007898).

The first flanking sequence may comprise or consist of nucleotides 100,624 to 100,855 of the tobacco plastome (NC_001879). For example, the first flanking sequence may comprise or consist of nucleotides 100,162 to 101,709 of the tobacco plastome (NC_001879).

The second flanking sequence may comprise or consist of nucleotides 102,463 to 102,534 of the tobacco plastome (NC_001879). For example, the second flanking sequence may comprise or consist of nucleotides 101710 to 103183 of the tobacco plastome (NC_001879).

The first flanking sequence may comprise or consist of nucleotides 100,624 to 100,855 and the second flanking sequence may comprise or consist of nucleotides 102,463 to 102,534 (both of the tobacco plastome, NC_001879). The first flanking sequence may comprise or consist of nucleotides 100162 to 101709 and the second flanking sequence may comprise or consist of nucleotides 101710 to 103183 (both of the tobacco plastome, NC_001879). In some embodiments, the first flanking sequence comprises or consists of a trnT gene, or fragment thereof, and the second flanking sequence comprises or consists of the trnG gene, or fragment thereof. Preferably, the first flanking sequence comprises the trnT gene and the second flanking sequence comprises exon 2 of the trnG gene. The sequences of trnT and trnG gene/exon can be determined by a person of skill in the art, for example, with reference to the Chrysanthemum indicum plastome (NC_020320).

The first flanking sequence may comprise or consist of nucleotides 30.891 to 30,958 of the tobacco plastome (NC_001879). For example, the first flanking sequence may comprise or consist of nucleotides 30,775 to 32,087 of the Chrysanthemum indicum plastome (NC_020320).

The second flanking sequence may comprise or consist of nucleotides 30,702 to 30,724 of the tobacco plastome (NC_001879). For example, the second flanking sequence may comprise or consist of nucleotides 29,266 to 30,774 of the Chrysanthemum indicum plastome (NC_020320).

The first flanking sequence may comprise or consist of nucleotides 30,891 to 30,958 and the second flanking sequence may comprise or consist of nucleotides 30,702 to 30.724 (both of the Chrysanthemum indicum plastome, NC_020320). The first flanking sequence may comprise or consist of nucleotides 30,775 to 32,087 and the second flanking sequence may comprise or consist of nucleotides 29,266 to 30,774 (both of the Chrysanthemum indicum plastome, NC_020320).

The expression cassette and/or vector may further comprise a heterologous spacer sequence. The skilled person will appreciate that genes in plastid genomes are typically arranged in multi-gene clusters, called operons. The DNA sequence elements that separate the genes in an operon are called spacers. Usually situated under a singular genetic promoter, operon genes are transcribed as a polycistronic mRNA molecule, including the spacer sequences. As a non-limiting example, a spacer may be the DNA element that separates psbN and psbH genes in an operon (otherwise known as a psbN-psbH spacer). Spacer sequences may be attributed with several biological functions, mostly related to mRNA stability, processing and efficient translation.

Different heterologous spacer sequences can be implemented in the design of an expression cassette or vector. Heterologous spacer sequences may be selected based on their low sequence identity to endogenous plastome elements (for example, to reduce the possibility of deleterious recombination events after plastome transformation). For example, the heterologous spacer sequence may have less than 80% sequence identity, less than 70% sequence identity, less than 60% sequence identity, or less than 50% sequence identity to an endogenous spacer sequence within the plastome. Preferably, the heterologous spacer sequence has low sequence identity to an endogenous spacer sequence found adjacent to or in close proximity to the insertion site. In other words, a heterologous spacer sequence may not have high sequence identity to an endogenous spacer sequence found adjacent to or near the insertion site (i.e., as determined by the flanking sequences). For example, a heterologous spacer sequence may not have more than 80% sequence identity, more than 90% sequence identity or 100% sequence identity to an endogenous spacer sequence adjacent to or near to the insertion site. As an example, a heterologous spacer sequence may not have more than 80% sequence identity to an endogenous spacer sequence within 1 kb, 2.5 kb, 5 kb, or 10 kb of the insertion site (i.e., as determined by the flanking sequences). Additionally (or alternatively) a heterologous spacer sequences may be selected if, following transformation, the heterologous spacer sequence and the endogenous spacer sequence are in different orientations. The expression cassette and/or vector may also comprise spacer regions that can be between about 50 and about 80 base pairs in length, and are typically homologous to nucleotides found between the genes psbN and psbH, or rps2 and atpl, or rpoC2 and rps2 in the plastid genome.

Spacer sequences typically comprise or consist of from 50 to 80 base pairs. Preferably, the spacer sequence comprises or consists of from 50 to 80 contiguous base pairs from a plastid genome (e.g., from a higher plant). Preferably, the spacer sequence comprises or consists of a sequence of from 50 to 80 contiguous base pairs that are conserved among plant species. For example, the spacer sequence may comprise or consist of from 55 to 80 base pairs, from 60 to 80 base pairs, from 65 to 80 base pairs, from 70 to 80 base pairs or from 75 to 80 base pairs. Exemplary spacer sequences may comprise or consist of the sequences found between the psbN and psbH genes, between the rps2 and atpl genes, or between the rpoC2 and rps2 genes in a plastid genome. For example, the spacer sequence may comprise or consist of a sequence of from 50 to 80 contiguous base pairs from between the psbN and psbH genes of the plastid genome. The spacer sequence may comprise or consist of a sequence of from 50 to 80 contiguous base pairs from between the rps2 and atpl genes of the plastid genome. The spacer sequence may comprise or consist of a sequence of from 50 to 80 contiguous base pairs from between the rpoC2 and rps2 genes of the plastid genome. The spacer may also comprise or consist of a sequence having at least 70% sequence identity, at least 80% sequence identity, at least 90% sequence identity or at least 95% sequence identity to from 50 to 80 contiguous base pairs from between the psbN and psbH genes, between the rps2 and atpl genes, or between the rpoC2 and rps2 genes in a plastid genome.

The spacer may comprise or consist of a polynucleotide sequence having at least 70% sequence identity, at least 80% sequence identity, at least 90% sequence identity or at least 95% sequence identity to SEQ ID NO: 23 (i.e., nucleotides 13630-13702 found between the genes atpH and atpF in the tobacco plastome (NC_001879)), SEQ ID NO: 24 (i.e., nucleotides 77030-77095 found between the genes psbN and psbH in the tobacco plastome (NC_001879)), SEQ ID NO: 25 (i.e., nucleotides 16043-16115 found between the genes rps2 and atpl in the tobacco plastome (NC_001879)), SEQ ID NO: 26 (i.e., nucleotides 16943-17015 found between the genes rpoC2 and rps2 in the tobacco plastome (NC_001879)). For example, the spacer may comprise of consist of one of SEQ ID NO: 23, SEQ ID NO: 24, SEQ ID NO: 25 or SEQ ID NO: 26.

The expression cassette and/or vector may further comprise a 5′ untranslated region (UTR). 5′ UTR sequences may provide regulatory control of expression of a protein of interest. In particular, the 5′ UTR may be capable of providing transcription and/or translation enhancement of the heterologous polynucleotide coding for the protein of interest. Preferably, the 5′ UTR may be any 5′ UTR which enhances transcription and/or translation of the heterologous polynucleotide in a plastid. The regulatory sequence may further comprise a 5′ UTR, as defined herein. Exemplary 5′ UTRs include those of the plastid psbA gene, rbcL and the bacteriophage T7gene 10 (17G10). For example, the 5′ UTR may be selected from SEQ ID NO. 27. SEQ ID NO: 28 or SEQ ID NO: 29. The 5′ UTR may comprise or consist of a sequence having at least 70% sequence identity, at least 80% sequence identity, at least 90% sequence identity or at least 95% sequence identity to SEQ ID NO. 27, SEQ ID NO: 28 or SEQ ID NO: 29.

The expression cassette and/or vector may also further comprise a plastid gene 3′ untranslated sequence (3′ UTR) sequence, which is useful to increase the stability of the transcript. The role of untranslated sequences is preferably to direct the 3′ processing of the transcribed RNA rather than termination of transcription. In aspects, the 3′ UTR is a plastid rps16 gene 3′ untranslated sequence or the Arabidopsis plastid psbA gene 3′ untranslated sequence. The 3′ end of the psbC gene, the rbcL gene or the psbA gene are typically selected for the untranslated 3′ sequences. Exemplary 3′ UTR sequences may be selected from SEQ ID NO: 30 to 33. The 3′ UTR may comprise or consist of a sequence having at least 70% sequence identity, at least 80% sequence identity, at least 90% sequence identity or at least 95% sequence identity to SEQ ID NO: 30, SEQ ID NO: 31, SEQ ID NO: 32 or SEQ ID NO: 33.An expression cassette and/or vector of the invention may comprise both a 5′ UTR and a 3′ UTR. Thus, an expression cassette comprising a 3′ UTR as defined herein may also further comprise a 5′ untranslated sequence (5′ UTR) sequence which is useful to enhance translation. Any combination of 5′ UTR and 3′ UTR may be used according to the invention. For example, the 5′ end of the psbA gene, phage T7 G10 gene, or rbcL genes may typically be selected for the untranslated 5′ sequences. In aspects, the transformation vectors contain the psbA 5′ UTR as a cis-acting regulatory element, controlling the translation of genes in higher plants. In other aspects, the 5′ UTR is a 5′ UTR of T7G10 gene.

The expression cassette/vector may comprise in the 5′ to 3′ direction: a first flanking sequence (e.g., a trnl sequence), a spacer (e.g., a psbN-psbH spacer), a promoter sequence (e.g., , a core promoter such as a PpsbA core promoter), a 5′UTR (e.g., a psbA 5′UTR), a gene of interest/heterologous polynucleotide encoding a protein of interest (e.g., IL-37b) and a 3′ UTR (e.g., a TrbcL 3′UTR). The expression cassette/vector may further comprise a Shine Dalgarno sequence; optionally wherein the Shine Dalgarno sequence is 5′ to the promoter sequence; further optionally wherein the Shine Dalgarno sequence is immediately 5′ to the promoter sequence. The expression cassette/vector may further comprise a polynucleotide sequence encoding a selection marker. Preferably, the selection marker is operably linked to the promoter sequence. Preferably, both the selection marker and the gene of interest are operably linked to the promoter. The expression cassette/vector may further comprise a lumen target sequence. Preferably, the expression cassette/vector comprises in the 5′ to 3′ direction: a first flanking sequence, a spacer sequence, a Shine-Dalgarno sequence, a selection marker, a core promoter sequence, a 5′ UTR sequence, a heterologous polynucleotide sequence encoding a protein of interest, a 3′ UTR sequence, and a second flanking sequence. Preferably the 5′UTR is selected from psbA or T7G10. Preferably the heterologous polynucleotide sequence comprises or consists of a gene encoding IL-38, IL-37b, IL-33, G-CSF or protein A of Staph aureus. Specific aspects of the present invention are disclosed below.

In particular, the following legend provides clarity on the specific sequences. In these specific aspects, the vector backbone was (pUC57 Amp^{R}):
Grey (GY)-Flanking sequences
Yellow (Y)-Spacer sequences
Green (GN)-Shine-Dalgarno sequence
Dark Blue (DB)-Core promoter sequences
Light Blue (LB)-aadA sequence
Blue-Green (BG)-5′UTR sequences
Brown (BN)-Lumen-targeting sequences
Purple (PU)-GOI sequences
Pink (PK)-3′UTR sequences

In specific aspects, the transformation vector described herein comprises the following components, operably linked, so that the vector can be used to stably transform the plastid and produce the recombinant proteins described herein. In specific aspects, for IL-38 production described herein, the transformation vector comprises the following components-trnl-^{GY}-psbN-psbH-^{Y}-^{GN}-aadA-^{LB}-TpsbC-^{PK}-PpsbA-^{DB}_T7G10-^{GB}-IL-38-^{PU}-TrbcL-^{PK}-trnA-^{GY}. In specific aspects, for IL-37b production described herein, the transformation vector comprises the following components:-trnl-^{GY}-psbN-psbH-^{Y}-^{GN}-aadA-^{LB}-TpsbC-^{PK}-PpsbA-^{DB}-psbA-^{BG}-IL-37b-^{PU}-TrbcL-^{PK}-trnA-^{GY}. In specific aspects, for IL-33 production described herein, the transformation vector comprises the following components:-trnl-^{GY}-psbN-psbH-^{Y}-^{GN}-aadA-^{LB}-TpsbC-PK-PpsbA-^{DB}-psbA-^{BG}-IL-33-^{FU}-TrbcL-^{PK}-trnA-^{GY}. In specific aspects, for G-CSF production described herein, the transformation vector comprises the following components: trnl-^{GY}-psbN-psbH-^{Y}-^{GN}-aadA-^{LB}-TpsbC-^{PK}-PpsbA-^{DB}-psbA-^{BG}-G-CSF-^{PU}-TrbcL-^{PK}-trnA-^{GY}. In specific aspects, for protein A production described herein, the transformation vector comprises the following components:-trnl-^{GY}-psbN-psbH-^{Y}-^{GN}-aadA-^{LB}-TpsbC-^{PK}-PpsbA-^{DB}-T7G10-^{BG}-stpA-^{PU}-TrbcL-^{PK}-trnA-GY. In each of the above aspects, the function of the components is indicated by the superscript annotation, as defined above.

In aspects, a transformation vector for stably transforming a plastid, comprising, an expression cassette, comprising, as operably-linked components, a promoter comprising psbA operative in the plastid, a heterologous polynucleotide encoding IL-38, and, flanking each side of the expression cassette, a first DNA flanking sequence comprising trnl and a second flanking

DNA sequence comprising tmnA, which allow for stable integration of the heterologous polynucleotide sequence encoding IL-38 into the plastid genome. In another specific aspect, a transformation vector for stably transforming a plastid, comprising, an expression cassette, comprising, as operably-linked components, a promoter comprising psbA operative in the plastid, a heterologous polynucleotide encoding protein A of Staph aureus, and, flanking each side of the expression cassette, a first DNA flanking sequence comprising trnl and a second flanking DNA sequence comprising tmA which allow for stable integration of the heterologous polynucleotide sequence encoding protein A of Staph aureus into the plastid genome. In these specific aspects, the spacer region is between psbN and psbH genes, the 5′UTR comprises T7G10, the 3′ UTR comprises psbC, and the selectable marker comprises aadA. Examples of these transformation vectors are provided in Example 3 or 10, respectively.

In aspects, a transformation vector for stably transforming a plastid, comprising, an expression cassette, comprising, as operably-linked components, a promoter comprising a psbA gene operative in the plastid, a heterologous polynucleotide encoding IL-37b, and, flanking each side of the expression cassette, a first DNA flanking sequence comprising tml and a second flanking DNA sequence comprising trA which allow for stable integration of the heterologous polynucleotide sequence encoding IL-37b into the plastid genome. In this specific aspect, the spacer region is between psbN and psbH genes, the 5′UTR comprises psbA, the 3′ UTR comprises psbC and the selectable marker comprises aadA. An example of such a transformation vector is provided in Example 3.

In aspects, a transformation vector for stably transforming a plastid, comprising, an expression cassette, comprising, as operably-linked components, a promoter comprising psbA operative in the plastid, a heterologous polynucleotide encoding IL-33, and, flanking each side of the expression cassette, a first DNA flanking sequence comprising trl and a second flanking DNA sequence comprising trnA which allow for stable integration of the heterologous polynucleotide sequence encoding IL-33 into the plastid genome. In this specific aspect, the spacer region is between psbN and psbH genes, the 5′ untranslated region (UTR) comprises psbA, the 3′ UTR comprises psbC and the selectable marker comprises aadA. An example of such a transformation vector is provided in Example 3.

In aspects, a transformation vector for stably transforming a plastid, comprising, an expression cassette, comprising, as operably-linked components, a promoter comprising a psbA gene operative in the plastid, a heterologous polynucleotide encoding G-CSF, and, flanking each side of the expression cassette, a first DNA flanking sequence of trnl and a second flanking DNA sequence comprising tmnA which allow for stable integration of the heterologous polynucleotide sequence encoding G-CSF into the plastid genome. In this specific aspect, the spacer region is between psbN and psbH genes, the 5′UTR comprises psbA, the 3′ UTR comprises psbC and the selectable marker comprises aadA. An example of such a transformation vector is provided in Example 3.

Reference to genetic sequences herein refers to single- or double-stranded nucleic acid sequences and comprises a coding sequence or the complement of a coding sequence for protein of interest. Degenerate nucleic acid sequences encoding proteins of interest, as well as homologous nucleotide sequences which are at least about 50%, at least about 55%, at least about 60%, at least about 65%, at least about 60%, typically at least about 75%, at least about 90%, at least about 96%, or at least about 98% identical to the cDNA may be used in accordance with the teachings herein polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologs, and variants of nucleic acid sequences which encode biologically active proteins of interest also are useful polynucleotides.

Variants and homologs of the nucleic acid sequences described above also are useful nucleic acid sequences. Typically, homologous polynucleotide sequences can be identified by hybridization of candidate polynucleotides to known polynucleotides under stringent conditions, as is known in the art. For example, using the following wash conditions: 2×SSC (0.3M NaCl, 0.03M sodium citrate, pH 7.0), 0.1% SDS, room temperature twice, 30 minutes each; then 2×SSC, 0.1% SOS, 50° C. once, 30 minutes; then 2×SSC, room temperature twice, 10 minutes each homologous sequences can be identified which contain at most about 25-30% base pair mismatches. More preferably, homologous nucleic acid strands contain 15-25% base pair mismatches, even more preferably 5-15% base pair mismatches. Stringent wash conditions are well known and understood in the art and are disclosed, for example, in Sambrook et al., MOLECULAR CLONING: A LABORATORY MANUAL, 2nd ed., 1989, at pages 9.50-9.51, the disclosure of which is incorporated herein by reference.

### Methods Involving, and Uses, of the Proteins of Interest

In aspects, methods of transforming plastid genomes with the transformation vector described herein, to express the protein of interest described herein are provided, as well as to the transformed plants, seeds, plant parts, plant cells and progeny thereof. In particular, the invention provides a method for producing a protein of interest comprising: integrating the transformation vector or expression cassette of the invention into a plastid genome of a plant cell; and growing the plant cell to thereby express the protein of interest.

Methods of chloroplast isolation and transformation are generally understood in the art. For example, chloroplasts may be isolated from crude homogenates by centrifugation (1500×g). This fraction is typically free of other cellular proteins. Isolated chloroplasts can be burst open by osmotic shock to release foreign proteins that are compartmentalized in this organelle along with few other native soluble proteins. Moreover, it was possible to introduce isolated intact chloroplasts into protoplasts and regenerate transgenic plants. However, after the discovery of the gene gun as a transformation device, it was possible to transform plant chloroplasts without the use of isolated plastids and protoplasts. Plastid transformation technology is described extensively in U.S. Pat. Nos. 5,451,513, 5,545,817, 5,545,818 and 5,576.198; in PCT Patent Application Publication Nos. WO 95/16783 and WO 97/32977; and in McBride et al., Proc. Natl. Acad. Sci. USA 91:7301-7305 (1994), all of which are incorporated herein by reference.

In aspects, transformation of chloroplasts is carried out with particle bombardment to introduce transgenes into leaf chloroplasts. Stable transformation preferably involves uniform conversion of all plastome copies. Securing genetically stable lines of plants with transgenic chloroplast typically requires every chloroplast to carry the inserted gene. This homoplastomic state is achieved through amplification and sorting of transgenic chloroplasts with the elimination of the wild-type copies on selective medium (e.g. an antibiotic containing medium). Thus, a method of the invention may comprise one or more step to amplify and/or sort transgenic plastids (e.g. chloroplasts), and optional the use of a selective medium (e.g. an antibiotic containing medium) to eliminate wild-type copies of the plastid (e.g. chloroplast).

The integration of cloned plastid DNA into the plastid genome typically occurs through site-specific homologous recombination in plants, such as, for example, in tobacco (N. tabacum) and can exclude the foreign vector DNA. In aspects, chloroplast transformation utilizes two flanking sequences that, through homologous recombination, insert foreign DNA into the spacer region between the functional genes of the chloroplast genome, thereby targeting the foreign genes to a precise location. This eliminates the position effects and gene silencing frequently observed in nuclear transgenic plants. As would be understood, southern blots assays could be used to confirm stable integration of foreign genes into all of the chloroplast genomes (10,000 copies per cell) resulting in homoplastomy. Aspects such as these would limit public concerns or perception of genetically modified crops, and would be helpful in the development of edible pharmaceutically relevant proteins, using the methods described herein.

In aspects, plastome (e.g. plastid genome) transformation and regeneration of transplastomic clones is carried out by standard biolistic procedures (Svab, Z. and Maliga, P. (1993) High frequency plastid transformation in tobacco by selection for a chimeric aadA gene. Proc. Natl Acad. Sci. USA, 90, 913-917; Lutz, K. A. and Maliga, P. (2007) Transformation of the plastid genome to study RNA editing. Methods Enzymol. 424, 501-518; each of which is hereby incorporated by reference). In general, the plants are grown aseptically by germination of seeds in an appropriate medium and fully expanded, dark green leaves of about two months old plants, can be used for the bombardment. For the bombardment, it is typical to place the leaves abaxial side up and use tungsten or gold microprojectiles that are coated with plasmid DNA (chloroplast transformation vectors) and bombardments carried out using a suitable biolistic device (e.g. PDS1000/He (BioRad)). Following this, in aspects, the leaves are incubated at a temperature under a photoperiod. In aspects, the temperature is about 25° C. and the photoperiod is about 12 hours. Following a few days after the bombardment, the leaves are typically chopped into small pieces and placed on the selection medium (e.g. medium containing spectinomycin dihydrochloride) with the abaxial side touching the medium. In aspects, the regenerated spectinomycin resistant shoots are then chopped into small pieces and subcloned into petri dishes containing the same selection medium. At this point, resistant shoots from the second culture cycle can be transferred to the selective rooting medium (e.g. medium containing spectinomycin dihydrochloride) and rooted plants can be transferred to soil and grown at a temperate under about 16 hours light—about 8 hours darkness conditions. In aspects, the temperature is about room temperature (e.g. about 25° C.).

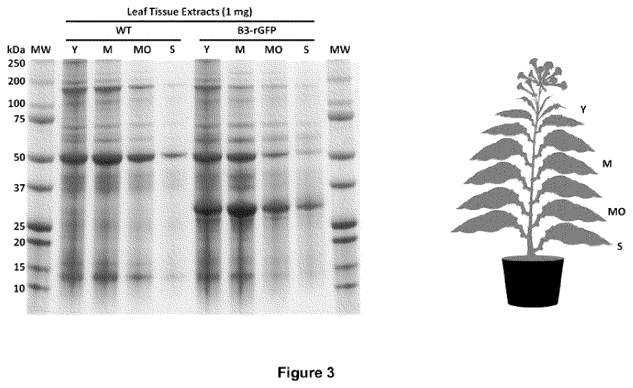

Once the plastid of the plant is transformed and the protein of interest expressed therein, the leaves of the plant can be harvested using conventional methods. Typically, an amount of leaf tissue is harvested from the regenerated clones. In aspects, protein accumulation is measured in young, mature and/or senescing leaves. A young leaf is typically taken from the top five leaves, a mature leaf is typically green and fully-grown from the mid-section of the plant, and the old leaf is typically senescent and from the very bottom of the plant (FIG. 3). In aspects, the methods described herein allow for the manufacture of up to about 4 g of purified recombinant protein per 1 kg of fresh leaf weight. Thus, per 1 kg of fresh leaf weight, the methods described herein can produce about 100 mg, about 250 mg, about 500 mg, about 750 mg about 1 g, about 2 g, about 3 g or about 4 g of protein of interest described herein. In aspects, the protein of interest is present in the plastid in an amount of from about 0.1% TSP to about 60% TSP, including any number in between such as, about 0.5%, about 1%, about 5%, about 10%, about 15%, about 20%, about 25%, about 30%, about 35%, about 40%, about 45%, about 50%, or about 55% TSP. In aspects, the protein of interest is present in an amount of at least 10% TSP. Thus, in aspects, the protein of interest is present in the plastid as a monomer, a dimer, a trimer or additional forms of multimers in amount of about 0.1% TSP to about 60% TSP.

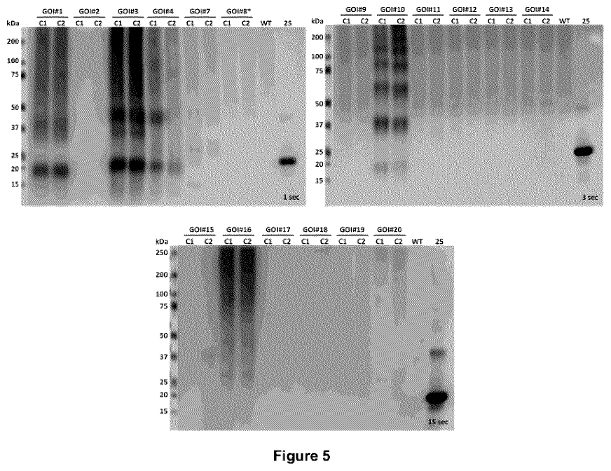

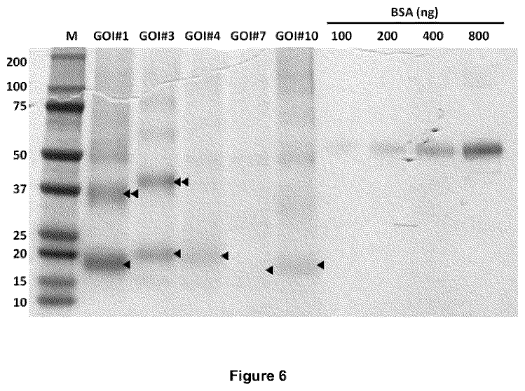

Once the protein of interest is expressed, the expression thereof can be examined using conventional methods, such as for example SDS-PAGE and Western blotting of extracts of soluble proteins (FIGS. 5 and 6). The expressed proteins of interest (GOIs) can also be purified using a variety of different purification tags, such as c-myc or His-tags, such as 7×HIS affinity tag, and the like. In aspects, the expressed proteins are tagged with c-myc and the purified proteins are separated using conventional methods, such as SDS-PAGE and visualized by staining. When the proteins produced by the methods described herein are tagged with, for example, a 7×HIS affinity tag, the tag can be either an N-terminal or C-terminal tag, which typically allows for unhindered purification and physiological activity.

Expression and accumulation levels of the proteins of interest described herein produced in plants described herein can be assessed at any stage of the plant, such as young, mature, or old, as described above. In aspects, the assessment is taken at mature stages of the plant, using conventional methods, such as, for example, SDS-PAGE and Western blotting. Purification of the protein of interest from leaf tissue extracts can also be carried out using an immobilized metal-affinity chromatography (IMAC) procedures. Advantageously, the IMAC procedure can provide a more cost-effective purification procedure, in comparison to, for example, c-myc tag-assisted purification procedures, thus making it, in aspects, the typical method for purification of the proteins produced by the methods described herein.

In aspects, a method for producing a protein of interest is provided. The method comprises integrating the transformation vector described herein into the plastid genome of a plant cell; and growing the plant cell to thereby express the protein of interest. The plastid genome can be from any of the plants described herein. In aspects, the plastid genome of the plant cell is from a low-alkaloid tobacco plant. The method can further comprise recovering the protein of interest, and the recovering can be through, for example, isolating and purifying the protein of interest as described herein. While isolating and purifying the protein of interest, made by the methods described herein, can be done using conventional methods, in aspects, the purifying uses the IMAC procedure described above. Typically, purification results in between about 75% to about 100% protein of interest from the crude extract, and any amount in between, such as about 80%, about 85%, about 90%, or about 95% protein of interest from the extract. In aspects, the purification procedure results in greater than 95% pure protein of interest.

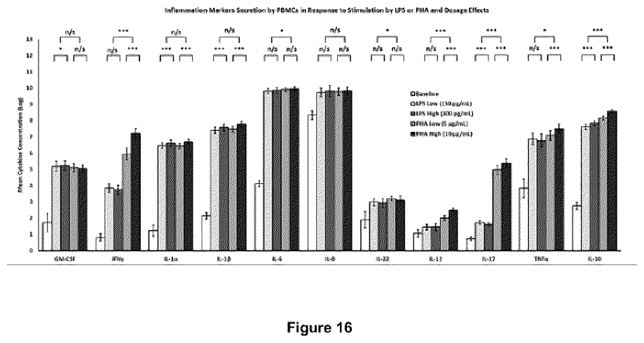

The proteins produced and purified by the methods described herein are capable of modulating an immune response. The immune response may be an ex vivo or in vitro immune response, such as modulating (e.g. generally reducing) the responsiveness of immune cells or tissue cells to a particular stimuli or the immune response can be an in vivo (e.g. in a mammal, such as a human) immune response, such as modulating (e.g. generally reducing) an ongoing inflammatory response in the mammal. Thus, proteins of interest, expressed and purified using the methods described herein are competent to modulate an immune response indicating that the expressed and purified proteins of interest described herein are biologically active and even biologically relevant for, for example, treating an immune-related disease. In aspects, modulating the immune response involves altering the responsiveness of peripheral blood mononuclear cells (PMBCs) stimulated with inflammatory agents (IAs). IAs are any agent that is capable of eliciting an immune response from immune cells, such as, for example, lipopolysaccharide (LPS), phytohemagglutinin (PHA) and the like. Interestingly, incubation of immune cells, such as PBMCs with the IAs results in the enhanced production of an array of cytokine mediators, such as, for example, GM-SCF, IFNγ, TNFα, IL-1α, IL-1β, IL-6, IL-8, IL-22, IL12, IL-17 and IL-10 (see Example 11, FIG. 16). Surprisingly, when the proteins of interest, such as IL-38 and IL-37 produced by the methods described herein, when incubated with the immune cells stimulated with IAs, such as LPS, an anti-inflammatory effect was noted (see, Example 11, Table VII). For example, IL-37 can reduce levels of IFNγ, IL-1α. IL-1β, IL-22. IL-17 and TNFα in LPS-stimulated PBMCs. In this way, IL-37 produced by the methods descried herein may reduce LPS-induced inflammation. These effects may also be dose dependent. Thus, the proteins of interest produced by the methods described herein may alter the cytokine secretion profile of immune cells (e.g. PBMCs) which may have implications for controlling (e.g. attenuating) inflammatory immune responses therefrom.

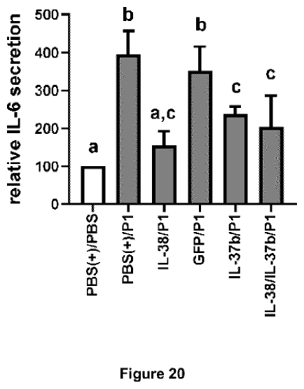

In other aspects, the protein of interest produced and purified by the methods provided herein is competent to modulate an immune response from tissue cells that are stimulated with pathogen or pathogen products. The pathogen or pathogen products could be from bacteria, virus, fungi and the like. For example, the pathogen can be, but is not limited to severe acute respiratory syndrome coronavirus (SARS-COV-2, SARS-COV-1), and Middle East respiratory syndrome-related coronavirus (MERS-COV): influenza viruses such as H1N1, H5N1; Acinetobacter baumannii, Escherichia coli, MRSA, Haemophilus influenzae, Klebsiella pneumoniae, or Candida albicans. In aspects, the pathogen is a virus, and in aspects, the virus is SARS-COV-2. It is noted that certain cytokines, such as IL-6, have been shown to be elevated in the plasma of patients with exacerbated COVID-19 pathology. Non-structural protein 10 (NSP10) of SARS-COV-2 has been implicated in the induction of IL-6 and can stimulate IL-6 production from tissue cells, such as lung cancer cell lines, breast cancer cells and the like. Surprisingly, pre-treatment of these tissue cells (e.g. lung and breast cancer cell lines) with the proteins of interest produced by the methods described herein, such as, for example, IL-38 and IL-37, resulted in the modulation (e.g. dampening or attenuation) of the induction of cytokine (e.g. IL-6) secretion from the tissue cells (see, Example 12, FIG. 20). In this way, IL-37 and IL-38 produced by the methods descried herein may reduce SARS-COV-2-induced inflammation. These effects may also be dose-dependent. Thus, the proteins of interest produced by the methods described herein may alter the cytokine secretion profile of tissue cells (e.g. lung cells), which may have implications for preventing or attenuating cytokine release syndrome associated with diseases like COVID-19, either as a preventative treatment or as a treatment for an already diagnosed case.

Thus, in aspects, a method of treating an inflammatory disease is provided. The inflammatory disease can be, for example, inflammation accompanying various arthritis (for example, rheumatoid arthritis, osteoarthritis), pneumonia, hepatitis (including viral hepatitis), inflammation accompanying infectious diseases (e.g. COVID-19), allergic disease, cardiovascular disease, atherosclerosis, multiple sclerosis, cancers, inflammatory bowel diseases, intestinal enteritis, nephritis (inflammation accompanying glomerular nephritis, nephrofibrosis), gastritis, angiitis, pancreatitis, peritonitis, bronchitis, myocarditis, cerebritis, inflammation in postischemic reperfusion injury (myocardial ischemic reperfusion injury), inflammation attributed to immune rejection after transplantation of tissue and organ, burn, various skin inflammation (psoriasis, allergic contact-type dermatitis, lichen planus which is chronic inflammatory skin disease), inflammation in multiple organ failure, inflammation after operation of percutaneous transluminal coronary angioplasty (PTCA), and inflammation accompanying arteriosclerosis, and autoimmune thyroiditis.

In aspects, the method comprises administering the protein of interest produced by the methods described herein, to a patient in need thereof. Thus, the method typically comprises transforming the plastid described herein, with the transformation vector described herein, expressing the protein of interest, purifying the protein of interest and administering the protein of the interest to a patient in need thereof. In aspects, use of the protein of interest for treating the inflammatory disease is provided, wherein the protein of interest is produced from the transformation vector described herein by the methods described herein. The protein of interest may be prepared in a suitable vehicle for medical application to the patient. For example, the purified protein of interest may be prepared as a composition prepared by per se known methods for the preparation of pharmaceutically acceptable compositions that can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, 20th ed., Mack Publishing Company, Easton, Pa., USA, 2000). On this basis, the compositions may include, albeit not exclusively, the protein of interest in association with one or more pharmaceutically acceptable vehicles or diluents, and may be contained in buffered solutions with a suitable pH that are iso-osmotic with physiological fluids.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of the patient. Various excipients such as binders, fillers, lubricants, glidants, disintegrating agents, colorants, pigments, wicking agents, extrusion aids, plasticizers, sustained release agents, anti-static agents, anti-tacking agents, diluents, gelling agents, hydrophilic and hydrophobic polymers, water-soluble and water insoluble polymers, and the like, could be added to the composition, as would be understood, prior to administering the composition to the patient.

The pharmaceutical compositions of the invention can, in aspects, be administered for example, by parenteral, intravenous, subcutaneous, intradermal, intramuscular, intracranial, intraorbital, ophthalmic, intraventricular, intracapsular, intraspinal, intracisternal, intraperitoneal, intranasal, intrarectal, aerosol or oral administration. Typically, the compositions of the invention are administered subcutaneously, intramuscularly, or intradermally. In addition, compositions may administered once or may be repeated several times. For example, the composition may be administered daily, weekly, monthly, yearly, or a combination thereof, depending upon the inflammatory disease state or the level of inflammation that needs to be treated.

Also provided is a protein for use in treating a condition (e.g., an inflammatory disorder), wherein the protein is obtained by the method of the invention. The invention also provides a use of protein of interest produced by the methods described herein for the manufacture of a medicament (e.g. an inflammatory disease). For example, the protein may be obtained by a method comprising the steps of transforming a plastid with the vector of the invention such that the vector integrates into the plastid genome, and growing the plant cell such that the protein is expressed.

Also provided is a pharmaceutical composition for use in treating a condition (e.g., an inflammatory disorder), wherein the pharmaceutical composition is obtained by the method of the invention. For example, the pharmaceutical composition may be obtained by the steps of transforming a plastid with the vector of the invention such that the vector integrates into the plastid genome, growing the plant cell such that a protein of interest is expressed, purifying the protein and formulating the protein with one or more pharmaceutically acceptable excipients.

Also provided is a method of manufacturing a pharmaceutical composition, the method comprising the steps of transforming a plastid with the vector of the invention such that the vector integrates into the plastid genome, growing the plant cell such that a protein of interest is expressed, purifying the protein and formulating the protein with one or more pharmaceutically acceptable excipients.

### Aspects of the Invention

The below aspects describe the invention in general terms and may be combined with any of the disclosure of the invention provided herein.

1. An expression cassette comprising a heterologous polynucleotide sequence encoding a protein of interest, wherein the heterologous polynucleotide sequence is operably linked to a regulatory sequence.

2. The expression cassette of aspect 1, wherein the regulatory sequence is operative in the plastid.

3. A vector comprising the expression cassette of aspects 1 or 2.

4. A vector comprising the expression cassette of aspect 1 or 2, wherein the vector comprises a first and a second flanking sequence that allow for stable integration of the heterologous polynucleotide sequence into a plastid genome, wherein one of the flanking sequences is 5′ to the expression cassette and the other flanking sequence is 3′ to the expression cassette.

5. The vector of any one of aspects 3 or 4 wherein the vector is a transformation vector.

6. The vector of aspect 5 for stably transforming a plastid.

7. A transformation vector for stably transforming a plastid, comprising, an expression cassette, comprising, as operably-linked components, a regulatory sequence operative in the plastid, a heterologous polynucleotide sequence coding for a protein of interest, and, flanking each side of the expression cassette, a first DNA flanking sequence and a second flanking DNA sequence which allow for stable integration of the heterologous polynucleotide sequence coding for the protein of interest into the plastid genome.

8. The expression cassette or vector according to any one of the preceding aspects, comprising in the 5′ to 3′ direction: a first flanking sequence (e.g., a trnl sequence), a spacer (e.g., a psbN-psbH spacer), a promoter sequence (e.g., a core promoter such as a PpsbA core promoter), a 5′UTR (e.g., a psbA 5′UTR), a heterologous polynucleotide encoding a protein of interest (e.g., IL-37b) and a 3′ UTR (e.g., a TrbcL 3′UTR).

9. The expression cassette or vector according to any one of the preceding aspects, further comprising a Shine Dalgarno sequence; optionally wherein the Shine Dalgarno sequence is 5′ to the promoter sequence; further optionally wherein the Shine Dalgarno sequence is immediately 5′ to the promoter sequence.

10. The expression cassette or vector according to any one of the preceding aspects, further comprising polynucleotide sequence encoding a selection marker; optionally wherein the selection marker is an antibiotic resistance gene; further optionally wherein the marker is a spectinomycin resistance gene (e.g., SEQ ID NO: 37).

11. The vector of any one of aspects 3 to 10, wherein the first and second flanking are substantially homologous to sequences at or around an integration site of the plastid genome.

12. The vector of any one of aspects 3 to 11 wherein the flanking sequences have at least 80% sequence identity, at least 90% sequence identity, at least 95% sequence identity, at least 98% sequence identity to a plastid genome sequences.

13. The vector of any one of aspects 3 to 12, wherein the flanking sequences comprise or consist of a sequence of from 500 to 3000 base pairs, from 750 to 3000 base pairs, from 1000 to 2750 base pairs, from 1250 to 2500 base pairs, or from 1500 to 2250 base pairs.

14. The vector of any one of aspects 3 to 13, wherein the flanking sequences comprise or consist of a plastid sequence of from 500 to 3000 base pairs, from 750 to 3000 base pairs, from 1000 to 2750 base pairs, from 1250 to 2500 base pairs, or from 1500 to 2250 base pairs.

15. The vector of any one of aspects 3 to 14, wherein the flanking sequences comprise or consist of a sequence from the tobacco plastome (NC_001879), tomato plastome (NC_007898) or the Chrysanthemum indicum plastome (NC_020320).

16. The vector of any one of aspects 3 to 15, wherein the first flanking sequence comprises trnl, rps12 or trnT sequence.

17. The vector of any one of aspects 3 to 16, wherein the second flanking sequence comprise trnA, or tmnV or trnG 18. The vector of any one of aspects 3 to 17, wherein the vector comprises a pair of flanking sequences selected from: a trnl sequence and a trnA sequence; a trnl sequence and a sequence gene; a trnl sequence and a trnG sequence; a rps 12 sequence and a trnA sequence; a rps12 sequence and a trnV sequence; a rps12 sequence and a trnG sequence; a trnT sequence and a trnA sequence; a trnT sequence and a trnV sequence; and a trnT sequence and a trnG sequence.

19. The vector of any one of aspects 3 to 18 wherein the first and second flanking sequences are engineered such that, following transformation, the heterologous polynucleotide encoding the protein of interest is inserted in the intergenic region between either: (i) the trnl and trnA genes of the rrn16 operon, (ii) the rps 12 and trnV genes of the tobacco plastome (NC_001879) or tomato plastome (NC_007898), or (iii) the tmT and trnG genes of the Chrysanthemum indicum plastome (NC_020320).

20. The expression cassette or vector of any one of the preceding aspects, further comprising a spacer sequence; optionally wherein the spacer sequence is an intergenic plastid sequence.

21. The expression cassette or vector of any one of the preceding aspects, wherein the spacer sequence comprises (or consists of) a plastid genome sequence (e.g., from a higher plant), wherein the sequence is from 55 to 80 base pairs, from 60 to 80 base pairs, from 65 to 80 base pairs, from 70 to 80 base pairs or from 75 to 80 base pairs long.

22. The expression cassette or vector of any one of the preceding aspects, wherein the spacer sequence comprises (or consists of) the sequences between the psbN and psbH genes, between the rps2 and atpl genes, or between the rpoC2 and rps2 genes in a plastid genome.

23. The expression cassette or vector of any one of the preceding aspects, wherein the spacer comprise (or consist of) a sequence having at least 70% sequence identity, at least 80% sequence identity, at least 90% sequence identity or at least 95% sequence identity to an intergenic sequence between the psbN and psbH genes, the rps2 and atpl genes, or the rpoC2 and rps2 genes.

24. The expression cassette or vector of any one of the preceding aspects, wherein the spacer comprises (or consists of) a polynucleotide sequence having at least 70% sequence identity, at least 80% sequence identity, at least 90% sequence identity or at least 95% sequence identity to one of SEQ ID NO: 23 to SEQ ID NO: 26.

25. The expression cassette or vector of any one of the preceding aspects, wherein the spacer is 3′ to one of the flanking sequences; optionally wherein the spacer is immediately 3′ to the flanking sequence.

26. The expression cassette or vector of any one of the preceding aspects, wherein the spacer is 5′ to the heterologous polynucleotide encoding the protein of interest.

27. The expression cassette or vector of any one of the preceding aspects, wherein the spacer comprises (or consists of) a sequence of from 50 to 80 base pairs, wherein the sequence has at least 80% homology to an intergenic plastid sequence from between psbN and psbH genes, rps2 and atpl genes or rpoC2 and rps2 genes.

28. The expression cassette or vector of any one of the preceding aspects, wherein the regulatory sequence comprises (or consists of) a promoter that is operative in a plastid.

29. The expression cassette or vector of any one of the preceding aspects, wherein the heterologous polynucleotide sequence is operably linked to a promoter.

30. The expression cassette or vector of any one of the preceding aspects, comprising a 16S rRNA promoter, a psbA promoter or a rbcL promoter.

31. The expression cassette or vector of any one of the preceding aspects, wherein the promoter is a truncated promoter (e.g., a core promoter); optionally wherein the truncated promoter substantially retains the activity of the full length promoter.

32. The expression cassette or vector of any one of the preceding aspects, comprising a modified promoter optionally wherein the modified promoter is a modified 16S rRNA promoter, a modified psbA promoter or a modified rbcL promoter.

33. The expression cassette or vector of any one of the preceding aspects, wherein the promoter has at least 80% sequence homology to a 16S rRNA promoter, a psbA promoter or a rbcL promoter; optionally wherein the modified promoter has substantially equal functionality to that of the endogenous promoter.

34. The expression cassette or vector of any one of the preceding aspects, wherein the promoter comprises a sequence selected from SEQ ID NO: 34 to 36.

35. The expression cassette or vector of any one of the preceding aspects, further comprising a 5′ untranslated region (UTR).

36. The expression cassette or vector of aspect 35, wherein the 5′ UTR enhances transcription and/or translation of the gene of interest/protein of interest.

37. The expression cassette or vector of aspect 35 or 36, wherein the 5′ UTR is selected from a psbA 5′UTR, a rbcL 5′UTR and the bacteriophage T7gene 10 (T7G10) 5′UTR.

38. The expression cassette or vector of any one of aspects 35 to 37, wherein the 5′ UTR is a 5′ UTR of psbA or a 5′ UTR of T7G10.

39. The expression cassette or vector of any one of aspects 35 to 38, wherein the 5′ UTR is a 5′ UTR of psbA.

40. The expression cassette or vector of any one of aspects 35 to 39, wherein the 5′ UTR is a 5′ UTR of T7G10.

41. The expression cassette or vector of any one of aspects 35 to 40, wherein the 5′ UTR is selected from SEQ ID NO. 27, SEQ ID NO: 28 or SEQ ID NO:29.

42. The expression cassette or vector of any one of the preceding aspects, further comprising a 3′ untranslated region (UTR).

43. The expression cassette or vector of aspect 42, wherein the 3′UTR enhances the stability of a transcript encoding the protein of interest.

44. The expression cassette or vector of aspect 42 or 43, wherein the 3′ UTR is a psbA 3′ UTR, a psbC 3′ UTR or a rbcL 3′ UTR.

45. The expression cassette or vector of aspect 44, wherein the 3′ UTR is a 3′ UTR of psbA.

46. The expression cassette or vector of aspect 44, wherein the 3′ UTR is a 3′ UTR of a (heterologous) psbC gene.

47. The expression cassette or vector of aspect 44, wherein the 3′ UTR is a 3′ UTR of a rbcL gene.

48. The expression cassette or vector of any one of aspects 42 to 47, wherein the 3′ UTR comprises a sequence selected from SEQ ID NO: 30 to 33.

49. The expression cassette or vector of any one of the preceding aspects, further comprising a DNA sequence coding for a selectable marker; optionally wherein the selectable marker is an antibiotic resistant selectable marker; further optionally wherein the antibiotic resistant selectable marker is aadA (e.g., SEQ ID NO: 37).

50. The expression cassette or vector of any one of the preceding aspects, wherein the heterologous polynucleotide sequence is codon-optimized.

51. The expression cassette or vector of any one of the preceding aspects, wherein sequence coding for the protein of interest comprises a purification tag; optionally a c-myc tag (SEQ ID NO. 1) or a histidine tag such as 7×HIS tag (SEQ ID NO. 2).

52. The expression cassette or vector of any one of the preceding aspects, wherein the plastid is selected from a chloroplast, a chromoplast, an amyloplast, a proplastid, a leucoplast or an etioplast; preferably wherein the plastid is a chloroplast.

53. The expression cassette or vector of any one of the preceding aspects, wherein the plastid is from a monocot or dicot plant; optionally wherein the dicot plant is a low-nicotine tobacco plant.

54. The expression cassette or vector of any one of the preceding aspects, wherein the plastid is a chloroplast, a chromoplast, an amyloplast, a proplastid, a leucoplast or an etioplast from a monocot plant; optionally wherein the plastid is a chloroplast from a monocot plant.

55. The expression cassette or vector of any one of the preceding aspects, wherein the plastid is a chloroplast, a chromoplast, an amyloplast, a proplastid, a leucoplast or an etioplast from a dicot plant; optionally wherein the plastid is a chloroplast from a dicot plant; further optionally wherein the plastid is a chloroplast from a low-nicotine tobacco plant.

56. The expression cassette or vector of any one of the preceding aspects, wherein the protein of interest is a therapeutic protein.

57. The expression cassette or vector of any one of the preceding aspects, wherein the protein of interest is a human protein.

58. The expression cassette or vector of any one of the preceding aspects, wherein the protein of interest is suitable for administration to a human subject; further optionally wherein the administration occurs after purification.

59. The expression cassette or vector of any one of the preceding aspects, wherein the protein of interest is a monomer.

60. The expression cassette or vector of any one of the preceding aspects, wherein the protein of interest assembles into a multimer (e.g., a dimer or a trimer) when expressed in a plastid.

61. The expression cassette or vector of any one of the preceding aspects, wherein the protein of interest assembles into a multimer (e.g., a dimer or a trimer) when expressed in a plastid but is substantially monomeric when expressed in bacterial expression systems.

62. The expression cassette or vector of any one of the preceding aspects, wherein the protein of interest is a cytokine; optionally
wherein
(i) the cytokine is a human cytokine; and/or
(ii) the cytokine is selected from: IL-38, IL-38b, IL-37, IL-37b, IL-33, G-CSF, IL-11, IL-33, IL-1Ra, IL-36Ra, IL-2, IL-3, IL-10, CSF3, IL-13, FGF19, CSF23, IL-35, leukemia inhibitory factor (LIF), IL-6, IL-4, BMP2, BMP7, TGF-β1 and Staphylococcus aureus Protein A. 63. The expression cassette or vector of any one of the preceding aspects, wherein the cytokine is selected from IL-38 (e.g., human IL-38), IL-37b (e.g., human IL-37b), IL-33 (e.g., human IL-33) and CSF3 (e.g., human CSF3).

64. The expression cassette or vector of any one of the preceding aspects, wherein the protein of interest is encoded by a polynucleotide having at least 70% sequence identity (e.g., at least 80% sequence identity or 90% sequence identity) to SEQ ID NOS: 3 to 22; optionally wherein the protein of interest is encoded by a polynucleotide having at least 70% sequence identity (e.g., at least 80% sequence identity or 90% sequence identity) to SEQ ID NO. 3 or SEQ ID NO. 5.

65. The expression cassette or vector of any one of the preceding aspects, wherein the protein of interest is encoded by a polynucleotide having the sequence defined any one of SEQ ID NOs: 3 to 22; optionally wherein the protein of interest is encoded by SEQ ID NO. 3 or SEQ ID NO. 5.

66. The expression cassette or vector according to any one of the preceding aspects, wherein following expression of the heterologous polynucleotide in a plastid, the protein of interest is present in an amount of about 0.1% to about 60% of total soluble protein (TSP) (e.g., from 0.1% to 60% TSP); optionally wherein the protein of interest is present in an amount of from 0.1% to 60%, from 0.5% to 60%, from 1% to 60%, from 5% to 60%, from 10% to 60%, from 15% to 60%, from 20% to 60%, from 25% to 60%, from 30% to 60%, from 35% to 60%, from 40% to 60%, from 45% to 60%, from 50% to 60% or from 55% to 60% TSP.

67. The expression cassette or vector according to any one of the preceding aspects, wherein the protein of interest is expressed in an amount of about 0.1% to about 60% of total soluble protein (TSP) (e.g., from 0.1% to 60% TSP); optionally wherein the protein of interest is expressed in an amount of from 0.1% to 60%, from 0.5% to 60%, from 1% to 60%, from 5% to 60%, from 10% to 60%, from 15% to 60%, from 20% to 60%, from 25% to 60%, from 30% to 60%, from 35% to 60%, from 40% to 60%, from 45% to 60%, from 50% to 60% or from 55% to 60% TSP.

68. The expression cassette or vector according to any one of the preceding aspects, wherein following expression of the heterologous polynucleotide in a plastid, the protein of interest is present as a monomer or multimer (e.g., a dimer or trimer) in an amount of from 0.1% to 60%, from 0.5% to 60%, from 1% to 60%, from 5% to 60%, from 10% to 60%, from 15% to 60%, from 20% to 60%, from 25% to 60%, from 30% to 60%, from 35% to 60%, from 40% to 60%, from 45% to 60%, from 50% to 60% or from 55% to 60% TSP.

69. The expression cassette or vector according to any one of the preceding aspects, further comprising one or more of:
(a) a sequence homologous to an intergenic spacer, optionally wherein the sequence is selected from SEQ ID NO: 23 to 26;
(b) a 5′ UTR, optionally wherein the 5′ UTR is selected from SEQ ID NO: 27 to SEQ ID NO: 29;
(c) a 3′ UTR, optionally wherein the 5′ UTR is selected from SEQ ID NO: 30 to 33;
(d) a promoter sequence; optionally wherein the promoter sequence is operable in a plastid (e.g. a plastid of a higher plant); further optionally wherein the promoter is a core promoter; preferably wherein the core promoter is selected from SEQ ID NO: 34 and 35;
(e) a Shine-Dalgarno sequence; and/or
(f) a sequence encoding a selection marker; optionally wherein the sequence encodes aminoglycoside acetyltransferase marker and/or wherein expression of the marker enables selection by spectinomycin resistance; further optionally wherein the selection marker is encoded by a sequence comprising or consisting of SEQ ID NO: 37.

70. A vector comprising an expression cassette, the expression cassette comprising a heterologous polynucleotide sequence encoding IL-38 (e.g., human IL-38), wherein the heterologous polynucleotide sequence is operably linked to a regulatory sequence comprising or consisting of a psbA promoter sequence (e.g., a core promoter sequence), and wherein the vector comprises a first and a second flanking sequence that allow for stable integration of the expression cassette into the plastid genome, wherein one of the flanking sequences is 5′ to the expression cassette and the other flanking sequence is 3′ to the expression cassette; optionally
wherein
the expression cassette further comprises:
   a spacer region comprising a sequence from between psbN and psbH genes;
   a 5′UTR comprising T7G10;
   a 3′ UTR comprising psbC; and/or
   a selectable marker comprising aadA.

71. A vector comprising an expression cassette, the expression cassette comprising a heterologous polynucleotide sequence encoding protein A of Staph aureus, wherein the heterologous polynucleotide sequence is operably linked to a regulatory sequence comprising or consisting of a psbA promoter sequence, and wherein the vector comprises a first and a second flanking sequence that allow for stable integration of the expression cassette into the plastid genome; wherein one of the flanking sequences is 5′ to the expression cassette and the other flanking sequence is 3′ to the expression cassette; optionally
wherein
the expression cassette further comprises:
   a spacer region comprising a sequence from between psbN and psbH genes;
   a 5′UTR comprising T7G10;
   a 3′ UTR comprising psbC; and/or
   a selectable marker comprising aadA.

72. A vector comprising an expression cassette, the expression cassette comprising a heterologous polynucleotide sequence encoding IL-37b (human IL-37b) wherein the heterologous polynucleotide sequence is operably linked to a regulatory sequence comprising or consisting of a psbA promoter sequence, and wherein the vector comprises a first and a second flanking sequence that allow for stable integration of the expression cassette into the plastid genome; wherein one of the flanking sequences is 5′ to the expression cassette and the other flanking sequence is 3′ to the expression cassette; optionally
wherein
the first DNA flanking sequence comprises trnl and the second flanking DNA sequence comprises trnA.

73. A vector comprising an expression cassette, the expression cassette comprising a heterologous polynucleotide sequence encoding IL-33 (human IL-33) wherein the heterologous polynucleotide sequence is operably linked to a regulatory sequence comprising or consisting of a psbA promoter sequence, and wherein the vector comprises a first and a second flanking sequence that allow for stable integration of the expression cassette into the plastid genome; wherein one of the flanking sequences is 5′ to the expression cassette and the other flanking sequence is 3′ to the expression cassette; optionally
wherein
the first DNA flanking sequence comprises trl and the second flanking DNA sequence comprises trnA.

74. A vector comprising an expression cassette, the expression cassette comprising a heterologous polynucleotide sequence encoding G-CSF (human G-CSF) wherein the heterologous polynucleotide sequence is operably linked to a regulatory sequence comprising or consisting of a psbA promoter sequence, and wherein the vector comprises a first and a second flanking sequence that allow for stable integration of the expression cassette into the plastid genome; wherein one of the flanking sequences is 5′ to the expression cassette and the other flanking sequence is 3′ to the expression cassette; optionally
wherein
the first DNA flanking sequence comprises trl and the second flanking DNA sequence comprises trnA.

75. The vector of any one of aspects 72 to 74, wherein the expression cassette further comprises a spacer region between psbN and psbH genes.

76. The vector of any one of aspects 72 to 75, wherein the expression cassette further comprises a 5′UTR comprising psbA.

77. The vector of any one of any one of aspects 72 to 76, wherein the expression cassette further comprises a 3′ UTR comprising psbC.

78. The vector of any one of aspects 72 to 77, wherein the expression cassette further comprises a selectable marker comprising aadA.

79. A plant plastid, plant cell or plant that has been stably transformed with the expression cassette or vector of any one of the preceding aspects.

80. The plant of aspect 79, wherein mature leaves, young leaves or old leaves are transformed with the expression cassette or vector.

81. The plant of aspect 79 or 80, wherein mature leaves, young leaves or old leaves express the protein of interest.

82. The plant of any one of aspects 79 to 81, wherein the protein of interest is present in the plastid in an amount of from 0.1% to 60%, from 0.5% to 60%, from 1% to 60%, from 5% to 60%, from 10% to 60%, from 15% to 60%, from 20% to 60%, from 25% to 60%, from 30% to 60%, from 35% to 60%, from 40% to 60%, from 45% to 60%, from 50% to 60% or from 55% to 60% TSP.

83. The plant of any one of aspects 79 to 83, wherein the protein of interest is present in the plastid as a monomer or assembled as a multimer (e.g. a dimer or trimer) and wherein said monomer or multimer is present in the plastid in an amount of from 0.1% to 60%, from 0.5% to 60%, from 1% to 60%, from 5% to 60%, from 10% to 60%, from 15% to 60%, from 20% to 60%, from 25% to 60%, from 30% to 60%, from 35% to 60%, from 40% to 60%, from 45% to 60%, from 50% to 60% or from 55% to 60% TSP.

84. A progeny of the plant of any one of aspects 79 to 83.

85. A seed of the plant of any one of aspects 79 to 84.

86. A method for producing a protein of interest, the method comprising: integrating the vector of any one of aspects 3 to 78 into a plastid genome of a plant cell; and growing the plant cell to thereby express the protein of interest.

87. A method for producing a protein of interest, the method comprising transforming a plastid with the vector of any one of aspects 3 to 78 such that the vector integrates into the plastid genome; and expressing the protein of interest.

88. A method for manufacturing a pharmaceutical composition, the method comprising transforming a plastid with the vector of any one of aspects 3 to 78 such that the vector integrates into the plastid genome; expressing the protein of interest; and formulating with one or more pharmaceutically acceptable excipients.

89. The method of any one of aspects 86 to 88, wherein the vector is integrated into the plastid genome by homologous recombination.

90. The method of any one of aspects 86 to 89, wherein the vector is stably integrated into the plastid genome.

91. The method of any one of aspects 86 to 90, wherein the plastid genome is from a low-alkaloid tobacco plant.

92. The method of any one of aspects 86 to 91, further comprising recovering the protein of interest.

93. The method of aspect 92, wherein the recovering comprises isolating and purifying the protein of interest.

94. The method of aspect 93, wherein the purifying comprising using an IMAC procedure.

95. The method of any one of aspects 96 to 94, wherein the protein of interest is an immune modulator; optionally wherein the protein is able to modulate an immune response ex vivo or in vitro.

96. The method of aspect 95, wherein the protein of interest dose-dependently modulates the immune response.

97. The method of aspect 95 or 96, wherein the immune response comprises modulation of peripheral blood mononuclear cells cytokine secretion in response to inflammatory mediator stimulation, optionally wherein the inflammatory mediator comprises LPS or PHA.

98. The method of any one of aspects 95 to 97, wherein the immunogenic response comprises modulation of tissue cell cytokine secretion in response to viral stimulation; optionally wherein the viral stimulation is SARS-COV-2 stimulation and/or the tissue cell is a lung cell.

99. Use of the vector of any one of aspects 3 to 78 for stably transforming a plant cell or plant.

100. Use of the transformation vector of any one of aspects 3 to 78 for producing a protein of interest.

101. The use of aspect 100, wherein the protein modulates an immune response; optionally wherein the immune response comprises cytokine secretion from peripheral blood mononuclear cells; and/or cytokine secretion from tissue cells in response to SARS-COV-2 stimulation.

102. A method of treating an inflammatory disorder comprising administering the protein of interest produced from the transformation vector of any one of aspects 3 to 78 to a patient in need thereof.

103. Use of the protein of interest produced from the transformation vector of any one of aspects 3 to 78 for treating an inflammatory disorder in a patient in need thereof.

104. A composition comprising at least one excipient and the protein of interest produced from the transformation vector of any one of aspects 3 to 78.

105. Use of the composition of aspect 104 for treating an inflammatory disorder.

106. A protein of interest produced from the transformation vector of any one of aspects 3 to 78.

107. A protein of interest produced by the method of any one of aspects 86 to 98.

108. Use of the protein of interest of aspect 106 or 107, for treating an inflammatory disorder.

109. Use of the expression cassette or vector according to any one of aspects 1 to 78 for the manufacture of a medicament; optionally wherein the medicament is for treating an inflammatory disorder.

110. A protein for use in a method of treating a disorder (e.g., an inflammatory disorder), wherein the protein is obtained by the method of any one of aspects 86 to 98.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example 1. Synthesis and Sequence of Gene of Interest (GOI) Proteins

All the genes of interest (GOIs) for the proteins of interest described herein were synthesized as codon-optimized DNA sequences according to Nakamura, M. and Sugiura, M. (2007) Translation efficiencies of synonymous codons are not always correlated with codon usage in tobacco chloroplasts. Plant J. 49, 128-134 (the entirety of which is hereby incorporated by reference), with modifications (Table I). Open reading frame of the GOIs translationally fused to the DNA sequence fragment encoding the 10 amino acids of the c-myc tag (EQKLISEEDL) (SEQ ID NO. 1) or the 7×HIS tag (HHHHHHH) (SEQ ID NO. 2) with a stop codon ‘TAA’.

TABLE I
Synonymous codons preferences (I - first, II - second, III
- third-preferable) used for optimization of the synthetic
genes encoding the cytokines under study. All the GOI reading
frames were synthesized using first-preference codons; in
some cases second and third-preferable codons were used to
omit formation of unwanted sites of restriction nucleases.

| | Codon Preference (I > II > III) | | |
|---|---|---|---|
| Amino Acid | I | II | III |
| G | GGA | | |
| D | GAT | | |
| N | AAT | | |
| K | AAA | | |
| Q | CAA | | |
| Y | TAC | | |
| F | TTC | | |
| E | GAA | | |
| S | TCT | AGT | TCA |
| L | TTA | CTT | |
| P | CCT | CCA | |
| R | AGA | CGA | CGT |
| I | ATT | ATA | |
| T | ACT | ACA | |
| C | TGT | | |
| H | CAT | | |
| V | GTA | GTT | |
| W | TGG | | |
| A | GCT | GCC | GCA |
| M | ATG | | |

The DNA sequences of the synthesized gene sequences were:

GOI#1 rhIL-38
(SEQ ID NO. 3)
ATGTCTTGTTCTTTACCTATGGCTAGATACTACATTATTAAATACGCTGATCAAAAAGCCTTA
TACACTAGAGATGGACAATTATTAGTAGGAGATCCTGTAGCTGATAATTGTTGTGCTGAAAA
AATTTGTATTTTACCTAATAGAGGATTAGCTAGAACTAAAGTACCTATTTTCTTAGGAATTCA
AGGAGGATCTAGATGTTTAGCTTGTGTAGAAACTGAAGAAGGACCTTCTTTACAATTAGAAG
ATGTAAATATTGAAGAATTATACAAAGGAGGAGAAGAAGCTACTAGATTCACTTTCTTCCAA
TCTTCTTCTGGATCTGCTTTCAGATTAGAAGCTGCTGCTTGGCCTGGATGGTTCTTATGTGG
ACCAGCTGAACCTCAACAACCTGTACAATTAACTAAAGAATCTGAACCTTCTGCTAGAACTA
AATTCTACTTCGAACAATCTTGG
GOI#2 rhIL-11
(SEQ ID NO. 4)
ATGCCTGGACCTCCTCCTGGACCTCCTAGAGTATCTCCTGATCCTAGAGCTGAATTAGATT
CTACTGTATTATTAACTAGATCTTTATTAGCTGATACTAGACAATTAGCTGCTCAATTAAGAG
ATAAATTCCCTGCTGATGGAGATCATAATTTAGATTCTTTACCTACTTTAGCTATGTCTGCTG
GAGCTTTAGGAGCTTTACAATTACCTGGAGTATTAACTAGATTAAGAGCTGATTTATTATCTT
ACTTAAGACATGTACAATGGTTAAGAAGAGCTGGAGGATCTTCTTTAAAAACTTTAGAACCT
GAATTAGGAACTTTACAAGCTAGATTAGATAGATTATTAAGAAGATTACAATTATTAATGTCT
AGATTAGCTTTACCTCAACCTCCTCCTGATCCTCCTGCTCCTCCTTTAGCTCCTCCTTCTTC
TGCTTGGGGAGGAATTAGAGCTGCTCATGCTATTTTAGGAGGATTACATTTAACTTTAGATT
GGGCTGTAAGAGGATTATTATTATTAAAAACTAGATTA
GOI#3rhIL-37b
(SEQ ID NO. 5)
ATGGTACATACTTCTCCTAAAGTAAAAAATTTAAATCCTAAAAAATTCTCTATTCATGATCAA
GATCATAAAGTATTAGTATTAGATTCTGGAAATTTAATTGCTGTACCTGATAAAAATTACATT
AGACCTGAAATTTTCTTCGCTTTAGCTTCTTCTTTATCTTCTGCTTCTGCTGAAAAAGGATCT
CCTATTTTATTAGGAGTATCTAAAGGAGAATTCTGTTTATACTGTGATAAAGATAAAGGACAA
TCTCATCCTTCTTTACAATTAAAAAAAGAAAAATTAATGAAATTAGCTGCTCAAAAAGAATCT
GCTAGAAGACCTTTCATTTTCTACAGAGCTCAAGTAGGATCTTGGAATATGTTAGAATCTGC
TGCTCATCCTGGATGGTTCATTTGTACTTCTTGTAATTGTAATGAACCTGTAGGAGTAACTG
ATAAATTCGAAAATAGAAAACATATTGAATTCTCTTTCCAACCTGTATGTAAAGCTGAAATGT
CTCCTTCTGAAGTATCTGAT
GOI#4 rhIL-33
(SEQ ID NO. 6)
ATGAAACCTAAAATGAAATACTCTACTAATAAAATTTCTACTGCTAAATGGAAAAATACTGCT
TCTAAAGCTTTATGTTTCAAATTAGGAAAATCTCAACAAAAAGCTAAAGAAGTATGTCCTATG
TACTTCATGAAATTAAGATCTGGATTAATGATTAAAAAAGAGGCTTGTTACTTCAGAAGAGA
AACTACTAAAAGACCTTCTTTAAAAACTGGAATTTCTCCTATTACTGAATACTTAGCTTCTTT
ATCTACTTACAATGATCAATCTATTACTTTCGCTTTAGAAGATGAATCTTACGAAATTTACGT
AGAAGATTTAAAAAAAGATGAAAAAAAAGATAAAGTATTATTATCTTACTACGAATCTCAACA
TCCTTCTAATGAATCTGGAGATGGAGTAGATGGAAAAATGTTAATGGTAACTTTATCTCCTA
CTAAAGATTTCTGGTTACATGCTAATAATAAAGAACATTCTGTAGAATTACATAAATGTGAAA
AACCTTTACCTGATCAAGCATTCTTCGTATTACATAATATGCATTCTAATTGTGTATCTTTCG
AATGTAAAACTGATCCTGGAGTATTCATTGGAGTAAAAGATAATCATTTAGCTTTAATTAAAG
TAGATTCTTCTGAAAATTTATGTACTGAAAATATTTTATTCAAATTATCTGAAACT
GOI#5 rhIL-1Ra
(SEQ ID NO. 7)
ATGAGACCTTCTGGAAGAAAATCTTCTAAAATGCAAGCATTCAGAATTTGGGATGTAAATCA
AAAAACTTTCTACTTAAGAAATAATCAATTAGTAGCTGGATACTTACAAGGACCTAATGTAAA
TTTAGAAGAAAAAATTGATGTAGTACCTATTGAACCTCATGCTTTATTCTTAGGAATTCATGG
AGGAAAAATGTGTTTATCTTGTGTAAAATCTGGAGATGAAACTAGATTACAATTAGAAGCTG
TAAATATTACTGATTTATCTGAAAATAGAAAACAAGATAAAAGATTCGCTTTCATTAGATCTG
ATTCTGGACCTACTACTTCTTTCGAATCTGCTGCTTGTCCTGGATGGTTCTTATGTACTGCT
ATGGAAGCTGATCAACCTGTATCTTTAACTAATATGCCTGATGAAGGAGTAATGGTAACTAA
ATTCTACTTCCAAGAAGATGAA
GOI#6 rhIL-36Ra
(SEQ ID NO. 8)
ATGGTATTATCTGGAGCTTTATGTTTCAGAATGAAAGATTCTGCTTTAAAAGTATTATACTTA
CATAATAATCAATTATTAGCTGGAGGATTACATGAAGGAAAAGTAATTAAAGGAGAAGAAAT
TTCTGTAGTACCTAATAGATGGTTAGATGCTTCTTTATCTCCTGTAATTTTAGGAGTACAAG
GAGGATCTCAATGTTTATCTTGTGGAGTAGGACAAGAACCTACTTTAACTTTAGAACCTGTA
AATATTATGGAATTATACTTAGTAGCTAAAGAATCTAAATCTTTCACTTTCTACAGAAGAGAT
ATGGGATTAACTTCTTCTTTCGGATCTGCTGCTTACCCTGGATGGTTCTTATGTACTGTACC
TGAAGCTGATCAACCTGTAAGATTAACTCAATTACCTGAAAATGGAGGATGGAATGCTCCTA
TTACTGATTTCTACTTCCAACAATGTGAT
GOI#7 rhIL-2
(SEQ ID NO. 9)
ATGGCTCCTACTTCTTCTTCTACTAAAAAAACTCAATTACAATTAGAACATTTATTATTAGATT
TACAAATGATTTTAAATGGAATTAATAATTACAAAAATCCTAAATTAACTAGAATGTTAACTTT
CAAATTCTACATGCCTAAAAAAGCTACTGAATTAAAACATTTACAATGTTTAGAAGAAGAATT
AAAACCTTTAGAAGAAGTATTAAATTTAGCTCAATCTAAAAATTTCCATTTAAGACCTAGAGA
TTTAATTTCTAATATTAATGTAATTGTATTAGAATTAAAAGGATCTGAAACTACTTTCATGTGT
GAATACGCTGATGAAACTGCTACTATTGTAGAATTCTTAAATAGATGGATTACTTTCTGTCAA
TCTATTATTTCTACTTTAACT
GOI#8 rhIL-3
(SEQ ID NO. 10)
ATGGCTCCTATGACTCAAACTACTCCTTTAAAAACTTCTTGGGTAAATTGTTCTAATATGATT
GATGAAATTATTACTCATTTAAAACAACCTCCTTTACCTTTATTAGATTTCAATAATTTAAATG
GAGAAGATCAAGATATTTTAATGGAAAATAATTTAAGAAGACCTAATTTAGAAGCATTCAATA
GAGCTGTAAAATCTTTACAAAATGCTTCTGCTATTGAATCTATTTTAAAAAATTTATTACCTTG
TTTACCTTTAGCTACTGCTGCTCCTACTAGACATCCTATTCATATTAAAGATGGAGATTGGA
ATGAATTCAGAAGAAAATTAACTTTCTACTTAAAAACTTTAGAAAATGCTCAAGCTCAACAAA
CTACTTTATCTTTAGCTATTTTC
GOI#9 rhIL-10
(SEQ ID NO. 11)
ATGGTATTATCTGGAGCTTTATGTTTCAGAATGAAAGATTCTGCTTTAAAAGTATTATACTTA
CATAATAATCAATTATTAGCTGGAGGATTACATGAAGGAAAAGTAATTAAAGGAGAAGAAAT
TTCTGTAGTACCTAATAGATGGTTAGATGCTTCTTTATCTCCTGTAATTTTAGGAGTACAAG
GAGGATCTCAATGTTTATCTTGTGGAGTAGGACAAGAACCTACTTTAACTTTAGAACCTGTA
AATATTATGGAATTATACTTAGTAGCTAAAGAATCTAAATCTTTCACTTTCTACAGAAGAGAT
ATGGGATTAACTTCTTCTTTCGGATCTGCTGCTTACCCTGGATGGTTCTTATGTACTGTACC
TGAAGCTGATCAACCTGTAAGATTAACTCAATTACCTGAAAATGGAGGATGGAATGCTCCTA
TTACTGATTTCTACTTCCAACAATGTGAT
GOI#10 rhCSF3
(SEQ ID NO. 12)
ATGGCTACTCCTTTAGGACCAGCTTCTTCTTTACCTCAATCTTTCTTATTAAAATGTTTAGAA
CAAGTAAGAAAAATTCAAGGAGATGGAGCTGCTTTACAAGAAAAATTAGTATCTGAATGTGC
TACTTACAAATTATGTCATCCTGAAGAATTAGTATTATTAGGACATTCTTTAGGAATTCCTTG
GGCTCCTTTATCTTCTTGTCCTTCTCAAGCATTACAATTAGCTGGATGTTTATCTCAATTACA
TTCTGGATTATTCTTATACCAAGGATTATTACAAGCCTTAGAAGGAATTTCTCCTGAATTAGG
ACCTACTTTAGATACTTTACAATTAGATGTAGCTGATTTCGCTACTACTATTTGGCAACAAAT
GGAAGAATTAGGAATGGCTCCTGCTTTACAACCTACTCAAGGAGCTATGCCTGCTTTCGCT
TCTGCTTTCCAAAGAAGAGCTGGAGGAGTATTAGTAGCTTCTCATTTACAATCTTTCTTAGA
AGTATCTTACAGAGTATTAAGACATTTAGCTCAACCT
GOI#11 rhIL-13
(SEQ ID NO. 13)
ATGGGACCTGTACCTCCTTCTACTGCTTTAAGAGAATTAATTGAAGAATTAGTAAATATTACT
CAAAATCAAAAAGCTCCTTTATGTAATGGATCTATGGTATGGTCTATTAATTTAACTGCTGGA
ATGTACTGTGCTGCTTTAGAATCTTTAATTAATGTATCTGGATGTTCTGCTATTGAAAAAACT
CAAAGAATGTTATCTGGATTCTGTCCTCATAAAGTATCTGCTGGACAATTCTCTTCTTTACAT
GTAAGAGATACTAAAATTGAAGTAGCTCAATTCGTAAAAGATTTATTATTACATTTAAAAAAA
TTATTCAGAGAAGGAAGATTCAAT
GOI#12 rhFGF19
(SEQ ID NO. 14)
ATGTTAGCTTTCTCTGATGCTGGACCTCATGTACATTACGGATGGGGAGATCCTATTAGATT
AAGACATTTATACACTTCTGGACCTCATGGATTATCTTCTTGTTTCTTAAGAATTAGAGCTGA
TGGAGTAGTAGATTGTGCTAGAGGACAATCTGCTCATTCTTTATTAGAAATTAAAGCTGTAG
CTTTAAGAACTGTAGCTATTAAAGGAGTACATTCTGTAAGATACTTATGTATGGGAGCTGAT
GGAAAAATGCAAGGATTATTACAATACTCTGAAGAAGATTGTGCTTTCGAAGAAGAAATTAG
ACCTGATGGATACAATGTATACAGATCTGAAAAACATAGATTACCTGTATCTTTATCTTCTGC
TAAACAAAGACAATTATACAAAAATAGAGGATTCTTACCTTTATCTCATTTCTTACCTATGTT
ACCTATGGTACCTGAAGAACCTGAAGATTTAAGAGGACATTTAGAATCTGATATGTTCTCTT
CTCCTTTAGAAACTGATTCTATGGATCCTTTCGGATTAGTAACTGGATTAGAAGCTGTAAGA
TCTCCTTCTTTCGAAAAA
GOI#13 rhCSF2
(SEQ ID NO. 15)
ATGGCTCCTGCTAGATCTCCTTCTCCTTCTACTCAACCTTGGGAACATGTAAATGCTATTCA
AGAAGCTAGAAGATTATTAAATTTATCTAGAGATACTGCTGCTGAAATGAATGAAACTGTAG
AAGTAATTTCTGAAATGTTCGATTTACAAGAACCTACTTGTTTACAAACTAGATTAGAATTAT
ACAAACAAGGATTAAGAGGATCTTTAACTAAATTAAAAGGACCTTTAACTATGATGGCTTCT
CATTACAAACAACATTGTCCTCCTACTCCTGAAACTTCTTGTGCTACTCAAATTATTACTTTC
GAATCTTTCAAAGAAAATTTAAAAGATTTCTTATTAGTAATTCCTTTCGATTGTTGGGAACCT
GTACAAGAA
GOI#14 rhIL-35
(SEQ ID NO. 16)
ATGAGAAAAGGACCTCCTGCTGCTTTAACTTTACCTAGAGTACAATGTAGAGCTTCTAGATA
CCCTATTGCTGTAGATTGTTCTTGGACTTTACCTCCTGCTCCTAATTCTACTTCTCCTGTATC
TTTCATTGCTACTTACAGATTAGGAATGGCTGCTAGAGGACATTCTTGGCCTTGTTTACAAC
AAACTCCTACTTCTACTTCTTGTACTATTACTGATGTACAATTATTCTCTATGGCTCCTTACG
TATTAAATGTAACTGCTGTACATCCTTGGGGATCTTCTTCTTCTTTCGTACCTTTCATTACTG
AACATATTATTAAACCTGATCCTCCTGAAGGAGTAAGATTATCTCCTTTAGCTGAAAGACAA
TTACAAGTACAATGGGAACCTCCTGGATCTTGGCCTTTCCCTGAAATTTTCTCTTTAAAATA
CTGGATTAGATACAAAAGACAAGGAGCTGCTAGATTCCATAGAGTAGGACCTATTGAAGCT
ACTTCTTTCATTTTAAGAGCTGTAAGACCTAGAGCTAGATACTACGTACAAGTAGCTGCTCA
AGATTTAACTGATTACGGAGAATTATCTGATTGGTCTTTACCAGCTACTGCTACTATGTCTTT
AGGAAAATCTGGAGGAGGAGGATCTGGAGGAGGAGGATCTGGAGGAGGAGGATCTAGAA
ATTTACCTGTAGCTACTCCTGATCCTGGAATGTTCCCTTGTTTACATCATTCTCAAAATTTAT
TAAGAGCTGTATCTAATATGTTACAAAAAGCTAGACAAACTTTAGAATTCTACCCTTGTACTT
CTGAAGAAATTGATCATGAAGATATTACTAAAGATAAAACTTCTACTGTAGAAGCATGTTTAC
CTTTAGAATTAACTAAAAATGAATCTTGTTTAAATTCTAGAGAAACTTCTTTCATTACTAATGG
ATCTTGTTTAGCTTCTAGAAAAACTTCTTTCATGATGGCTTTATGTTTATCTTCTATTTACGAA
GATTTAAAAATGTACCAAGTAGAATTCAAAACTATGAATGCTAAATTATTAATGGATCCTAAA
AGACAAATTTTCTTAGATCAAAATATGTTAGCTGTAATTGATGAATTAATGCAAGCATTAAAT
TTCAATTCTGAAACTGTACCTCAAAAATCTTCTTTAGAAGAACCTGATTTCTACAAAACTAAA
ATTAAATTATGTATTTTATTACATGCTTTCAGAATTAGAGCTGTAACTATTGATAGAGTAATG
TCTTACTTAAATGCTTCT
GOI#15 rhLIF
(SEQ ID NO. 17)
ATGGTATTATCTGGAGCTTTATGTTTCAGAATGAAAGATTCTGCTTTAAAAGTATTATACTTA
CATAATAATCAATTATTAGCTGGAGGATTACATGAAGGAAAAGTAATTAAAGGAGAAGAAAT
TTCTGTAGTACCTAATAGATGGTTAGATGCTTCTTTATCTCCTGTAATTTTAGGAGTACAAG
GAGGATCTCAATGTTTATCTTGTGGAGTAGGACAAGAACCTACTTTAACTTTAGAACCTGTA
AATATTATGGAATTATACTTAGTAGCTAAAGAATCTAAATCTTTCACTTTCTACAGAAGAGAT
ATGGGATTAACTTCTTCTTTCGGATCTGCTGCTTACCCTGGATGGTTCTTATGTACTGTACC
TGAAGCTGATCAACCTGTAAGATTAACTCAATTACCTGAAAATGGAGGATGGAATGCTCCTA
TTACTGATTTCTACTTCCAACAATGTGAT
GOI#16 rhIL-4
(SEQ ID NO. 18)
ATGCATAAATGTGATATTACTTTACAAGAAATTATTAAAACTTTAAATTCTTTAACTGAACAAA
AAACTTTATGTACTGAATTAACTGTAACTGATATTTTCGCTGCTTCTAAAAATACTACTGAAA
AAGAAACTTTCTGTAGAGCTGCTACTGTATTAAGACAATTCTACTCTCATCATGAAAAAGAT
ACTAGATGTTTAGGAGCTACTGCTCAACAATTCCATAGACATAAACAATTAATTAGATTCTTA
AAAAGATTAGATAGAAATTTATGGGGATTAGCTGGATTAAATTCTTGTCCTGTAAAAGAAGC
TAATCAATCTACTTTAGAAAATTTCTTAGAAAGATTAAAAACTATTATGAGAGAAAAATACTC
TAAATGTTCTTCT
GOI#17 rhBMP2
(SEQ ID NO. 19)
ATGCAAGCTAAACATAAACAAAGAAAAAGATTAAAATCTTCTTGTAAAAGACATCCTTTATAC
GTAGATTTCTCTGATGTAGGATGGAATGATTGGATTGTAGCTCCTCCTGGATACCATGCTTT
CTACTGTCATGGAGAATGTCCTTTCCCTTTAGCTGATCATTTAAATTCTACTAATCATGCTAT
TGTACAAACTTTAGTAAATTCTGTAAATTCTAAAATTCCTAAAGCATGTTGTGTACCTACTGA
ATTATCTGCTATTTCTATGTTATACTTAGATGAAAATGAAAAAGTAGTATTAAAAAATTACCAA
GATATGGTAGTAGAAGGATGTGGATGTAGA
GOI#18 rhBMP7
(SEQ ID NO. 20)
ATGTCTACTGGATCTAAACAAAGATCTCAAAATAGATCTAAAACTCCTAAAAATCAAGAgGCT
TTAAGAATGGCTAATGTAGCTGAAAATTCTTCTTCTGATCAAAGACAgGCTTGTAAAAAACAT
GAATTATACGTATCTTTCAGAGATTTAGGATGGCAAGATTGGATTATTGCTCCTGAAGGATA
CGCTGCTTACTACTGTGAAGGAGAATGTGCTTTCCCTTTAAATTCTTACATGAATGCTACTA
ATCATGCTATTGTACAAACTTTAGTACATTTCATTAATCCTGAAACTGTACCTAAACCTTGTT
GTGCTCCTACTCAATTAAATGCTATTTCTGTATTATACTTCGATGATTCTTCTAATGTAATTTT
AAAAAAATACAGAAATATGGTAGTAAGAGCTTGTGGATGTCAT
GOI#19 rhTGFB1
(SEQ ID NO. 21)
ATGGCTTTAGATACTAATTACTGTTTCTCTTCTACTGAAAAAAATTGTTGTGTAAGACAATTA
TACATTGATTTCAGAAAAGATTTAGGATGGAAATGGATTCATGAACCTAAAGGATACCATGC
TAATTTCTGTTTAGGACCTTGTCCTTACATTTGGTCTTTAGATACTCAATACTCTAAAGTATT
AGCTTTATACAATCAACATAATCCTGGAGCTTCTGCTGCTCCTTGTTGTGTACCTCAAGCTT
TAGAACCTTTACCTATTGTATACTACGTAGGAAGAAAACCTAAAGTAGAACAATTATCTAATA
TGATTGTAAGATCTTGTAAATGTTCT
GOI#20 rhFSH
(SEQ ID NO. 22)
ATGAATTCTTGTGAATTAACTAATATTACTATTGCTATTGAAAAAGAAGAATGTAGATTCTGT
ATTTCTATTAATACTACTTGGTGTGCTGGATACTGTTACACTAGAGATTTAGTATACAAAGAT
CCTGCTAGACCTAAAATTCAAAAAACTTGTACTTTCAAAGAATTAGTATACGAAACTGTAAG
AGTACCTGGATGTGCTCATCATGCTGATTCTTTATACACTTACCCTGTAGCTACTCAATGTC
ATTGTGGAAAATGTGATTCTGATTCTACTGATTGTACTGTAAGAGGATTAGGACCTTCTTAC
TGTTCTTTCGGAGAAATGAAAGAAGGAGGAGGATCTGGAGGAGGATCTGGAGGAGGATCT
GGAGGAGGAGCTCCTGATGTACAAGATTGTCCTGAATGTACTTTACAAGAAAATCCTTTCTT
CTCTCAACCTGGAGCTCCTATTTTACAATGTATGGGATGTTGTTTCTCTAGAGCTTACCCTA
CTCCTTTAAGATCTAAAAAAACTATGTTAGTACAAAAAAATGTAACTTCTGAATCTACTTGTT
GTGTAGCTAAATCTTACAATAGAGTAACTGTAATGGGAGGATTCAAAGTAGAAAATCATACT
GCTTGTCATTGTTCTACTTGTTACTACCATAAATCT

### Example 2. Production of Transformation Vectors and Expression Cassettes of the Present Invention

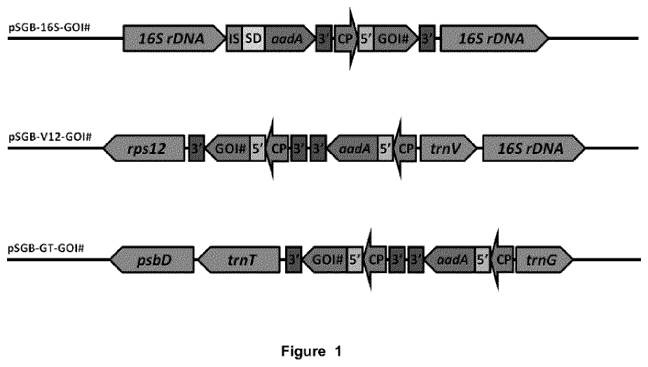

Transformation DNA constructs (vectors) of FIG. 1 were designed to integrate an appropriate expression cassette into the intergenic spacers between i) trnl and trnA genes of the rm16 operon; ii) rps12 and trV genes of the tobacco plastome NC_001879 or tomato plastome NC_007898; and iii) trnT and trnG genes of the Chrysanthemum indicum plastome (NC_020320);
i) DNA sequences between nucleotides 103473-105395 and 105396-106485 of the tobacco plastome (NC_001879) were used as the respective left and right flanking sequences of the expression cassettes in constructs “$16”;
ii) DNA sequences between nucleotides 100162-101709 and 101710-103183 of the tobacco plastome (NC_001879) were used as the respective left and right flanking sequences of the expression cassette in constructs “V12”;
iii) DNA sequences between nucleotides 29266-30774 and 30775-32087 of the Chrysanthemum indicum plastome (NC_020320) were used as the respective left and right flanking sequences of the expression cassette in constructs “GT”;

The following cis-linked genetic elements can be used to comprise plastid expression cassettes of various configurations to engineer and create prolific bioreactor lines:

### Intergenic Spacers (IS):

DNA sequence fragment homologous to nucleotides 13630-13702 (73 base pairs) found between the genes atpH and atpF in the tobacco plastome (NC_001879): (SEQ ID NO. 23)

5′-TTAGCATACTGACTCGCTTTCATCCTTCCCGTTCATAGACCAAGGGA
AACTCTTTTTAGTAAGTGTTAGTGTT-3′;

DNA sequence fragment homologous to nucleotides 77030-77095 (66 base pairs) found between the genes psbN and psbH in the tobacco plastome (NC_001879): (SEQ ID NO. 24)

5′-TCTATGATAGGATCGTTTATTTACAACGGAATGGTATACAAAGTCAA
CAGATCTCAATGAATACAA-3′;

DNA sequence fragment homologous to nucleotides 16043-16115 (73 base pairs) found between the genes rps2 and atpl in the tobacco plastome (NC_001879): (SEQ ID NO. 25)

5′-AGTATTCTAAATCTTAGTTGGTATTCAAAATATCCGATTCAAGTAGA
CAAAGAGATGGTTGAATCAAAAAATT-3′;

DNA sequence fragment homologous to nucleotides 16943-17015 (73 base pairs) found between the genes rpoC2 and rps2 in the tobacco plastome (NC_001879): (SEQ ID NO. 26)

5′-CAGTTCAGGGTTCCTCGTCTCTTTTTTTTTTTTTGAAAAAGAATAA
AAAAAAAAGGGGGGGGTGTGGAGAGAA-3′;

### Untranslated 5′ Sequences (5′UTRs):

Untranslated sequence found on the 5′ end of the plastid psbA gene of the Nicotiana tobacum plastome (NC_001879, complementary nucleotides 1598-1675): (SEQ ID NO. 27)

5′-CTTCCATTTTCTATTTTGATTTGTAGAAAACTAGTGTGCTTGGGAG
TCCCTGATGATTAAATAAACCAAGATTTTACC-3′;

Untranslated sequence found on the 5′ end of the phage 77 G10 gene (NC_001604.1; nucleotides 22882-22966): (SEQ ID NO. 28)

5′-GAAATTAATACGACTCACTATAGGGAGACCACAACGGTTTCCCTCT
AGAAATAATTTTGTTTAACTTTAAGAAGGAGATATACAT-3′;

Untranslated sequence found on the 5′ end of the rbcL gene of the Nicotiana tobacum plastome (NC_001879, nucleotides 57543-57599): (SEQ ID NO. 29)

5′-TCGAGTAGACCTTGTTGTTGTGAGAATTCTTAATTCATGAGTTGTA
GGGAGGGATTT-3′;

### Untranslated 3′ Sequences (3′UTRs):

Untranslated 3′ sequence found on the end of the psbC gene of the Populus alba plastome (NC_008235, nucleotides 34875-to-35052): (SEQ ID NO. 30)

5′-TTGAGAAAAGAGATCCAATAGTTGAAGTAGGAATCCATTTGATTCT
ACTATACATATTGATTGGGTCGATCAGGTCATACTTAGAGAGTCTGCCT
TTTTCCTTTCTTTTCAACTCATTTAGATTTAGATCTAATCGATTTTTTT
ATGGCTCGGCTATCCTACCTAGTCGAGCCATTCA-3′;

Untranslated sequence found on the 3′ end of the rbcL gene of the Populus alba plastome (NC_008235, nucleotides 56790-57022): (SEQ ID NO. 31)

5′-TCCACTAATTAATGTTCATTCTCGTAATTGAATTGCAATTAAATTA
AACTCGGCCCAATCTTTTACTAAAAGGATTGAGCCGAATACAAATAGCC
TATTGTATATATTTGTATATATTTTTGATAGATACATACTTAGCTAAAT
ATACAAGATCTTAAATACAAAATATAAGACTCAAAGAAATCAAACCTTT
CTACTCTTGTCTTAGATCCCTAATTAATCCTAAGGATGTG-3′;

Untranslated sequence found on the 3′ end of the psbA gene of the Medicago truncatula plastome (NC_003119.8, nucleotides 123553-123720): (SEQ ID NO. 32)

5′-GATTTTGGTTTTCAAAAAGGATACGCGTTTTTGAAAATAAAGGGGT
AAAGGAGTAATATCAACATTGTTGATATTACTCCCCCTTTTACTTTTTG
TTAGTAGTCTTTTTCTGTATGCAATACATATACAGAAATTAATCAATTA
TTTATTAAGTTCATTTAGCATTTT-3′;

Untranslated DNA 3′ end sequence fragment (320 base pairs) of the E. coli threonine attenuator characterized as an efficient transcription terminator in spinach chloroplasts (Chen and Orozco, 1988; Gardner, 1982): (SEQ ID NO. 33)

5′-GGATCCTCAACTGTGAGGAGGCTCACGGACGCGAAGAACAGGCACG
CGTACAGGAAACACAGAAAAAAGCCCGCACCTGACAGTGCGGGCTTTTT
TTTTCGACCAAAGGTAACGAGGTAACAACCATGCGAGTGTTGAAGTTCG
GCGGTACATCAGTGGCAAATGCAGAACGTTTTCTGCGTGTTGCCGATAT
TCTGGAAAGCAATGCCAGGCAGGGGCAGGTGGGGGAATTCACTGGCCGT
CGTTTTACAACGTCGTGACTGGGAAAACCCTGGCGTTACCCAACTTAAT
CGCCTTGCAGCACATCCCCCTTTCGCCAG-3′;

### Core Promoter Sequences (CP):

DNA sequence fragment homologous to complimentary nucleotides 1676-1737 (62 base pairs) of the tobacco plastome (NC_001879) containing the core promoter of the plastid psbA gene: (SEQ ID NO. 34)

5′-GATCTACATACACCTTGGTTGACACGAGTATATAAGTCATGTTATA
CTGTTGAATAACAAGC-3′;

DNA sequence fragment homologous to nucleotides 102565-102648 (83 base pairs) of the tobacco plastome (NC_001879) containing the core promoter of the plastid rm operon (Prmn) engineered with two triplet nucleotide “neutral” mutations (“−58-56” ATG to tac; and “−22-20” AGG to tcc; Suzuki et al, 2003): (SEQ ID NO. 35)

5′-GCTCCCCCGCCGTCGTTCAATGAGAtacGATAAGAGGCTCGTGGGA
TTGACGTGAGGGGGCtccGATGGCTATATTTCTGGGA-3′;

DNA sequence fragment homologous to nucleotides 57378-57418 (41 base pairs) of the tobacco plastome (NC_001879) containing the core promoter of the plastid rbcL gene (PrbcL): (SEQ ID NO. 36)

5′-ATTGGGTTGCGCTATATATATGAAAGAGTATACAATAATGA-3′;

Shine-Dalgarno sequence (SD):

5′-GAAGGAG-3′;

Selection Marker Gene (aadA):

Gene aadA open reading frame encoding aminoglycoside acetyltransferase marker for spectinomycin resistance with a stop codon “TAA”: (SEQ ID NO. 37)

5′-ATGGCTCGTGAAGCGGTTATCGCCGAAGTATCAACTCAACTATCA
GAGGTAGTTGGCGTCATCGAGCGCCATCTCGAACCGACGTTGCTGGCC
GTACATTTGTACGGCTCCGCAGTGGATGGCGGCCTGAAGCCACACAGT
GATATTGATTTGCTGGTTACGGTGACCGTAAGGCTTGATGAAACAACG
CGGCGAGCTTTGATCAACGACCTTTTGGAAACTTCGGCTTCCCCTGGA
GAGAGCGAGATTCTCCGCGCTGTAGAAGTCACCATTGTTGTGCACGAC
GACATCATTCCGTGGCGTTATCCAGCTAAGCGCGAACTGCAATTTGGA
GAATGGCAGCGCAATGACATTCTTGCIGGaATCTTCGAGCCAGCCACG
ATCGACATTGATCTGGCTATCTTGCTGACAAAAGCAAGAGAACATAGC
GTTGCCTTGGTAGGTCCAGCGGCGGAGGAACTCTTTGATCCGGTTCCT
GAACAGGATCTATTTGAGGCGCTAAATGAAACCTTAACGCTATGGAAC
TCGCCGCCCGACTGGGCTGGCGATGAGCGAAATGTAGTGCTTACGTTG
TCCCGCATTTGGTACAGCGCAGTAACCGGCAAAATCGCGCCGAAGGAT
GTCGCTGCCGACTGGGCAATGGAGCGCCTGCCGGCCCAGTATCAGCCC
GTCATACTTGAAGCTAGACAGGCTTATCTTGGACAAGAAGAAGATCGC
TTGGCCTCGCGCGCAGATCAGTTGGAAGAATTTGTCCACTACGTGAAA
GGCGAGATCACCAAGGTAGTCGGCAAATAA-3′;

The specific vectors employed in these studies are as follows (vector backbone (pUC57 Amp^{R}):

### Legend:

Grey (GY)-Flanking sequences
Yellow (Y)-Spacer sequences
Green (GN)-Shine-Dalgarno sequence
Dark Blue (DB)-Core promoter sequences
Light Blue (LB)-aadA sequence
Green-Blue (GB)-5′UTR sequences
Brown (BN)-Lumen-targeting sequences
Purple (PU)-GOI sequences
Pink (PK)-3′UTR sequences

### VECTOR PSGB16S-GOI #1 (IL-38)

(-trnl-^{GY}-psbN-psbH-^{Y}-^{GN}-aadA-^{IB}-TpsbC-^{PK}-PpsbA-^{DB}-17G10-^{GB}-IL-38-^{PU}-TrbcL-^{PK}-tmA-^{GY})

### VECTOR PSGB16S-GOI #3 (IL-37B)

(-trnl-^{GY}-psbN-psbH-^{Y}-^{GN}-aadA-^{LB}-TpsbC-^{PK}-PpsbA-^{DB}-psbA-^{GB}-IL-37b-^{PU}-TrbcL-^{PK}-trnA-^{GY})

### VECTOR PSGB16S-GOI #4 (IL-33)

(-trnl-^{GY}-psbN-psbH-^{Y}-^{GN}-aadA-^{LB}-TpsbC-^{PK}-PpsbA-^{DB}-psbA-^{GB}-IL-33-^{PU}-TrbcL-^{PK}-trnA-^{GY})

### VECTOR PSGB16S-GOI #10 (G-CSF)

(-trnl-^{GY}_psbN-psbH-^{Y}-^{GN}-aadA-^{LB}-TpsbC-^{PK}-PpsbA-^{DB}-psbA-^{GB}-G-CSF-^{PU}-TrbcL-^{PK}-trnA-^{GY})

### Example 3. Expression Cassette Functionality Test

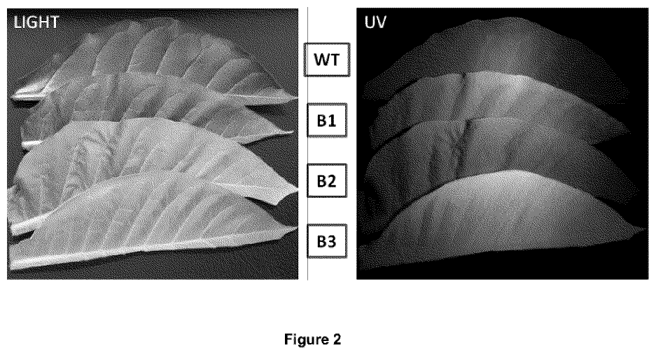

The applicability and functionality of several designed chloroplast expression cassettes were examined by expression of the recombinant Green Fluorescent Protein (GFP, UniProt P42212; Prasher, D. C., Eckenrode, V. K., Ward, W. W., Prendergast, F. G., Cormier, M. J. (1992) Primary structure of the Aequorea victoria green fluorescent protein. Gene 111, 229-233; hereby incorporated by reference) in plastome-engineered tobacco plants (bioreactor lines). Significant yield difference in expression of GFP was confirmed by visual inspection of fluorescence under UV in mature leaves of the plants transformed with different expression cassettes (Table Il; FIG. 2).

TABLE II
Optimization of Different Components of the
Plastid Expression Cassettes Expressing GFP.

| | | | GFP expression |
|---|---|---|---|
| Bioreactor | CodonUsage | 5′ UTR | (% TSP*) |
| B1 | − | − | ~1% |
| B2 | + | − | ~10% |
| B3 | + | + | ~50% |

Optimized rGFP gene with HIS-tag and c-myc tag: (SEQ ID NO. 38)

5′-ATGGCTAGCAGGTCTCATCATCATCATCATCATCATGATAATAAAC
AAGAAAATTTATACTTCCAAGGAAGAAAAGGAGAAGAATTATTCACTGG
AGTAGTACCTATTTTAGTAGAATTAGATGGAGATGTAAATGGACATAAA
TTCTCTGTAAGAGGAGAAGGAGAAGGAGATGCTACTAATGGAAAATTAA
CTTTAAAATTCATTTGTACTACTGGAAAATTACCTGTACCTTGGCCTAC
TTTAGTAACTACTTTAACTTACGGAGTACAATGTTTCGCTAGATACCCT
GATCATATGAAACAACATGATTTCTTCAAATCTGCTATGCCTGAAGGAT
ACGTACAAGAAAGAACTATTTCTTTCAAAGATGATGGAACTTACAAAAC
TAGAGCTGAAGTAAAATTCGAAGGAGATACTTTAGTAAATAGAATTGAA
TTAAAAGGAATTGATTTCAAAGAAGATGGAAATATTTTAGGACATAAAT
TAGAATACAATTTCAATTCTCATAATGTATACATTACTGCTGATAAACA
AAAAAATGGAATTAAAGCTAATTTCAAAATTAGACATAATGTAGAAGAT
GGATCTGTACAATTAGCTGATCATTACCAACAAAATACTCCTATTGGAG
ATGGACCTGTATTATTACCTGATAATCATTACTTATCTACTCAATCTGT
ATTATCTAAAGATCCTAATGAAAAAAGAGATCATATGGTATTATTAGAA
TTCGTAACTGCTGCTGGAATTACTCATGGAATGGATGAATTATACAAAG
AACCTGCTCTTGAACAAAAATTAATTTCTGAAGAAGATTTATAA-3′;

The specific vectors that were employed for these studies are as follows (vector backbone (pUC57 Amp^{R}):

### Legend:

Grey (GY)-Flanking sequences
Yellow (Y)-Spacer sequences
Green (GN)-Shine-Dalgarno sequence
Dark Blue (DB)-Core promoter sequences
Light Blue (LB)-aadA sequence
Green-Blue (GB)-5′UTR sequences
Brown (BN)-Lumen-targeting sequences
Purple (PU)-GOI sequences
Pink (PK)-3′UTR sequences

### VECTOR PSGB16S-GFP

(-tml-^{GY}-psbN-psbH-^{Y}-^{GN}-aadA-^{LB}-TpsbC-^{PK}-PpsbA-^{DB}-T7G10-^{GB}-GFP-^{PU}-TrbCL-^{PK}-trnA-^{GY})

### VECTOR PSGB-V12-GFP

(-rps12-^{GY}-TrbcL-^{PK}-GFP-^{PU}-5′T7G10-^{GB}-Prm-^{DE}-Ectha-TpsbC-^{PK}-aadA-^{LB}-psbA-^{GB}-PpsbA-^{DB}-trV-16S-^{GY})

### VECTOR PSGB-GT-GFP

(-trnG-^{GY}-PpsbA-^{DB}-psbA-^{GB}-aadA-^{LB}-TpsbC-Ectha-^{PK}-Prmn-^{DB}-T7G10-^{GB}-GFP-^{PU}-TrbcL-^{PK}-psbD-trnT-^{GY})

As shown in FIG. 2, different expression and accumulation levels of rGFP was observed in leaves of plastome-engineered tobacco plants. Leaves from the wild-type (WT) tobacco, as well as from three different bioreactor lines (B1, B2, B3) appear healthy green under normal light. Under the UV light, different levels of rGFP fluorescence are visible in B1, B2 and B3 leaves, contrasting the strong maroon chlorophyll autofluorescence of the WT leaf.

Next, the TSP content of crude extracts of the WT leaf was compared with B3-rGFP bioreactor using SDS-PAGE. As shown in FIG. 3, extract equivalents of 1 mg of fresh leaf tissue collected from young (Y), mature (M), mature-old (MO) and senescing(S) leaves were separated by electrophoresis and stained to reveal the profile of TSP content. The major band of about 50 kDa present in samples from both WT and B3 genotypes was the RuBisCO large subunit, which usually constitutes about 40% to about 50% of TSP. The major band of 32 kDa found in the B3 bioreactor line represented the recombinant GFP produced in chloroplasts, clearly more abundant than the RuBisCO in M, MO and S leaves. Thus, these findings revealed that the rGFP was the dominant protein product in the extract, constituting up to 60% of TSP in mature leaves (FIG. 3).

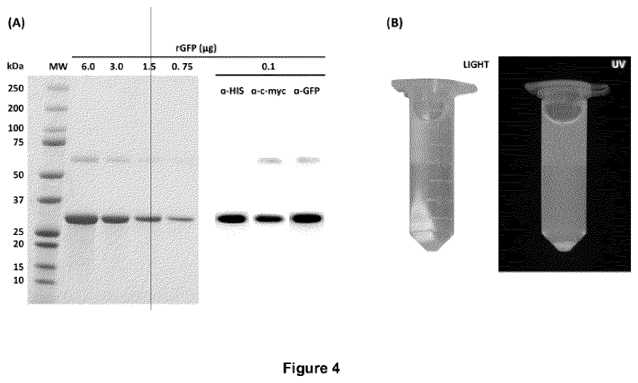

Using the B3 bioreactor line, extraction and manufacturing of up to 4 g of >99% pure recombinant (r) GFP per 1 kg of fresh leaf weight was demonstrated and a strong green florescence was visible under UV light (see FIG. 4). The extraction procedure was based on tissue maceration in ˜3 volumes of extraction buffer, filtration, centrifugation and two rounds of the immobilized metal ion-affinity chromatography (IMAC, FIG. 4). Of note, similar B3-based bioreactor lines were engineered producing 3 additional recombinant fluorescent proteins: Yellow (YFP), Blue (BFP) and Red (RFP), displaying very similar yields (data not shown).

### Example 4. Expression of GOIs in Tobacco Chloroplasts

Twenty genes encoding proteins (DNA sequences shown in Example 1) with increasing complexity of quaternary structure and with a varying number of disulphide bonds were selected and expressed in the form of mature peptides in plastome-engineered tobacco plants' chloroplasts (Table III). Heterodimeric cytokines (IL-35 and FSH) were designed as fusion constructs with a flexible peptide linker (SGGGG) X3 (SEQ ID NO. 39) separating the different domains.

TABLE III
Proteins expressed in tobacco chloroplasts:

| | | | # | | |
|---|---|---|---|---|---|
| Code | | UniProt | Disulphide | Quaternary | |
| # | Protein name | Identifier | bonds** | Structure | Reference |
| 1 | IL-38 | Q8WWZ1 | 0 | Monomer | van de Veerdonk et al., 2012 |
| 2 | IL-11 | P20809 | 0 | Monomer | Harmegnies et al., 2003 |
| 3 | IL-37b | Q9NZH6 | 0 | Monomer & | Kumar et al., 2002 |
| | | | | Homodimer | |
| 4 | IL-33 | O95760 | 0 | Monomer | Hong et al., 2011 |
| 5 | IL-1Ra | P18510 | 1 | Monomer | Latypov et al., 2007 |
| 6 | IL-36Ra | Q9UBH0 | 1 | Monomer | Towne et al., 2011 |
| 7 | IL-2 | P60568 | 1 | Monomer | Stauber et al., 2006 |
| 8 | IL-3 | P08700 | 1 | Monomer | Urdal et al., 1989 |
| 9 | IL-10 | P22301 | 2 | Homodimer | Windsor et al., 1993 |
| 10 | CSF3 | P09919 | 2 | Monomer & | Tamada et al., 2006; |
| | (G-CSF) | | | Trimer | Hill et al., 1993 |
| 11 | IL-13 | P35225 | 2 | Monomer | Lupardus et al., 2010 |
| 12 | FGF19 | O95750 | 2 | Monomer | Harmer et al., 2004 |
| 13 | CSF2 | P04141 | 2 | Monomer | Hansen et al., 2008 |
| | (GM-CSF) | | | | |
| 14* | IL-35 | Q14213 & | 0 & 2 | Heterodimer | Li et al., 2012 |
| | | P29459 | | | |
| 15 | LIF | P15018 | 3 | Monomer | Hinds et al., 1998 |
| 16 | IL-4 | P05112 | 3 | Monomer | Redfield et al., 1994 |
| 17 | BMP2 | P12643 | 4 | Homodimer | Scheufler et al., 1999 |
| | | | (3 + 1) | (Disulphide-linked) | |
| 18 | BMP7 | P18075 | 4 | Homodimer | Swencki-Underwood et al., |
| | | | (3 + 1) | (Disulphide-linked) | 2008 |
| 19 | TGFB1 | P01137 | 5 | Homodimer | Hinck et al., 1996 |
| | | | (4 + 1) | (Disulphide-linked) | |
| 20* | FSH | P01225 & | 6&5 | Heterodimer | Fan & Hendrickson, 2005 |
| | | P01215 | | | |

*Artificial fusion construct;
**Numbers in parenthesis reflect intra- and inter-molecular disulphide bonds, respectively.
Bold indicates proteins approved for clinical use.

### REFERENCES FROM TABLE III

Fan, Q. R. and Hendrickson W. A. (2005) Structure of human follicle-stimulating hormone in complex with its receptor. Nature 433, 269-277;
Hansen, G., Hercus, T. R. Xu, Y., Lopez, A. F., Parker, M. W., and Mckinstry, W. J. (2008) Crystallization and preliminary X-ray diffraction analysis of the ternary human GM-CSF receptor complex. Acta Crystallogr. Sect. F: Struct. Biol. Cryst. Commun. 64, 711-714;
Harmegnies, D., Wang, X. M., Vandenbussche, P., Leon, A., Vusio, P., Grotzinger, J., Jacques, Y., Goormaghtigh, E., Devreese, B. and Content, J. (2003) Characterization of a potent human interleukin-11 agonist. Biochemical Journal 375, 23-32;
Harmer, N. J., Pellegrini, L., Chirgadze, D., Fernandez-Recio, J., Blundell T. L. (2004) The crystal structure of fibroblast growth factor (FGF) 19 reveals novel features of the FGF family and offers a structural basis for its unusual receptor affinity. Biochemistry 43, 629-640;
Hill, C. P. Osslund, T. D, Eisenberg, D. (1993) The structure of granulocyte-colony-stimulating factor and its relationship to other growth factors. Proc. Natl. Acad. Sci. USA, 90, 5167-5171;
Hinck, A. P., Archer, S. J., Qian, S. W., Roberts, A. B., Sporn, M. B., Weatherbee, J. A., Tsang, M. L., Lucas, R., Zhang, B. L., Wenker, J. and Torchia, D. A. (1996) Transforming growth factor beta 1: three-dimensional structure in solution and comparison with the X-ray structure of transforming growth factor beta 2. Biochemistry 35, 8517-8534;
Hinds M G, Maurer T, Zhang J G, et al. (1998) Solution structure of leukemia inhibitory factor. J Biol Chem; 273, 13738-13745;
Hong J, Bae S, Jhun H et al. (2011) Identification of constitutively active IL-33 splice variant. J. Biol. Chem. 286, 20078-20086;
Kumar S, Hanning C R, Brigham-Burke M R, Rieman D J, Lehr R, Khandekar S, Kirkpatrick R B, Scott G F, Lee J C, Lynch F J, Gao W, Gambotto A, Lotze M T (2002) Interleukin-1F7B (IL-1H4/IL-1F7) is processed by caspase-1 and mature IL-1F7B binds to the IL-18 receptor but does not induce IFN-gamma production. Cytokine 18, 61-71;
Latypov, R. F., Harvey, T. S., Liu, D., Bondarenko, P. V., Kohno, T., Fachini, R. A., II, Rosenfeld, R. D., Ketchem, R. R., Brems, D. N., and Raibekas, A. A. (2007) Biophysical characterization of structural properties and folding of interleukin-1 receptor antagonist J. Mol. Biol. 368, 1187-1201;
Li, X., J. Mai, A. Virtue, Y. Yin, R. Gong, X. Sha, S. Gutchigian, A. Frisch, I. Hodge, X. Jiang, et al. (2012) IL-35 is a novel responsive anti-inflammatory cytokine-a new system of categorizing anti-inflammatory cytokines. PLOS ONE 7: e33628;
Lupardus, P. J., Birnbaum, M. E. Garcia, K. C. (2010) Molecular basis for shared cytokine recognition revealed in the structure of an unusually high affinity complex between IL-13 and IL-13R alpha2. Structure 18, 332-342;
Nakamura, M. and Sugiura, M. (2007) Translation efficiencies of synonymous codons are not always correlated with codon usage in tobacco chloroplasts. Plant J. 49, 128-134;
Prasher, D. C., Eckenrode, V. K., Ward, W. W., Prendergast, F. G., Cormier, M. J. (1992) Primary structure of the Aequorea victoria green fluorescent protein. Gene 111, 229-233;
Redfield, C., Smith, L. J., Boyd, J., Lawrence, G. M., Edwards, R. G., Gershater, C. J., Smith, R. A., and Dobson, C. M. (1994) Analysis of the solution structure of human interleukin-4 determined by heteronuclear three-dimensional nuclear magnetic resonance techniques. J. Mol. Biol. 238, 23-41;
Scheufler, C., Sebald, W. and Hulsmeyer, M. (1999) Crystal structure of human bone morphogenetic protein-2 at 2.7 A resolution. J. Mol. Biol. 287, 103-115;
Stauber, D. J. Debler, E. W, Horton, P. A, Smith, K. A, Wilson, I. A. (2006) Crystal structure of the IL-2 signaling complex: paradigm for a heterotrimeric cytokine receptor. Proc. Natl. Acad. Sci. USA, 103, 2788-2793;
Tamada. T., Honjo, E., Maeda, Y., Okamoto, T., Ishibashi, M., Tokunaga, M. & Kuroki, R. (2006) Homodimeric cross-over structure of the human granulocyte colony-stimulating factor (GCSF) receptor signaling complex. Proc. Natl. Acad. Sci. USA, 103, 3135-3140;
Towne, J. E. Renshaw, B. R., Douangpanya, J., Lipsky, B. P., Shen, M., Gabel, C. A., et al. (2011) Interleukin-36 (IL-36) ligands require processing for full agonist (IL-36alpha, IL-36beta, and IL-36gamma) or antagonist (IL-36Ra) activity. J. Biol. Chem. 286, 42594-42602;
Swencki-Underwood, B., et al. (2008) Expression and characterization of a human BMP-7 variant with improved biochemical properties. Protein Expression and Purification 57, 312-319;
Urdal D L, Price V, Sassenfield H M, Cosman D, Gillis S, Park L S (1989) Molecular characterisation of colony stimulating factors and their receptors: Human Interleukin-3. Ann. NY Acad. Sci. 554, 167-176;
van de Veerdonk F. L., Stoeckman A. K., Wu G., Boeckermann A. N., Azam T., Netea M. G., Joosten L. A., van der Meer J. W., Hao R., Kalabokis V., Dinarello C. A. (2012) IL-38 binds to the IL-36 receptor and has biological effects on immune cells similar to IL-36 receptor antagonist. Proc. Nat./Acad. Sci. USA, 109, 3001-3005;
Windsor W. T., Syto R., Tsarbopoulos A., Zhang R., Durkin J., Baldwin S., et al. (1993) Disulfide bond assignments and secondary structure analysis of human and murine interleukin-10. Biochemistry 32, 8807-8815.

### Example 5. Transformation and Regeneration of Transplastomic Clones

Tobacco (low-alkaloid cultivar 81V9) plastome transformation and regeneration of transplastomic clones expressing the GOls was carried out by standard biolistic procedures (Svab, Z. and Maliga, P. (1993) High frequency plastid transformation in tobacco by selection for a chimeric aadA gene. Proc. Natl Acad. Sci. USA, 90, 913-917; Lutz, K. A. and Maliga, P. (2007) Transformation of the plastid genome to study RNA editing. Methods Enzymol. 424, 501-518; Table IV); Two rounds of regeneration (IR, IIR) followed by the rooting of the regenerated clones were performed, all on selective media containing 500 mg/L spectinomycin.

TABLE IV
Results of biolistic transformation* of 81V9 leaves with pSGB-16S-GOI# constructs

| | | | # Green Calli | | Expression | |
|---|---|---|---|---|---|---|
| | | UniProt | Regenerated/ | # PCR - | detected | |
| GOI # | Protein name | Identifier | Checked | positive | (IR Western) | Potted** |
| 1 | IL-38 | Q8WWZ1 | 14/5 | 4 | Y | Y |
| 2*** | IL-11 | P20809 | 21/4 | 2 | N | — |
| 3 | IL-37b | Q9NZH6 | 10/7 | 6 | Y | Y |
| 4 | IL-33 | O95760 | 7/5 | 3 | Y | Y |
| 5*** | IL-1Ra | P18510 | 2/2 | 0 | — | — |
| 6*** | IL-36Ra | Q9UBH0 | 1/1 | 0 | — | — |
| 7 | IL-2 | P60568 | 3/3 | 2 | Y | Y |
| 8 | IL-3 | P08700 | 4/3 | 1 | N | — |
| 9 | IL-10 | P22301 | 8/7 | 4 | N | — |
| 10 | CSF3 (G-CSF) | P09919 | 11/5 | 5 | Y | Y |
| 11 | IL-13 | P35225 | 8/6 | 4 | N | — |
| 12*** | FGF19 | O95750 | 13/10 | 7 | N | — |
| 13 | CSF2 (GM-CSF) | P04141 | 6/6 | 4 | N | — |
| 14 | IL-35 | Q14213 & P29459 | 4/4 | 3 | N | — |
| 15 | LIF | P15018 | 7/5 | 4 | Y | — |
| 16 | IL-4 | P05112 | 4/3 | 3 | Y | — |
| 17 | BMP2 | P12643 | 11/5 | 4 | N | — |
| 18 | BMP7 | P18075 | 9/3 | 2 | N | — |
| 19*** | TGFB1 | P01137 | 4/4 | 3 | N | — |
| 20 | FSH | P01225 & P01215 | 9/4 | 3 | N | — |

*Five leaves were bombarded with each construct;
**Able of autotrophic growth in greenhouse. At least two independent clones per construct were selected for rooting in soil to produce seed.
***Two rounds of bombardment were performed due to lack of transformants after the first round.

### Example 6. GOI Expression in Primary Transformants

As shown in FIG. 5, expression of the GOIs in the plate-harvested, originally-regenerated clones was examined by SDS-PAGE and Western blotting of their extracts of soluble proteins. The originally regenerated clones (Regeneration, IR) were placed in the selective medium after the transformation with pSGB-16S-GOI #constructs. Pairs of extracts for two independent clones (C1, C2) for each GOI genotype were tested and are shown in the Western Blot of FIG. 5. The successfully expressed proteins displayed multimerization patterns even in denaturing conditions of the sample preparation.

### Example 7. Purification with Anti-c-Myc Resin

The expressed recombinant proteins of Example 6, namely, purified samples from clones expressing GOI #1 (rhIL-38), GOI #3 (rhIL-37b), GOI #4 (rhIL-33), GOI #7 (rhIL-2) and GOI #10 (rhG-CSF) (lanes 2-6, respectively) of FIG. 5, were purified using the c-myc-tagged protein mild purification kit ver.2 (MBL International), using about 500 mg leaf tissue harvested from IR clones. As shown in FIG. 6, purified proteins were separated by SDS-PAGE and visualized by staining. This revealed that the previously observed multimers were purified along with the monomeric cytokines.

### Example 8. Profile of Expression in Mature Plants

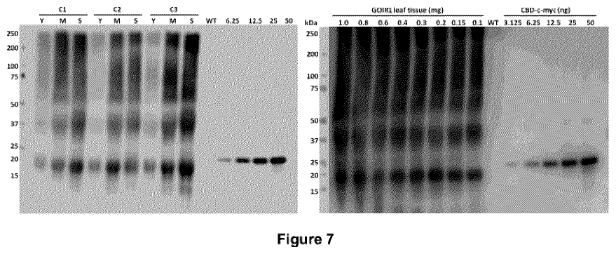

Expression and accumulation levels of the recombinant human cytokines produced in plants (i.e. plantakines) at mature stage was assessed with SDS-PAGE and Western blotting. Extracts of young (Y), mature (M) or senescing(S) leaf tissue from greenhouse-grown IIR clones expressing successful GOIs revealed different spatial accumulation patterns for different recombinant proteins expressed (FIGS. 7-9).

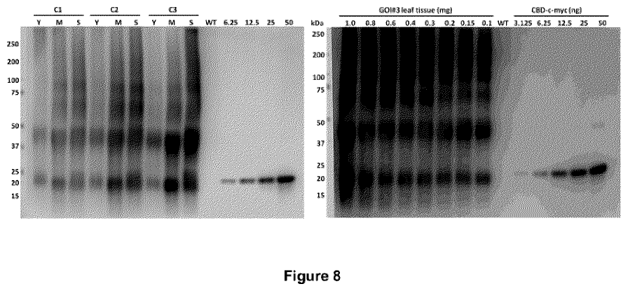

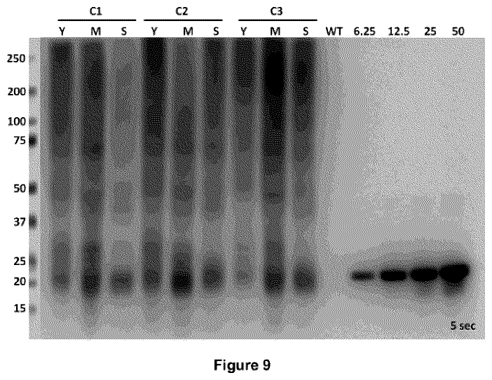

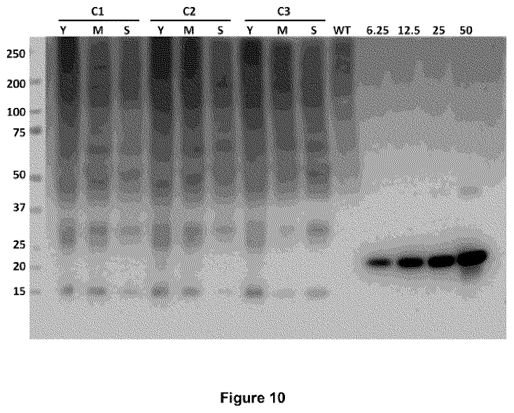

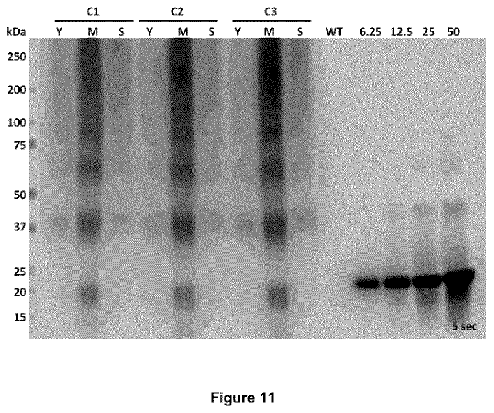

Expression and accumulation patterns for GOI #1 (rIL-38) and GOI #3 (rIL-37b) revealed that the senescing leaves retained the highest yields of those recombinant proteins, accumulating up to 500 ng/mg leaf tissue of GOI #1 and 750 ng/mg leaf tissue of GOI #3 (FIGS. 7 and 8). Expression of GOI #4 (rhIL-33) was detrimentally affected by probable plastid proteolytic activity, as only a minor band of the correct size (27.3 kDa) was detected on the blot among considerable amount of degraded (processed?) protein product (FIG. 9). Minor yields (<5 ng/mg leaf tissue) of the recombinant protein were detected for GOI #7 (rhIL-2)-expressing leaves at all ages tested (FIG. 10). For GOI #10 (G-CSF, FIG. 11) the best yields (>15 ng/mg leaf tissue) were obtained in leaves at mature stage, however, more characterization is needed for these plants as fluctuations in the recombinant protein levels were observed in several extractions, suggesting additional factors affecting expression (probably, diurnal variation, data not shown).

### Example 9. Creating of Bioreactor Lines Expressing His-Tagged Plantakines

Given that the GOls could be successfully expressed as exemplified above, the next step in the investigation was to generate tobacco bioreactor lines expressing and accumulating the identified select cytokines rhIL-38, rhIL-37b and rhG-CSF (i.e. plantakines) tagged with 7×HIS affinity tag for efficient and cost-effective purification. In this experiment, a goal was to produce N-terminal- and C-terminal-tagged versions of the proteins in an effort to improve chances of getting unhindered i) ability to purify those proteins by affinity-chromatography techniques due to possible allosteric effects of the tag placement; and, ii) physiological activity of the purified recombinant cytokines due to the presence of the tag. In addition, this experiment focused on optimizing the purification procedure; characterizing the yields of the purified proteins, production of large amounts of the purified proteins for further characterization of their physiological activity.

Bioreactor lines B2 and B3 bearing genes encoding the selected Plantakines tagged with either N-terminal- and C-terminal 7×HIS tag were created and used to assess expression and accumulation yields (Table V).

TABLE V
Estimated expression and accumulation of rhIL-
38, rhIL-37b and rhG-CSF tagged with 7XHIS affinity
tag using B2 or B3 bioreactor constructs.

| | Estimated |
|---|---|
| Construct-Gene | Expression (g/kg FW) |
| B2-7H-GOI1 | <0.1 |
| B2-7H-GOI3 | <0.1 |
| B2-7H-GOI10 | No expression detected |
| B2-GOI1-7H | No plant regenerated |
| B2-GOI3-7H | >1.0 |
| B2-GOI10-7H | No expression detected |
| B3-7H-GOI1 | <0.1 |
| B3-7H-GOI3 | >1.0 |
| B3-7H-GOI10 | No expression detected |
| B3-GOI1-7H | >1.0 |
| B3-GOI3-7H | <0.1 |
| B3-GOI10-7H | No expression detected |

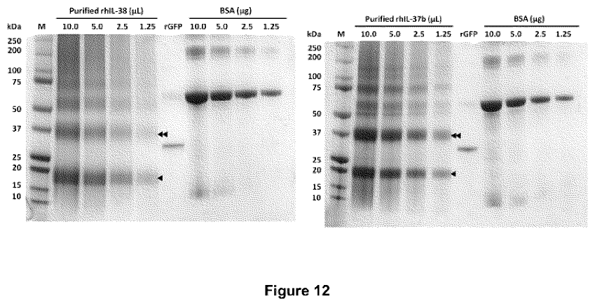

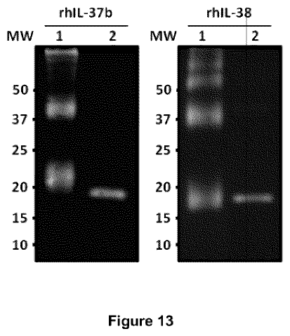

The expressed cytokines (rhIL-38 and rhIL-37) were purified from the leaf tissue extracts of the successful bioreactor lines (B3-GOI1-7H and B2-GOI3-7H) grown in a greenhouse, using an IMAC procedure (FIG. 12). The plant produced IL-38 and IL-37 were compared to recombinant bacterially-produced IL-38 (150 ng, MyBiosource, Cat. #MBS635478: antibody: R&D Biosystems, Cat. #MAB7774) or recombinant bacterially produced IL-37b (150 ng, R&D Biosystems, Cat. #7585-IL-025/CF; antibody: MyBiosource, Cat. #MBS668098), respectively, on the western blot gel (FIG. 13). Based on the results shown therein, the process provided by this Example illustrates a feasibility of affordable production of these potential therapeutic agents in plant bioreactors and subsequent cost-effective IMAC-assisted purification, compared to the c-myc tag-assisted purification procedure demonstrated previously (Example 7). However, a number of contaminating plant proteins were still observed in the purified samples, thus, the purity of the produced recombinant proteins remains to be further improved (FIGS. 12, 13). Assessment of yields of the in planta-produced recombinant human cytokines after one round of IMAC purification from crude extracts estimates production of −0.75 g for rhIL-38 and ˜ 1 g for rhIL-37 per 1 kg of fresh leaf tissue.

### Example 10. Production of Recombinant Protein a

Based on the ability to produce the proteins of interest of the present invention (Example 8-9), further experiments were conducted to develop a tobacco bioreactor line producing Staphylococcus aureus recombinant protein A-a ligand broadly used as the industry standard for purification of antibodies.

Staphylococcus aureus protein A (StpA, UniProt-P02976) is a standard ligand used by the industry for purification of various therapeutic antibodies and contributes significantly to the prohibitive costs of the current manufacturing process. In this example, the expression of an engineered variant of StpA in transplastomic tobacco using both the B2 and B3 bioreactor cassettes was tested. The peptide was engineered to comprise 5 lgG-binding domains E-D-A-B-C of the native protein with a molecular mass of ˜35.4 kDa, containing the C-terminal 7×HIS tag for cost-effective purification, the tag was capped with a cystein residue constituting the C-terminal amino acid of the peptide for efficient and spatially-oriented conjugation to the chromatography resin particles.

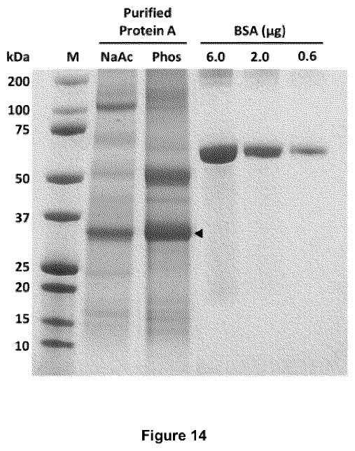

Fresh leaf tissue from B3-StpA transplastomic genotypes, grown in greenhouse, was extracted, purified using an IMAC procedure, and ran on an SDS-PAGE gel. Characterization of the regenerated plants revealed that there was no StpA expression from B2, however, B3-StpA transformants produced a tobacco bioreactor line that accumulated recombinant protein A in chloroplasts at ˜250 mg/kg of fresh leaf biomass. IMAC-assisted purification from crude extracts demonstrated capacity of producing ˜125 mg of the recombinant StpA from 1 kg of extracted fresh leaf tissue. It is noted that although the use of NaAc buffer helped to significantly reduce the amount of RuBisCO (a major contaminating protein of ˜50 kDa, present in the phosphate buffer extract), it also reduced the amount of the purified protein A. Additional contaminating proteins were also detectable in the eluted fractions from both extraction buffers after one round of the IMAC procedure. As shown in FIG. 14, one cycle of IMAC demonstrated insufficient purity levels of the produced recombinant protein A (FIG. 14). Further work is being directed to improving the extraction/purification procedure for the plant-produced StpA protein and manufacturing of StpA-conjugated resin for efficient and cost-effective purification of antibodies.

The specific sequence of the vector used in this study is as follows (vector backbone (pUC57 Amp^{R}): Legend:
Grey (GY)-Flanking sequences
Yellow (Y)-Spacer sequences
Green (GN)-Shine-Dalgarno sequence
Dark Blue (DB)-Core promoter sequences
Light Blue (LB)-aadA sequence
Green-Blue (GB)-5′UTR sequences
Brown (BN)-Lumen-targeting sequences
Purple (PU)-GOI sequences
Pink (PK)-3′UTR sequences

### VECTOR PSGB16S-STPA (PROTEIN A)

(-trnl-^{GY}-psbN-psbH-^{Y}-^{GN}-aadA-^{LB}-TpsbC-^{PK}-PpsbA-^{DB}-T7G10-^{GB}-stpA-^{PU}-TrbcL-^{PK}-trnA-^{GY})

### Example 11. Modulation of Inflammatory Agent-Induced Human PBMCs Responses by Recombinant Human Cytokines IL-37b and IL-38 Produced in Plants Abstract

Affordable therapeutics are vitally needed for humans worldwide. Plant-based production of recombinant proteins can potentially enhance, back-up, or even substitute for the manufacturing capacity of the conventional, fermenter-based technologies. We plastome-engineered a tobacco cultivar to express high levels of two “plantakines”-recombinant human cytokines, interleukins IL-37b and IL-38, and confirmed their native conformation and folding. Assessment of their biological functionality was performed ex vivo by analyzing the effects exerted by the plantakines on levels of 11 cytokines secreted from human Peripheral Blood Mononuclear Cells (PBMCs) challenged with an inflammatory agent. Application of the plant-produced IL-37b and IL-38 in PBMCs stimulated with Lipopolysaccharide or Phytohaemagglutinin resulted in significant, dose-dependent modulation of pro-inflammatory cytokines secretion and attenuation of levels of several cytokines involved in inflammatory response. Our results demonstrate feasibility of manufacturing functional recombinant human proteins using scalable, cost-effective and eco-friendly plant-based bioreactors.

### INTRODUCTION

Plants make a lot of sense as production platforms for all kinds of biologics. Photosynthetic capacity allowing autotrophic growth renders plants the most energy-efficient and cost-effective platform for manufacturing of various recombinant proteins, secondary metabolites and other assorted small molecules, as plants require only three abundantly available raw input ingredients for biosynthesis-carbon dioxide, water and sunlight. Hence, the initial part of the manufacturing process, the “upstream production” that generates the biomass accumulating the desired product ensues significant costs savings, eliminating the need for construction, maintenance and operation of fermenter facilities (Xu et al., 2017) (Buyel et al., 2017) (Huebbers and Buyel, 2021). Benefits to “downstream production” steps of the process, where the desired product is extracted and purified are also recognized for plant-based systems, with some of the bottlenecks being addressed in recent studies (Alam et al., 2018) (Gengenbach et al., 2019) (Schillberg and Finnern, 2021a). Additional advantages of exploiting plants as single-use, clean and biodegradable bioreactors for production of recombinant proteins include inherent safety due to inability of mammalian pathogens to propagate in plant tissue and virtually unlimited scalability of plant-based production (McNulty et al., 2020) (Shanmugaraj et al., 2020).

Since the emergence of the first reports of successful genetic transformation of plants and the expression of recombinant heterologous proteins of human origin in transgenic plants, tremendous technological advances were achieved in the “molecular pharming” field, with the first FDA-approved pharmaceutical for human use in 2012, taliglucerase alfa, produced in carrot cells (Fox, 2012). Today several biopharmaceuticals on the market are sourced from plants and a few biotechnology companies around the world use plant-based production platforms in their manufacturing processes (Ward et al., 2020) (Huebbers and Buyel, 2021) (Schillberg and Finnern, 2021b). Plant-based bioreactors could facilitate making more affordable many biologic drugs in use today and provide a source of therapeutics supplied locally, which can be very beneficial in the context of developing nations, or when global supply chains are disrupted (Schillberg and Finnern, 2021b) (Tsekoa et al., 2020).

Among the methodologies used for plant-based recombinant protein manufacturing, plastome-engineered plants possess several advantageous features as a platform, simply generating extraction-ready biomass from seed. Plastome-engineered plants can express and accumulate very high yields of the desirable product and, thus, can represent the most cost-effective production route (Ahmad et al., 2016) (Adem et al., 2017) (Maliga and Bock, 2011) (Daniell et al., 2021). The goal of the experiments was to demonstrate the feasibility of plastome-engineered plant bioreactor platform for production of biologically active recombinant human cytokines. Based on preliminary screens searching for valuable proteins with a potential for prolific expression in plastids, the plastome of a low-alkaloid tobacco cultivar was designed to produce “bioreactor lines” expressing mature forms of two “plantakines”-human interleukins IL-37 (isoform b, IL-37b) and IL-38, both characterized as anti-inflammatory cytokines (Cavalli and Dinarello, 2018) (Han et al., 2020). IL-37b and IL-38 belong to the IL-1 family of 11 interleukins, 7 of which are pro-inflammatory (Palomo et al., 2015). Both IL-37b and IL-38 function in regulation/mitigation of human inflammatory responses; a plethora of studies demonstrated central involvement for IL-37b and IL-38 in immunity and disease and, therefore, as potential candidates for development as therapeutic agents (Dinarello et al., 2016) (Xu and Huang, 2018). The created plastome-engineered bioreactor lines produced up to ˜1 gram of the recombinant protein per 1 kg of fresh leaf biomass. After confirmation of their correct folding, the biological activity of the plant-produced IL-37b and IL-38 was assessed in ex vivo experiments by monitoring the response to inflammatory agents (IAs) in freshly isolated cultured human Peripheral Blood Mononuclear Cells (PBMCs), manifested in the levels of secreted inflammatory cytokines.

PBMCs are the central and crucial components of the immune system that brings forth a response to intruder pathogens, as well as identifies and fights own body cells that have undergone malignant transformation (cancer). PBMCs are an assorted mixture of highly specialized immune cells, PBMCs population is comprised of a multitude of immune cell types including lymphocytes (˜85%), monocytes (˜15%) and dendritic cells (<1%) (Kleiveland and Kleiveland, 2015). In vitro and ex vivo human PBMCs studies are ubiquitous in cell biology and immunology research and an important biotechnological tool in developing new therapeutics and diagnostics (Wettstein et al., 2019) (Ferreira De Mello et al., 2012) (Martínez-Rodríguez et al . . . 2019) (Hartmann et al.) (Oda et al., 2021). It was hypothesized that by monitoring the inflammatory response in IA-stimulated PBMCs that the effects of the plantakines (and their active concentrations) exerted on the levels of specific inflammatory markers could be studied.

Modulation of inflammation responses from PBMCs stimulated with different IAs as a result of treatments with the plant-produced IL-37b and IL-38 is reported. Attenuation of levels of several secreted inflammatory cytokines, generally consistent with the previous reports characterizing the biological activity of IL-37b and IL-38 as anti-inflammatory has been observed. Both plantakines exerted dose-dependent modulations of PBMCs responses, leading at different concentrations to either inhibition or enhancement of secretion of some of the inflammatory markers monitored. In addition, different IAs brought about different magnitude of inflammatory responses reflected in levels of cytokines secreted from the stimulated PBMCs, confirming a similar experimental outcome reported recently. Thus, this study validates applicability of the plant-based production platform for cost-efficient and eco-friendly manufacturing of functional recombinant human cytokines in large quantities.

### Results

### Monomers, Dimers and Multimers of IL-37b and IL-38 Accumulate in Engineered Chloroplasts

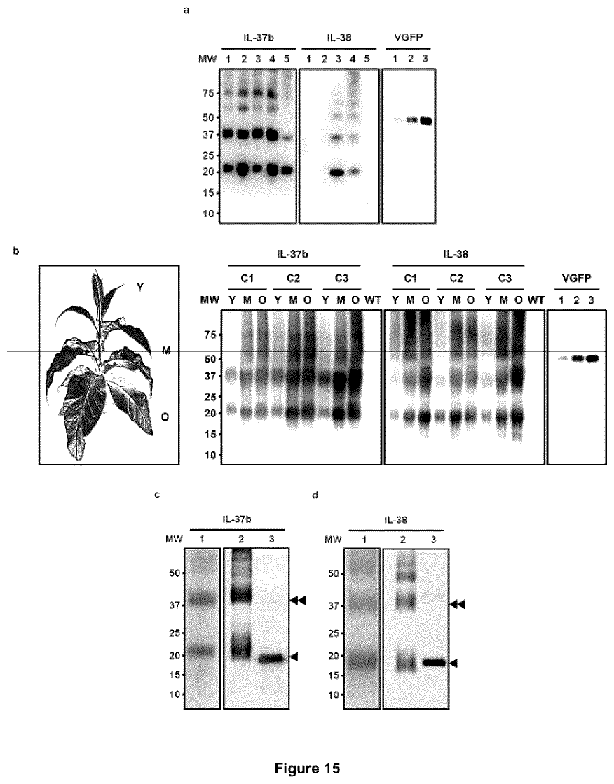

Plastome transformation constructs to produce IL-37b and IL-38 as mature peptides (V46-D218 for IL-37b, C2-W152 for IL-38) were engineered, optimizing the expression by selecting suitable cis-acting regulatory genetic elements and using plastid-preferable codons (data not shown). Screening for prolific producer lines of IL-37b and IL-38 identified the best configurations of plastid expression cassettes by examining their crude leaf tissue extracts with Western blots (FIG. 15a). Two bioreactor lines were selected and grown in greenhouse to maturity, expressing the recombinant human IL-37b and IL-38 at ˜1 g and 0.75 g, respectively, per 1 kg of fresh leaf tissue. Interestingly, prevalent amounts of both plantakines were found to accumulate in older leaves, demonstrating significant stability of these recombinant proteins in the chloroplasts (FIG. 15b). We observed large amounts of the monomeric forms, as well as the dimerized and multimerized forms of the cytokines IL-37b and IL-38 in the crude leaf extracts and in samples after purification; the dimers (and higher molecular weight multimers) were very stable and detectable in SDS-PAGE analyses gels even after harsh denaturing conditions of the sample preparation. That observation was in stark contrast to the bacteria-produced recombinant IL-37b and IL-38 counterparts available commercially, that predominantly presented the monomeric forms of the cytokines when used as controls in Western blot experiments using specific antibodies (FIG. 15c, d). Placement of the HIS-tag at the N-terminal had no effect on the formation of dimers/multimers for both expressed cytokines (data not shown).

### Plantakines Bioactivity Assessment-Experimental Design

The bioactivity of our plantakines IL-37b and IL-38 was assessed by monitoring the secretion of 11 cytokines, generally regarded as inflammatory markers, from PBMCs stimulated with an IA. Freshly isolated human PBMCs were subjected to various treatments-combinations of the IAs with the plantakines at different concentrations. Two different model IAs were used, either the bacterial lipopolysaccharides (LPS) or a lectin from Phaseolus vulgaris (phytohaemagglutinin, PHA); each of the IAs was applied onto cells separately, each IA was applied at two concentrations: LPS at 150 and 300 μg/mL; PHA at 5 and 10 μg/mL. Each IA at each concentration was applied in combination with one of the two plantakines, each of them at three different concentrations: 1, 10 and 100 ng/ml of the monomeric forms present in the purified extracts. Also included were treatments comprised of either IA at their lower concentrations, in combination with both plantakines at 10 ng/ml concentration in order to assess possible synergistic effects. Cells with only IAs applied represented the reference (positive controls for each concentration), cells without any treatment represented the basal level (negative control). The levels of eleven different pro-inflammatory cytokines secreted into the medium from the PBMCs-GM-SCF, IFNγ. TNFα, IL-1α, IL-1β, IL-6, IL-8, IL-22, IL12, IL-17 and IL-10 were compared between the treatments and the controls. We applied Generalized Estimating Equation (GEE) model for the statistical data analysis. This statistical approach allows for nested observations and was used to test the effects of the plantakines IL-37b and IL-38 and their dosage in the context of IA-stimulated PBMCs inflammatory responses.

Different IAs Bring about Different Magnitude of Inflammatory Responses from PBMCs

In order to validate the obtained data, as well as to gain insights into the quantitative and qualitative differences in the PBMCs' inflammatory responses between the two IAs tested, the mean values for each monitored secreted cytokine elicited by either LPS or PHA were first compared. The magnitude of the general responses from the stimulated PBMCs, manifested in levels of the secreted pro-inflammatory cytokines was found significantly different between the two IAs. PHA elicited stronger response in 7 out of 11 pro-inflammatory cytokines monitored; namely, IL-17, IFNγ, TNFα, IL-12, IL-22, IL-10 and IL-6 displayed, respectively, 2930%, 1240%, 55.7%, 118%, 21.5%, 86.4% and 10.1% higher levels, compared with the LPS-elicited levels (p <0.001-p<0.05, FIG. 16). The levels of GM-CSF, IL-8, IL-1a and IL-1B showed no statistically significant difference between the IAs in our experiments. Further dissection of the data focusing on the differences in levels of the inflammatory markers between the lower and higher doses of each IA revealed that the higher dose of PHA elicited higher levels of IFNγ (+249%), IL-1α (+30%), IL-1β (+35%), IL-12 (+65%), IL-17 (+54%), TNFα (+49%) and IL-10 (+51%), compared with the lower dose. Higher dose of LPS brought about higher secreted levels of GM-CSF (+5%), IL-1α (+17%), IL-1β (+20%) and IL-10 (+26%), while secretion of IL-17 decreased (−12%).

Plant-Produced IL-37b and IL-38 Modulate Inflammatory Responses from IA-Stimulated PBMCs

To gain insight into the bioactivity of the plantakines IL-37b and IL-38 exerted on IA-stimulated PBMCs, data generated from treatments with observed modulatory effects on secreted inflammatory markers was compiled. GEE analysis was performed four times, for each combination of the IA and its concentration, for each secreted cytokine monitored, generating statistically significant (p<0.05) level modulations displayed in 118 treatments out of the total 286 treatment combinations assessed. Plantakines exerted statistically significant modulatory effects on the levels of secreted inflammatory cytokines in 67 and 51 treatments that occurred in LPS- and PHA-stimulated PBMCs, respectively (Table VI). Collectively, treatments with plantakines IL-37b and IL-38 resulted in more profound anti-inflammatory activity in LPS-stimulated PBMCs rather than PBMCs stimulated with PHA, as only 10 treatments resulted in increased secretion of inflammatory cytokines in LPS-stimulated PBMCs, while decreased secretion was observed in 57 treatments. In contrast, secretion of inflammatory markers in PHA-stimulated PBMCs was suppressed in 17 treatments with plantakines and increased in 34. Notably, all the treatments with the simultaneous application of both IL-37b and IL-38 brought about increases in secretion of inflammatory cytokines under stimulations with either IA, while separate applications of the plant-produced IL-37b or IL-38 suppressed secretion of inflammatory cytokines in 44 and 30 treatments, and increased it in 12 and 22, respectively. Fewer treatments with plantakines caused suppression of inflammatory cytokines secretion association with a higher concentration of either IA used to stimulate the PBMCs (Table VI).

TABLE VI
Modulation in levels of inflammatory cytokines secreted from IA-stimulated
PBMCs with applied IL-37b and IL-38 plantakines treatments.

| PBMCs stimulation IA | | | | | | | | |
|---|---|---|---|---|---|---|---|---|
| LPS | | | | PHA | | | | |
| 150 pg/mL | | 300 pg/mL | | 5 μg/mL | | 10 μg/mL | | [symbol] A concentration |
| IL-37b | IL-38 | IL-37b | IL-38 | IL-37b | IL-38 | IL-37b | IL-38 | Plantakine treatment |
| 286 | | | | | | | | All treatments combinations |
| 118 | | | | | | | | Treatments with statistically |
| [symbol] 7 | | | | [symbol] | | | | significant modulation (p [symbol] 0.05) |
| 39 | | 28 | | 27 | | 24 | | |
| 10 | | | | 34 | | | | Increased secretion |
| [symbol] * | | [symbol] | | 17** | | 1 [symbol] | | |
| 1 | 1 | 3 | 2 | 3 | 7 | [symbol] | 12 | |
| 57 | | | | 17 | | | | Decreased secretion |
| 34 | | 2 [symbol] | | 1 [symbol] | | 7 | | |
| 18 | 16 | 11 | 12 | 9 | 1 | 6 | 1 | |

*includes 3 treatments combinations with simultaneous application of both plantakines.
**includes 7 treatments combinations with simultaneous application of both plantakines. [symbol]
indicates data missing or illegible when filed

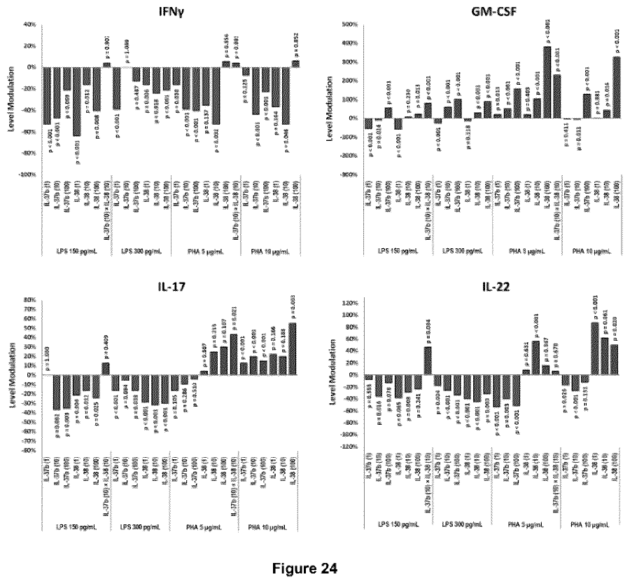

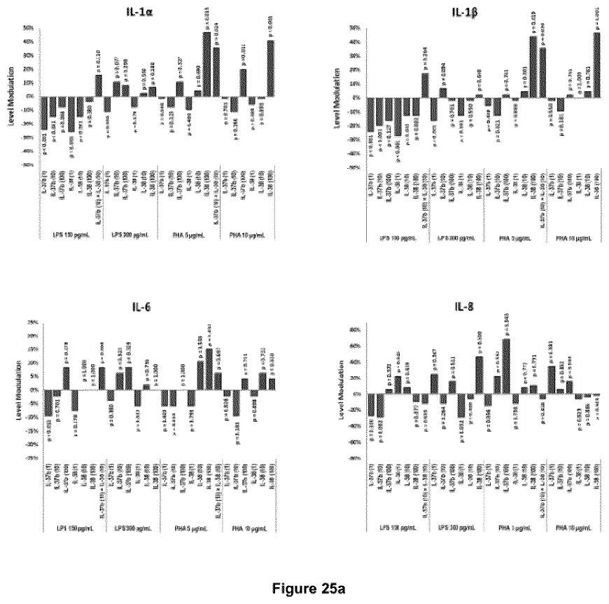

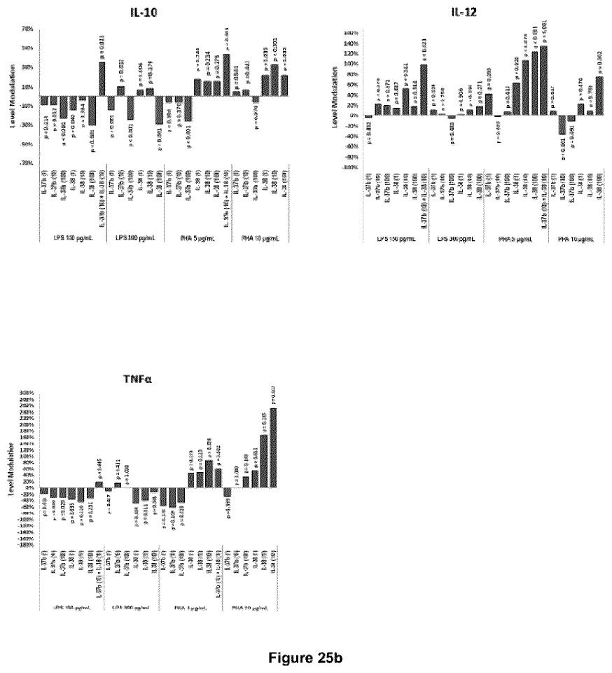

The changes in levels of the secreted inflammatory cytokines from the stimulated PBMCs resulting from treatments with different doses of the plantakines IL-37b and IL-38 were further analyzed. For each inflammatory marker monitored, the outcomes of the treatments were calculated as percentages of secretion modulation with its probability value in comparison with the positive controls at the corresponding IAs concentrations (Table VII; FIG. 24, and FIGS. 25a and 25b). The modulatory effects of the plantakines IL-37b and IL-38 could be observed on the levels of most of the monitored secreted inflammatory cytokines elicited with either LPS or PHA, showing a general tendency of attenuation. IL-37b attenuated levels of IFNγ, IL-1α, IL-1β, IL-22, IL-17 and TNFα in LPS-stimulated PBMCs, the effect could be seen at all the concentrations examined, levels of IFNγ and IL-22 were also reduced by IL-37b in PHA-stimulated PBMCs (Table VII; FIG. 24). Increases in secretion of the inflammatory markers were mostly observed after treatments with higher concentrations of plantakines, whereas 66% of the treatments where secretion increases occurred were associated with the highest dose of plantakines, followed by 28% associated with the intermediate dose and 6% linked to the lowest dose. Unexpectedly, IL-37b at all 3 concentrations brought about an increase in IL-17 secreted from PBMCs stimulated with PHA at 10 μg/mL, similar increases were observed for IL-1α and GM-CSF levels with IL-37b at 100 ng/ml. Modulation of GM-CSF levels by both IL-37b and IL-38 displayed dose-dependent character: at low concentrations (1 ng/mL) both plantakines attenuated GM-CSF levels by more than 50% in PBMCs stimulated with 150 μg/mL LPS, while 100 ng/ml plantakines concentration increased the levels of GM-CSF, those increases observed more profoundly at LPS 300 μg/mL concentration (Table VII). Both plantakines boosted GM-CSF in PHA-stimulated PBMCs: at higher concentrations (100 ng/mL) IL-37b brought about 155.9% and 127.8% increases in GM-CSF levels at 5 μg/mL and 10 μg/mL PHA stimulation, respectively, and IL-38 showed 380.5% and 326.6%, p<0.001. Interesting, a combination of both plantakines, each at concentration 10 ng/ml exerted a 228.6% (p<0.001) increase in secreted GM-CSF levels, pointing out a possible synergistic effect from the simultaneous application, since when applied separately on PBMCs with the same 5 g/mL PHA stimulation, plantakines IL-37b and IL-38 modulated GM-CSF levels to increase 50.5% and 103.3%, respectively (p<0.001). Simultaneous applications of both plantakines resulted in increased secretion of several pro-inflammatory cytokines from PHA-stimulated PBMCs, IL-1α, IL-1β, IL-12, IL-17, TNFα and IL-10 displayed, respectively, 35.0%, 35.0%, 134.5%, 43.3%, 61.6%, and 43.3% increased levels (p<0.001-p<0.05). Notably, only insignificant modulation of IL-6 and IL-8 levels was observed, yet, when plantakines IL-37b and IL-38 were applied at the lowest concentration (1 ng/ml), statistically significant attenuation (−9.5% for IL-6 elicited at 150 μg/mL LPS, p=0.012, and −28.5% for IL-8 elicited at 300 μg/mL LPS, p=0.032) was detected, aligned with the anti-inflammatory functions expected from IL-37b and IL-38 (Table VII).

TABLE VII
Results of GEE model analyses of modulation of inflammatory cytokines levels secreted
from IA-stimulated PBMCs with applied treatments of plantakines IL-37b and IL-38.

| | Plantakines | Inflammatory Agent (IA) | | | |
|---|---|---|---|---|---|
| | Treatments | LPS, pg/mL | | PHA, μg/mL | |
| IM | (ng/mL) | 150 | 300 | 5 | 10 |
| GM-CSF | IL-37b (1) | −54.2%, p < 0.001 | −25.3%, p < 0.001 | 19.6%, p = 0.013 | −5.0%, p = 0.411 |
| | IL-37b (10) | −11.3%, p = 0.024 | 59.2%, p < 0.001 | 50.5%, p < 0.001 | −5.8%, p = 0.011 |
| | IL-37b (100) | 53.9%, p < 0.001 | 100.5%, p < 0.001 | 155.9%, p < 0.001 | 127.8%, p < 0.001 |
| | IL-38 (1) | −59.4%, p < 0.001 | −14.1%, p = 0.118 | 18.0%, p = 0.403 | 1.5%, p = 0.881 |
| | IL-38 (10) | 7.3%, p = 0.230 | 29.1%, p < 0.001 | 103.3%, p < 0.001 | 42.6%, p = 0.016 |
| | IL-38 (100) | 21.2%, p = 0.013 | 88.8%, p < 0.001 | 380.5%, p < 0.001 | 326.6%, p < 0.001 |
| | IL-37b (10) × | 78.7%, p < 0.001 | | 228.6%, p < 0.001 | |
| | IL-38 (10) | | | | |
| IFNγ | IL-37b (1) | −53.2%, p < 0.001 | −39.3%, p < 0.001 | −16.5%, p = 0.038 | −7.7%, p = 0.125 |
| | IL-37b (10) | −47.3%, p < 0.001 | 0.0%, p = 1.000 | −39.3%, p < 0.001 | −44.0%, p < 0.001 |
| | IL-37b (100) | −21.3%, p = 0.009 | −13.1%, p = 0.487 | −40.5%, p < 0.001 | −22.9%, p < 0.001 |
| | IL-38 (1) | −63.9%, p < 0.001 | −16.5%, p = 0.006 | −35.6%, p = 0.137 | −36.9%, p = 0.164 |
| | IL-38 (10) | −16.5%, p = 0.012 | −24.4%, p = 0.018 | −53.2%, p = 0.032 | −53.2%, p = 0.046 |
| | IL-38 (100) | −40.5%, p = 0.008 | −21.3%, p < 0.001 | 6.2%, p = 0.856 | 6.2%, p = 0.852 |
| | IL-37b (10) × | 4.1%, p = 0.907 | | 4.1%, p = 0.883 | |
| | IL-8 (10) | | | | |
| IL-1α | IL-37b (1) | −24.4%, p < 0.001 | −11.3%, p = 0.006 | −2.0%, p = 0.648 | −2.0%, p = 0.701 |
| | IL-37b (10) | −14.8%, p < 0.001 | 10.5%, p = 0.077 | −7.7%, p = 0.125 | −11.3%, p = 0.246 |
| | IL-37b (100) | −7.7%, p = 0.068 | 8.3%, p = 0.298 | 10.5%, p = 0.327 | 19.7%, p = 0.012 |
| | IL-38 (1) | −25.9%, p < 0.001 | −7.7%, p = 0.178 | −9.5%, p = 0.480 | −5.8%, p = 0.469 |
| | IL-38 (10) | −14.8%, p < 0.001 | 2.0%, p = 0.550 | 4.1%, p = 0.690 | −2.0%, p = 0.893 |
| | IL-38 (100) | −3.9%, p = 0.380 | 6.2%, p = 0.188 | 46.2%, p = 0.015 | 40.5%, p < 0.001 |
| | IL-37b (10) × | 15.0%, p = 0.110 | | 35.0%, p = 0.024 | |
| | IL-38 (10) | | | | |
| IL-1β | IL-37b (1) | −24.4%, p < 0.001 | −16.5%, p < 0.001 | −5.8%, p = 0.409 | −2.0%, p = 0.550 |
| | IL-37b (10) | −19.7%, p < 0.001 | 6.2%, p = 0.094 | −13.1%, p = 0.021 | −9.5%, p = 0.181 |
| | IL-37b (100) | −16.5%, p = 0.127 | −2.0%, p = 0.701 | 2.0%, p = 0.761 | 2.0%, p = 0.701 |
| | IL-38 (1) | −27.4%, p < 0.001 | −13.1%, p < 0.001 | −2.0%, p = 0.888 | 0.0%, p = 1.000 |
| | IL-38 (10) | −13.1%, p = 0.002 | −2.0%, p = 0,550 | 4.1%, p = 0.801 | 4.1%, p = 0.761 |
| | IL-38 (100) | −13.1%, p = 0.002 | 2.0%, p = 0.648 | 43.3%, p = 0.019 | 46.2%, p = 0.001 |
| | IL-37b (10) × | 17.4%, p = 0.264 | | 35.0%, p = 0.030 | |
| | IL-38 (10) | | | | |
| IL-6 | IL-37b (1) | −9.5%, p = 0.012 | −3.9%, p = 0.380 | −5.8%, p = 0.409 | −2.0%, p = 0.826 |
| | IL-37b (10) | −2.0%, p = 0.701 | 6.2%, p = 0.323 | −5.8%, p = 0.515 | −9.5%, p = 0.181 |
| | IL-37b (100) | 8.3%, p = 0.178 | 8.3%, p = 0.329 | 0.0%, p = 1.000 | 4.1%, p = 0.711 |
| | IL-38 (1) | −7.7%, p = 0.178 | −5.8%, p = 0.323 | −5.8%, p = 0.798 | −2.0%, p = 0.898 |
| | IL-38 (10) | 0.0%, p = 1.000 | 2.0%, p = 0.736 | 10.5%, p = 0.538 | 6.2%, p = 0.722 |
| | IL-38 (100) | 0.0%, p = 1.000 | 0.0%, p = 1.000 | 15.0%, p = 0.432 | 4.1%, p = 0.810 |
| | IL-37b (10) × | 8.3%, p = 0.608 | | 6.2%, p = 0.697 | |
| | IL-38 (10) | | | | |
| IL-8 | IL-37b (1) | −26.7%, p = 0.100 | 24.6%, p = 0.367 | −14.8%, p = 0.356 | 35.0%, p = 0.391 |
| | IL-37b (10) | −28.5%, p = 0.082 | −11.3%, p = 0.264 | 22.1%, p = 0.552 | 6.2%, p = 0.832 |
| | IL-37b (100) | 6.2%, p = 0.370 | 15.0%, p = 0.511 | 68.2%, p = 0.343 | 15.0%, p = 0.544 |
| | IL-38 (1) | 22.1%, p = 0.625 | −28.5%, p = 0.032 | −11.3%, p = 0.736 | −5.8%, p = 0.823 |
| | IL-38 (10) | 8.3%, p = 0.639 | −5.8%, p = 0.469 | 8.3%, p = 0.772 | −3.9%, p = 0.886 |
| | IL-38 (100) | −9.5%, p = 0.077 | 46.2%, p = 0.500 | 10.5%, p = 0.731 | −2.0%, p = 0.943 |
| | IL-37b (10) × | −11.3%, p = 0.655 | | −5.8%, p = 0.818 | |
| | IL-38 (10) | | | | |
| IL-22 | IL-37b (1) | −7.9%, p = 0.555 | −18.2%, p = 0.004 | −54.0%, p < 0.001 | −17.3%, p = 0.026 |
| | IL-37b (10) | −36.0%, p = 0.016 | −26.1%, p < 0.001 | −40.3%, p = 0.003 | −26.4%, p < 0.001 |
| | IL-37b (100) | −13.4%, p = 0.078 | −33.5%, p < 0.001 | −51.2%, p < 0.001 | −12.4%, p = 0.131 |
| | IL-38 (1) | −38.1%, p = 0.065 | −40.0%, p < 0.001 | 8.1%, p = 0.631 | 86.8%, p < 0.001 |
| | IL-38 (10) | −29.4%, p = 0.008 | −45.0%, p < 0.001 | 55.8%, p < 0.001 | 61.7%, p = 0.061 |
| | IL-38 (100) | −23.7%, p = 0.241 | −31.8%, p = 0.003 | 15.3%, p = 0.347 | 50.0%, p = 0.020 |
| | IL-37b (10) × | 46.0%, p = 0.084 | | 5.9%, p = 0.670 | |
| | IL-38 (10) | | | | |
| IL-12 | IL-37b (1) | −4.0%, p = 0.832 | 11.0%, p = 0.534 | 42.1%, p = 0.055 | 9.1%, p = 0.617 |
| | IL-37b (10) | 21.8%, p = 0.574 | 2.8%, p = 0.759 | −2.6%, p = 0.825 | −36.6%, p < 0.001 |
| | IL-37b (100) | 19.7%, p = 0.372 | −4.8%, p = 0.483 | 7.5%, p = 0.613 | −11.0%, p = 0.091 |
| | IL-38 (1) | 14.9%, p = 0.607 | 1.9%, p = 0.906 | 63.3%, p = 0.020 | 22.2%, p = 0.476 |
| | IL-38 (10) | 51.1%, p = 0.522 | 10.6%, p = 0.586 | 107.0%, p = 0.079 | 8.8%, p = 0.750 |
| | IL-38 (100) | 17.2%, p = 0.544 | 17.4%, p = 0.271 | 123.0%, p < 0.001 | 74.3%, p = 0.002 |
| | IL-37b (10) × | 98.5%, p = 0.023 | | 134.5%, p < 0.001 | |
| | IL-38 (10) | | | | |
| IL-17 | IL-37b (1) | 0.0%, p = 1.000 | −16.5%, p < 0.001 | −16.5%, p = 0.105 | 12.7%, p < 0.001 |
| | IL-37b (10) | −36.9%, p = 0.002 | −5.8%, p = 0.094 | −9.5%, p = 0.286 | 19.7%, p < 0.001 |
| | IL-37b (100) | −35.6%, p = 0.003 | −16.5%, p = 0.038 | −3.9%, p = 0.510 | 15.0%, p < 0.001 |
| | IL-38 (1) | −21.3%, p = 0.004 | −28.8%, p < 0.001 | 4.1%, p = 0.807 | 22.1%, p = 0.166 |
| | IL-38 (10) | −16.5%, p = 0.012 | −31.6%, p < 0.001 | 24.6%, p = 0.215 | 19.7%, p = 0.188 |
| | IL-38 (100) | −24.4%, p = 0.025 | −30.2%, p < 0.001 | 29.7%, p = 0.107 | 55.3%, p = 0.003 |
| | IL-37b (10) × | 12.7%, p = 0.409 | | 43.3%, p = 0.021 | |
| | IL-38 (10) | | | | |
| TNFα | IL-37b (1) | −18.1%, p = 0.018 | −9.5%, p = 0.627 | −56.0%, p = 0.170 | −25.9%, p = 0.399 |
| | IL-37b (10) | −28.8%, p = 0.020 | 17.4%, p = 0.431 | −60.1%, p = 0.109 | 0.0%, p = 1.000 |
| | IL-37b (100) | −28.8%, p = 0.020 | 0.0%, p = 1.000 | 45.1%, p = 0.020 | 35.0%, p = 0.145 |
| | IL-38 1 | −36.9%, p = 0.035 | −49.3%, p = 0.184 | 46.2%, p = 0.173 | 55.3%, p = 0.411 |
| | IL-38 10 | −44.0%, p = 0.116 | −40.5%, p = 0.311 | 49.2%, p = 0,123 | 166.4%, p = 0.105 |
| | IL-38 100 | −34.3%, p = 0.232 | −13.1%, p = 0.581 | 85.9%, p = 0.026 | 252.5%, p = 0.017 |
| | IL-37b (10) × | 19.7%, p = 0.445 | | 61.6%, p = 0.002 | |
| | IL-38 (10) | | | | |
| IL-10 | IL-37b (1) | −9.5%, p = 0.114 | −14.8%, p < 0.001 | −5.8%, p = 0.094 | 4.1%, p = 0.510 |
| | IL-37b (10) | −9.5%, p = 0.012 | 10.5%, p = 0.012 | −5.8%, p = 0.370 | 6.2%, p = 0.442 |
| | IL-37b (100) | −22.9%, p < 0.001 | −24.4%, p < 0.001 | −25.9%, p < 0.001 | −5.8%, p = 0.370 |
| | IL-38 (1) | −14.8%, p = 0.040 | 6.2%, p = 0.006 | 17.4%, p = 0.244 | 22.1%, p = 0.033 |
| | IL-38 (10) | −3.9%, p = 0.264 | 8.3%, p = 0.178 | 15.0%, p = 0.224 | 32.3%, p < 0.001 |
| | IL-38 (100) | −30.2%, p < 0.001 | −28.8%, p < 0.001 | 15.0%, p = 0.275 | 22.1%, p = 0.033 |
| | IL-37b (10) × | 35.0%, p = 0.021 | | 43.3%, p < 0.001 | |
| | IL-38 (10) | | | | |

The percentages represent the average effect of the plantakines treatments compared with the positive controls at corresponding concentrations. Calculated p-values are also displayed.
IM—Inflammatory Marker.

### DISCUSSION

In the present study we engineered the tobacco plant plastome to produce green bioreactors capable of manufacturing profuse amounts of two functional recombinant human cytokines, IL-37b and IL-38, mainly known as anti-inflammatory modulators of immune responses (Cavalli and Dinarello, 2018) (Xie et al., 2019). To our best knowledge, this is the first report describing such a prolific expression and production of both recombinant human cytokines in their active forms in plants, a previous successful attempt to produce IL-37b in tobacco via nuclear genome transformation reported much lower yields, while there are no reports on IL-38 production in plants hitherto (Alqazlan et al., 2019). Very interesting is the fact 10 that the penultimate amino acid (the second amino acid in the peptide chain after the initiating Methionine) of the IL-38 peptide is Cysteine, which was underlined, along with Histidine, as the strongest instability-conferring penultimate amino acid for protein expression and accumulation in plastids, leading the researchers to propose existence of an N-terminus-dependent protein degradation pathway in plastids (Apel et al., 2010). Our bioreactor lines produced IL-38 peptide with the penultimate Cysteine at 8%-10% of the TSP in the leaf tissue, contradicting the proposed model. Further, the same transformation construct expressing an IL-38 peptide variant with an added penultimate Serine, which was reported as a stabilizing penultimate amino acid, reached similar levels of IL-38 accumulation (data not shown), suggesting that the proposed N-terminus-dependent protein degradation pathway in chloroplasts is either limited in its processing capabilities, or involves additional unknown regulatory factors that specifically direct degradation of select proteins (Apel et al., 2010).

The accumulated dimerized (and multimerized) forms of the plantakines IL-37b and IL-38 in the crude leaf extracts and in purified samples constituted-50% of the entire recombinant protein yields, contrasting the results of successful recombinant production studies of IL-37b and IL-38 proteins in bacteria, which never reported dimerization of the purified cytokines in their SDS-PAGE analyses, even when the purified IL-37b was concentrated by ultrafiltration (Gu et al., 2015) (Yuan et al., 2016) (Hu et al., 2015). This observation suggests that the chloroplast stroma compartment, accumulating the synthesized recombinant proteins, provides a beneficial milieu of internal conditions/chaperones/scaffolds assisting the folding of these cytokines and promoting their further dimerization and multimerization. It is also reasonable to assume the remarkable stability of those dimer/multimeric forms of the cytokines in plastids, since the highest levels of accumulation were observed in older leaves. Bacteria-produced IL-37b was shown to form dimers at nanomolar concentrations and tetramers at higher concentrations, which greatly diminished its bioactivity, suggesting a mechanism of activity regulation through monomer/dimer equilibrium and leading to an engineered monomeric IL-37b variants with much stronger biological activity (Ellisdon et al., 2017) (Eisenmesser et al., 2019). Those monomeric variants, however, along with the natural mature recombinant IL-37b peptide showed appearance of minor bands that corresponded to the dimer size in SDS-PAGE analyses and further investigation of these protein structures is needed (Ellisdon et al., 2017). Also, intriguing is the question whether the formation of dimers/multimers contributes to the overall stability of those cytokines against proteolysis, thus enabling the proposed mechanism of self-regulation and in situ preservation in a stable inactive form in the intercellular space, where the local IL-37b concentration reached the dimerization constant values (Ellisdon et al., 2017). A mechanism of bioactivity regulation through the dimer formation, similar to that of IL-37b, was proposed for IL-38 in a recent review (Xie et al., 2019).

Bodily inflammatory processes are a part of innate immune responses, promoted by pro-inflammatory cytokines released from the cells of the immune system as a reaction to the presence of an inflammatory agent or stimuli. Secreted levels of 11 well-characterized inflammatory cytokines, namely GM-SCF, IFNγ, TNFα, IL-1α, IL-1β, IL-6, IL-8, IL-22, IL 12, IL-17 and IL-10 were monitored in our experiments with freshly isolated PBMCs subjected to treatments with two different IAs in combination with two plant-produced anti-inflammatory cytokines IL-37b and IL-38 at different concentrations. This experimental setup allowed for focusing on the biological effects exerted by the plantakines IL-37b and IL-38 via a direct comparison of the levels of pro-inflammatory cytokines secreted from IAs-stimulated PBMCs with or without the plantakines treatments at corresponding concentrations (Tables VI, VII). Although PHA and LPS bind to completely different sets of receptors, both IAs seem to trigger varying signal transduction cascades, particularly leading to the activation of NF-κB, which plays an essential role in regulating the expression of genes linked to innate immunity and inflammatory responses (Qureshi et al., 1999) (Liu et al., 2017). Seven out of 11 inflammatory cytokines monitored in our study displayed significantly higher secretion from PBMCs stimulated with PHA rather than LPS, IL-17 secretion varied more than 30-fold (FIG. 1). Remarkably, our findings strongly corroborate a recent report stating that after PHA-stimulation human PBMCs secreted significantly higher levels of inflammatory cytokines, compared to stimulation with LPS, which apparently failed to notably induce IL-17, TNFα and IL-12 (Lin et al., 2021).

The modulatory effects exerted on levels of the inflammatory markers secreted from IA-stimulated PBMCs confirmed the biological activity of plant-produced IL-37b and IL-38. Statistically significant modulations occurred in both LPS- and PHA-stimulated PBMCs due to treatments with the plantakines (Table VI). For quenching inflammation, treatments with plantakines appeared to be more effective in LPS-stimulated PBMCs, where 85% of all treatments resulted in attenuations of the levels of secreted inflammatory markers, while only 30% of treatments attenuated inflammatory markers in PHA-stimulated PBMCs, implying varying efficacy and specificity of the anti-inflammatory action under different stimuli. Attenuated, rather than increased secretion of inflammatory cytokines occurred 3.5 and 1.7 times more frequently in treatments with IL-37b and IL-38, respectively, in accord with the proposed role for IL-37b as a primary and fundamental inhibitor of inflammation (Cavalli and Dinarello, 2018) (Nold et al., 2010). Treatments combining both plantakines, however, resulted in increased secretion of inflammatory markers under either IA; also, with higher concentrations of either IA applied for PBMCs stimulation, treatments with plantakines suppressive of secretion of inflammatory cytokines became scarcer, while more treatments caused increased inflammatory secretion (Table VI).

Levels of the inflammatory markers secreted from the stimulated PBMCs displayed distinct and varying patterns of modulation following application of treatments with the plant-produced IL-37b and IL-38 (FIG. 24; Table VII). The number of treatments that triggered an increased, rather than attenuated secretion, was higher only in 2 among the 11 inflammatory cytokines monitored, namely GM-CSF and IL-12, indicating general anti-inflammatory effects exerted by the treatments with plantakines in IA-stimulated PBMCs. Notable were the differences in the magnitude and the scope of the modulation, reflected in the outcomes of the treatments being either an attenuation or an increase of the inflammatory secretion levels: an attenuation of the secretion was the outcome of 74 treatments, averaging-28% level reduction, while increases in secreted levels of inflammatory cytokines, observed in 44 treatments, displayed 79% on average. Among the 11 cytokines monitored, only the levels of IFNγ exhibited consistent attenuation from treatments with either plantakine in PBMCs stimulated with either IA, displaying also the strongest attenuation observed in our experiments (−63.9%, p<0.001), exerted by application of 1 ng/ml IL-38 in PBMCs stimulated with 150 μg/mL LPS. In contrast, GM-CSF levels were 3 times more frequently increased, rather than attenuated by treatments with the plantakines, with the strongest increase reaching 380.5%, p<0.001, upon application of 100 ng/ml IL-38 in PBMCs stimulated with 5 μg/mL PHA (Table VII). Strikingly, the plant-produced IL-37b and IL-38 both exerted dose-dependent regulation of GM-CSF secreted levels, bringing about attenuation at low concentrations, while causing increases at high concentrations. Although both IL-37b and IL-38 are generally characterized as anti-inflammatory cytokines active in quenching inflammation (van de Veerdonk et al., 2018) (Han et al., 2020), studies have reported that recombinant unprocessed IL-38 could increase inflammatory cytokine IL-6 production in human macrophages in response to LPS or IL-1β stimuli (van de Veerdonk et al., 2012) (Mora et al., 2016). In addition, IL-37b was reported to increase TNFα production in higher concentrations and Candida-induced IL-17 production was reportedly blocked by low concentrations of IL-38, while higher doses of IL-38 induced more IL-17 production, a pattern which resembled IL-37b bioactivity (Nold-Petry et al., 2015) (van de Veerdonk et al., 2012).

Both IFNγ and GM-CSF are crucial cytokines for activation/differentiation of myeloid cell populations (Ivashkiv, 2018) (Hamilton, 2019) and their nuanced regulation by the plant-produced IL-37b and IL-38 may serve as a primer for future studies to discern novel patterns in PBMCs inflammatory responses. Interesting also to note that statistically significant attenuation of IL-6 and IL-8, two profound inflammation markers monitored in our study (Tanaka et al., 2014) (Baggiolini and Clark-Lcwisb, 1992) was only detected upon applications of low concentrations of the plantakines, aligned with the anti-inflammatory functions expected from IL-37b and IL-38.

### Conclusions

A plastome-engineered, low-alkaloid tobacco bioreactor lines for cost-efficient and prolific production of two functional human cytokines with profound anti-inflammatory properties, IL-37b and IL-38, which are underlined as prospective therapeutic agents, have been developed. It has been demonstrated that the plantakines exerted significant modulation of levels of secreted cytokines involved in inflammatory responses monitored in IA-stimulated PBMCs, indicating a dose-dependent mode of action and general attenuation of several secreted inflammation markers. Enhancement of several pro-inflammatory cytokines, associated with higher concentrations of the plantakines applied in treatments was also observed, revealing novel patterns of inflammation regulation by IL-37b and IL-38. Different magnitude of responses from PBMCs were seen in levels of secreted cytokines elicited by different IAs, where PHA elicited stronger response than LPS in levels of most secreted cytokines monitored. Cumulatively, these results demonstrate feasibility of producing functional human recombinant cytokines in plants and further promote the accelerated adoption of plant-based manufacturing of various recombinant proteins by biotechnology industries.

### Methods

### Plastome Engineering

Plastome-engineered bioreactor lines expressing recombinant human IL-37b and IL-38 (UniProt identifiers Q9NZH6 and Q8WWZ1, respectively) in their mature forms (V46-D218 for IL-37b, C2-W152 for IL-38), each bearing a C-terminal hepta-HIS-tag, were produced by Igor Kolotilin for Solar Grants Biotechnology Inc.

### Recombinant Protein Extraction and Purification

Total soluble proteins from fresh leaf tissue of the bioreactor lines were extracted as described (Kolotilin et al., 2013). In short, flash-frozen leaf tissue was milled into powder with pestle and mortar and then 5 volumes of the extraction buffer (1λPBS, pH=7.4) was added, complemented with 10 μg/mL leupeptin 2 mM PMSF and 2% PVPP. After filtration through Whatman #1 paper, the extract was centrifuged twice for 15 minutes at 13,000λg in 4° C. The recombinant proteins were purified from the cleared extract utilizing the C-terminal His-tag and the immobilized metal ion-affinity chromatography (Cytiva Life Sciences™ His SpinTrap™, Cat. No. 28932171), dialysed against 1λPBS, pH=7.4 and filtered through 0.22 μm (EMD Millipore, Cat. No. SLGV004SL) to obtain sterile solutions.

### SDS-PAGE and Western Blots

SDS-PAGE and Western Blots experiments were performed as described previously (Kolotilin et al., 2013). Anti-His-tag antibodies (GenScript, Cat. No. A00186-100), as well as the monoclonal antibodies against human IL-37b and IL-38 (MyBioSource Inc., Cat. No. MBS 7600509 and R&D Systems, Cat. No. DY9110-05, respectively) were used to detect the blotted proteins according to manufacturers' recommendations; bacteria-produced recombinant human IL-37b (R&D Systems, Cat. No. 7585-IL-025) and IL-38 MyBioSource, Cat. No. MBS635478) were used as the positive controls. Western blots membranes were visualized using the enhanced chemiluminescence (ECL) detection kit (GE Healthcare, Cat. No. RPN2232) and imaged with the DNR Bio-Imaging System MicroChemi (RANCOM A/S, Birkerød, Denmark). Densitometry was performed using the TotalLab TL 100 software (Nonlinear Dynamics, Durham, NC).

### ELISA Experiments

ELISA experiments with the plantakines IL-37b (Invitrogen™, Cat. No. LS885210322) and IL-38 (R&D Systems Inc., Cat. No. DY9110-05) were performed according to manufacturers' recommendations. BioTek Instruments (VT, USA) Epoch Microplate Spectrophotometer was used to acquire numerical ELISA data.

Experiments with PBMCs—General Design and Multiplex Cytokine Analysis

After isolation, the PBMCs from each donor separately were counted, plated in equal numbers per well and stimulated for 24 h with the applied treatments. Control wells on the plate contained media only (as basal level controls), and LPS and PHA (as positive controls at their corresponding concentrations). After the 24 h treatments stimulation the PBMCs' culture supernatants were used in the multiplex Luminex platform-assisted analysis of the concentrations of the 11 secreted pro-inflammatory cytokines.

The whole blood from 5 random human donors was collected in ACD Vacutainer tubes and immediately processed for isolation of PBMCs by gradient density centrifugation using Lympholyte. The freshly isolated PBMCs from each donor separately were cultured in a volume of 200 μL at a concentration of 1.25×10⁶ cells/mL in 96-well plates (˜250,000 cells/well) in an incubator set at 37° C. 5% CO2 and >80% humidity. PBMCs were treated with one of two IAs at 2 concentrations each (LPS at 150 and 300 μg/mL; PHA at 5 and 10 μg/mL) in combination with two test items (plant-produced IL-37b or IL-38) at three concentrations each (1, 10 and 100 ng/ml), based on the monomeric form amounts estimated with densitometry (ImageJ). Each treatment was tested in triplicates. Control wells contained media only (negative control, basal level of detection) and the examined IAs at both tested concentrations as the positive controls. The PBMCs were incubated for 24 hours after the stimulation for analysis of the levels of the secreted cytokines that were determined in the culture supernatant using a multiplex immunoassay (MAGPix®, Luminex), analytes were selected from the Milliplex panel HCYTOMAG-60K, sensitivity range of 3.2 to 10,000 μg/mL. All parameters of the Milliplex panel cytokines analysis using Luminex platform were validated (Millipore-Sigma).

### Statistical Analysis

Non-linear logarithm transformation was performed to address non-normality of distribution of Luminex-derived numeric data. Generalized Estimating Equation (GEE) method was used in the analysis of nested (correlated) structure of the data. Secreted inflammatory cytokine values were used as dependent variables and test item (plantakines) with dosage as independent predictors (factors). Separate analyses were conducted for each of the 11 monitored cytokines and each inflammatory agent/concentration combination. Analysis was performed using SPSS software version 27 using the level of significance 0.05 (p-values <0.05 are reported as statistically significant).

### REFERENCES

Adem, M., Beyene, D., and Feyissa, T. (2017). Recent achievements obtained by chloroplast transformation. Plant Methods 13, 30.
Ahmad, N., Michoux, F., Lössl, A. G., and Nixon, P. J. (2016). Challenges and perspectives in commercializing plastid transformation technology. Journal of Experimental Botany 67, 5945-5960.
Alam, A., Jiang, L., Kittleson, G. A., Steadman, K. D., Nandi, S., Fuqua, J. L., Palmer, K. E., Tuse, D., and McDonald, K. A. (2018). Technoeconomic Modeling of Plant-Based Griffithsin Manufacturing. Frontiers in Bioengineering and Biotechnology 6, 102.
Alqazlan, N., Diao, H., Jevnikar, A. M., and Ma, S. (2019). Production of functional human interleukin 37 using plants. Plant Cell Reports 38, 391-401.
Apel, W., Schulze, W. X., and Bock, R. (2010). Identification of protein stability determinants in chloroplasts. Plant Journal 63, 636-650.
Baggiolini, M., and Clark-Lcwisb, I. (1992), number 1, 97-101 FEWI 1242 0 1992 Federation of European Biochemical Societies.
Buyel, J. F., Twyman, R. M., and Fischer, R. (2017). Very-large-scale production of antibodies in plants: The biologization of manufacturing. Biotechnology Advances 35, 458-465.
Cavalli, G., and Dinarello, C. A. (2018). Suppression of inflammation and acquired immunity by IL-37. Immunological Reviews 281, 179-190.
Daniell, H., Jin, S., Zhu, X. G., Gitzendanner, M. A., Soltis, D. E., and Soltis, P. S. (2021). Green giant-a tiny chloroplast genome with mighty power to produce high-value proteins: history and phylogeny. Plant Biotechnology Journal 19, 430-447.
Dinarello, C. A., Nold-Petry, C., Nold, M., Fujita, M., Li, S., Kim, S., and Bufler, P. (2016). Suppression of innate inflammation and immunity by interleukin-37. Eur. J. Immunol 46, 1067-1081.
Eisenmesser, E. Z., Gottschlich, A., Redzic, J. S., Paukovich, N., Nix, J. C., Azam, T., Zhang, L., Zhao, R., Kieft, J. S., The, E., et al. (2019). Interleukin-37 monomer is the active form for reducing innate immunity. Proceedings of the National Academy of Sciences of the United States of America 116, 5514-5522.
Ellisdon, A. M., Nold-Petry, C. A., D'andrea, L., Cho. S. X., Lao, J. C., Rudloff, I., Ngo, D., Lo, C. Y., Soares Da Costa, T. P., Perugini, M. A., et al. (2017). Homodimerization attenuates the anti-inflammatory activity of interleukin-37.
Ferreira De Mello, V. D., Kolehmanien, M., Schwab, U., Pulkkinen, L., and Uusitupa, M. (2012). Gene expression of peripheral blood mononuclear cells as a tool in dietary intervention studies: What do we know so far? Mol. Nutr. Food Res 56, 1160-1172.
Fox, J. L. (2012). First plant-made biologic approved.
Gengenbach, B. B., Keil, L. L., Opdensteinen, P., Müschen, C. R., Melmer, G., Lentzen, H., Bührmann, J., and Buyel, J. F. (2019). Comparison of microbial and transient expression (tobacco plants and plant-cell packs) for the production and purification of the anticancer mistletoe lectin viscumin. Biotechnology and Bioengineering 116, 2236-2249.
Gu, J., Gao, X., Pan, X., Peng, X., Li, Y., and Li, M. (2015). High-level expression and one-step purification of a soluble recombinant human interleukin-37b in Escherichia coli. Protein Expression and Purification 108, 18-22.
Hamilton, J. A. (2019). Cytokines Focus GM-CSF in inflammation.
Han, Y., Huard, A., Mora, J., da Silva, P., Brune, B., and Weigert, A. (2020). IL-36 family cytokines in protective versus destructive inflammation. Cellular Signalling 75.
Hartmann, J., Schüβler-Lenz, M., Bondanza, A., and Buchholz, C. J. Clinical development of CAR T cells-challenges and opportunities in translating innovative treatment concepts.
Hu, Z., Chen, Z., Huang, N., Teng, X., Zhang, J., Wang, Z., Wei, X., Qin, K., Liu, X., Wu, X., et al. (2015). Expression, purification of IL-38 in Escherichia coli and production of polyclonal antibodies. Protein Expression and Purification 107, 76-82.
Huebbers, J. W., and Buyel, J. F. (2021). On the verge of the market-Plant factories for the automated and standardized production of biopharmaceuticals. Biotechnology Advances 46.
Ivashkiv, L. B. (2018). IFNγ: signalling, epigenetics and roles in immunity, metabolism, disease and cancer immunotherapy HHS Public Access. Nat Rev Immunol 18, 545-558.
Kleiveland, C., and Kleiveland, C. (2015). Peripheral blood mononuclear cells. In The Impact of Food Bioactives on Health: In Vitro and Ex Vivo Models, (Springer International Publishing), pp. 161-167.
Kolotilin, I., Kaldis, A., Pereira, E. O., Laberge, S., and Menassa, R. (2013). Optimization of transplastomic production of hemicellulases in tobacco: effects of expression cassette configuration and tobacco cultivar used as production platform on recombinant protein yields. Biotechnology for Biofuels 6, 65.
Lin, Z., Huang, Y., Jiang, H., Zhang, D., Yang, Y., Geng, X., and Li, B. (2021). Functional differences and similarities in activated peripheral blood mononuclear cells by lipopolysaccharide or phytohemagglutinin stimulation between human and cynomolgus monkeys. Annals of Translational Medicine 9, 257-257.
Liu, T., Zhang, L., Joo, D., and Sun, S.-C. (2017). NF-κB signaling in inflammation.
Maliga, P., and Bock, R. (2011). Plastid biotechnology: Food, fuel, and medicine for the 21st century. Plant Physiology 155, 1501-1510.
Martínez-Rodriguez, N. L., Tavárez, S., and González-Sánchez, Z. I. (2019). In vitro toxicity assessment of zinc and nickel ferrite nanoparticles in human erythrocytes and peripheral blood mononuclear cell. Toxicology in Vitro 57, 54-61.
McNulty, M. J., Gleba, Y., Tuse, D., Hahn-Lobmann, S., Giritch, A., Nandi, S., and McDonald, K. A. (2020). Techno-economic analysis of a plant-based platform for manufacturing antimicrobial proteins for food safety. Biotechnology Progress 36.
Mora, J., Schlemmer, A., Wittig, I., Richter, F., Putyrski, M., Frank, A. C., Han, Y., Jung, M., Ernst, A., Weigert, A., et al. (2016). Interleukin-38 is released from apoptotic cells to limit inflammatory macrophage responses. Journal of Molecular Cell Biology 8, 426-438.
Nold, M. F., Nold-Petry, C. A., Zepp, J. A., Palmer, B. E., Bufler, P., and Dinarello, C. A. (2010). IL-37 is a fundamental inhibitor of innate immunity. Nature Immunology 11, 1014-1022.
Nold-Petry, C. A., Lo, C. Y., Rudloff, I., Elgass, K. D., Li, S., Gantier, M. P., Lotz-Havla, A. S., Gersting, S. W., Cho, S. X., Lao, J. C., et al. (2015). IL-37 requires the receptors IL-18Ra and IL-1R8 (SIGIRR) to carry out its multifaceted anti-inflammatory program upon innate signal transduction. Nature Immunology 16, 354-365.
Oda, S., Uchida, Y., Aleo, M. D., Koza-Taylor, P. H., Matsui, Y., Hizue, M., Marroquin. L. D., Whritenour, J., Uchida, E., and Yokoi, T. (2021). An in vitro coculture system of human peripheral blood mononuclear cells with hepatocellular carcinoma-derived cells for predicting drug-induced liver injury. Archives of Toxicology 95, 149-168.
Palomo, J., Dietrich, D., Martin, P., Palmer, G., and Gabay, C. (2015). The interleukin (IL)-1 cytokine family-Balance between agonists and antagonists in inflammatory diseases. Cytokine 76, 25-37.
Qureshi, S. T., Larivière, L., Leveque, G., Clermont, S., Moore, K. J., Gros, P., and Malo, D. (1999). Endotoxin-tolerant mice have mutations in toll-like receptor 4 (Tlr4). Journal of Experimental Medicine 189, 615-625.
Ramírez-Alanis, I. A., Renaud, J. B., García-Lara, S., Menassa, R., and Cardineau, G. A. (2018). Transient co-expression with three O-glycosylation enzymes allows production of GalNAc-O-glycosylated Granulocyte-Colony Stimulating Factor in N. benthamiana. Plant Methods 14, 1-14.
Schillberg, S., and Finnern, R. (2021a). Plant molecular farming for the production of valuable proteins-Critical evaluation of achievements and future challenges. Journal of Plant Physiology 258-259.
Schillberg, S., and Finnern, R. (2021b). Plant molecular farming for the production of valuable proteins-Critical evaluation of achievements and future challenges. Journal of Plant Physiology 258-259.
Shanmugaraj, B., Bulaon, C. J. I., and Phoolcharoen, W. (2020). Plant molecular farming: A viable platform for recombinant biopharmaceutical production. Plants 9, 1-19.
Tanaka, T., Narazaki, M., and Kishimoto, T. (2014). Cite this article as. Cold Spring Harb Perspect Biol 6.
Tsekoa, T. L., Singh, A. A., and Buthelezi, S. G. (2020). Molecular farming for therapies and vaccines in Africa. Current Opinion in Biotechnology 61, 89-95.
van de Veerdonk, F. L., Stoeckman, A. K., Wu, G., Boeckermann, A. N., Azam, T., Netea, M. G., Joosten, L. A. B., van der Meer, J. W. M., Hao, R., Kalabokis, V., et al. (2012). IL-38 binds to the IL-36 receptor and has biological effects on immune cells similar to IL-36 receptor antagonist. Proceedings of the National Academy of Sciences of the United States of America 109, 3001-3005.
van de Veerdonk, F. L., de Graaf, D. M., Joosten, L. A., and Dinarello, C. A. (2018). Biology of IL-38 and its role in disease. Immunological Reviews 281, 191-196.
Ward, B. J., Makarkov, A., Séguin, A., Pillet, S., Trépanier, S., Dhaliwall, J., Libman, M. D., Vesikari, T., and Landry, N. (2020). Efficacy, immunogenicity, and safety of a plant-derived, quadrivalent, virus-like particle influenza vaccine in adults (18-64 years) and older adults (≥65 years): two multicentre, randomised phase 3 trials. The Lancet 396, 1491-1503.
Wettstein. J., Matteson, E. L., Strausbauch, S. E. G., Crowson, C. S., Themeau, T. M., Peter, J. M., Davis, K. L., and Knutson, M. A. (2019). Cells Cytokine Responsiveness of Peripheral Blood Immune-Mediated Disorders Based on Analysis of Complex Biomarkers for Human. J Immunol References 184, 7297-7304.
Xie, L., Huang, Z., Li. H., Liu, X., Zheng, S., and Su, W. (2019). IL-38: A new player in inflammatory autoimmune disorders. Biomolecules 9.
Xu, W. D., and Huang, A. F. (2018). Role of interleukin-38 in chronic inflammatory diseases: A comprehensive review. Frontiers in Immunology 9.
Xu, S., Gavin, J., Jiang, R., and Chen, H. (2017). Bioreactor productivity and media cost comparison for different intensified cell culture processes. Biotechnology Progress 33, 867-878.
Yuan, X. L., Li, Y., Pan, X. H., Zhou, M., Gao, Q. Y., and Li, M. C. (2016). Production of recombinant human interleukin-38 and its inhibitory effect on the expression of proinflammatory cytokines in THP-1 cells. Molecular Biology 50, 405-411.

### Example 12. Induction of IL-6 in Human Cells by SARS-COV-2-Derived Peptide is Attenuated by Recombinant Human Anti-Inflammatory Cytokines Made in Planta

### SUMMARY

Development of efficient therapies for COVID-19 is the focus of intense research. The cytokine release syndrome was underlined as a culprit for severe outcomes in COVID-19 patients. Interleukin-6 (IL-6) plays a crucial role in human immune responses and elevated IL-6 plasma levels have been associated with the exacerbated COVID-19 pathology. Since non-structural protein 10 (NSP10) of SARS-COV-2 has been implicated in the induction of IL-6, Peptide (P) 1, containing sequences corresponding to amino acids 68-96 of NSP10 were designed, and examined for its effect on cultured human cells. Treatment with P1 strongly increased IL-6 secretion by the lung cancer cell line NCI-H1792 and the breast cancer cell line MDA-MB-231 and revealed profound cytotoxic activity on Caco-2 colorectal adenocarcinoma cells. Treatment with P2, harbouring a mutation in the zinc knuckle motif of NSP10, caused no IL-6 induction and no cytotoxicity. Pre-treatment with plant-produced human anti-inflammatory cytokines IL-37b and IL-38 effectively mitigated the induction of IL-6 secretion. Our results suggest a role for the zinc knuckle motif of NSP10 in the onset of increased IL-6 plasma levels of COVID-19 patients and for IL-37b and IL-38 as therapeutics aimed at attenuating the cytokine release syndrome.

### INTRODUCTION

The Severe Acute Respiratory Syndrome Coronavirus 2 (SARS-COV-2) is a highly transmissible pathogenic coronavirus. It is responsible for the coronavirus disease 2019 (COVID-19) pandemic, resulting in threats to human health worldwide (WHO, 2020). The pathogenesis of the SARS-COV-2 in humans varies in manifestation from mild cold-like symptoms to severe respiratory failure (Hu et al., 2020). Upon binding to epithelial cells in the respiratory tract, the virus starts to replicate and eventually migrates down to the alveolar epithelial cells in the lungs, where its replication increases significantly, inducing strong immune responses (Pedersen and Ho, 2020) (Tang et al., 2020). This increase in replication is accompanied by the “cytokine storm”, which brings about respiratory distress syndrome and respiratory failure. The resulting over-exuberant immune response is considered the main cause of death in COVID-19 patients (Tian et al., 2020). With the newly emerging variants of the SARS-COV-2 virus displaying resistance towards neutralizing antibodies, hence threatening the efficacy of vaccines (Liu et al., 2020b), alternative therapeutic approaches are more needed than ever.

The likelihood of a more severe manifestation of the viral infection in COVID-19 patients appears to correlate with the plasma levels of certain cytokines (Pedersen and Ho, 2020). Of a particular interest is the pleiotropic cytokine Interleukin-6 (IL-6) and its role in SARS-COV-2 infections, and viral infections in general (Velazquez-Salinas et al., 2019). It has been well-established that the levels of IL-6 increase during the acute phase of infection with vesicular stomatitis virus (VSV) and this increase was associated with higher virulence in pigs (Velazquez-Salinas et al., 2018). Hospitalized patients infected with Andes virus (ANDV) displayed significantly elevated levels of IL-6, and the magnitude of the increase correlated with the severity of symptoms (Angulo et al., 2017). In vitro studies in cells revealed that a recombinant Spike(S) protein of SARS-COV strongly induced production of IL-6 in murine macrophages (Wang et al., 2007). IL-6 knockout mice showed high mortality when challenged with sub-lethal doses of H1N1 influenza virus, while WT mice recovered from the infection (Dienz et al., 2012). Importantly, the serum levels of IL-6 are markedly increased during the SARS-COV-2 infection and IL-6 was proposed to be a reliable predictive biomarker of the severity of the COVID-19 in hospitalized patients (Pedersen and Ho, 2020). (Conti et al., 2020a) (Chen et al., 2020; Liu et al., 2020a). Produced by various cell types, IL-6 induces a signaling cascade involving the JAK/STAT3 pathway to regulate transcription of a multitude of genes involved in cellular signaling and regulation of gene expression (Mauer et al., 2015; Wang et al., 2013). With its crucial involvement in both pro- and anti-inflammatory processes, IL-6 was attributed a central role in the regulation of immune responses (Scheller et al., 2011).

Certain proteins of the SARS-COV-2 virus have been implicated in inducing the increase of IL-6 including the S protein, Nucleocapsid (N) protein, and Non-Structural Protein 10 (NSP10) (Gordon et al., 2020). The effects that some of these proteins exerted on IL-6 have been known since their homologous counterparts were discovered in SARS-COV (Wang et al., 2007). The NSP10 protein in coronaviruses appears to constitute a cofactor in the methyltransferase complexes it forms with NSP14 and NSP16 (Wang et al., 2015). The crystal structure of the NSP10/NSP16 complex of SARS-COV-2 was solved in a recent study (Krafcikova et al., 2020). Previously, peptides derived from the NSP10 sequence involved in the interaction with NSP16 of Murine Hepatitis Virus (MHV) were reported to be successful inhibitors of NSP16 2′-O-Methyltransferase activity in vitro, as well as rescuing mice infected with MHV (Wang et al., 2015). Peptides derived from the region of NSP10 that interacts with NSP16 of SARS-COV virus were demonstrated to inhibit the 2′-O-methyltransferase activity in cell-free biochemical assays (Ke et al., 2012). While the potential of NSP10-derived peptides of SARS-COV to inhibit 2′-O-methyltransferase has not been tested in cell culture, the full length NSP10 protein is known to induce increases in IL-6 levels (Gordon et al., 2020). Interestingly, the region of the NSP10 protein that forms the interaction surface with NSP16 contains a zinc “knuckle” motif. Such motifs are known to correlate with activation of IL-6. In osteoarthiritic mice, the suppression of the ZCCHC6 protein containing this domain correlated with a reduction in IL-6 expression (Ansari et al., 2019). On the other hand, expression of ZCCHC6 in osteoarthritis chondrocytes correlated with an increase in IL-6 (Akhtar et al., 2014).

Elucidating the molecular mechanisms underlining SARS-COV-2-induced pathology and searching for potential efficacious treatments to COVID-19 represent the urgent focus of the worldwide scientific community. The regulation of the bodily inflammatory responses is exerted by an intricate network of various types of mediator molecules produced by and exchanged between different cells of the immune system (Cronkite and Strutt, 2018). The IL-1 cytokine superfamily plays a crucial role in immune system homeostasis, various autoimmune pathologies, and autoinflammation (Mantovani et al., 2019). Two relatively recently discovered members of the IL-1 cytokine superfamily, IL-37 and IL-38, exhibit profound anti-inflammatory activities (Nold et al., 2010) (Lin et al., 2001). A plethora of scientific studies aimed at elucidating the biological roles of these cytokines demonstrated their pivotal action in both innate and adaptive immune responses, anti-tumor activity, and their essential involvement in mechanisms underlying diverse pathological conditions and autoimmune disorders, thus positioning those two cytokines as promising candidates for development as prospective therapeutic agents (Mei and Liu, 2019) (Allam et al., 2020) (Yang et al., 2019) (Ummarino, 2017) (Xu and Huang, 2018) (Xie et al., 2019). The use of both IL-37 and IL-38 has been proposed as a valid therapeutic approach looking to mitigate SARS-COV-2 infection-associated immunopathology and control the acute detrimental pulmonary inflammation seen in COVID-19 (Conti et al., 2020b). Recent clinical findings linked the elevated plasma levels of IL-37 as an early response in SARS-COV-2-infected patients with a positive clinical prognosis and earlier hospital discharge, whereas lower IL-37 early responses predicted severe illness. Higher blood IL-37 levels in those patients correlated with reduced IL-6 and IL-8 levels. Furthermore, ACE2-transgenic mice infected with SARS-COV-2 showed alleviation of lung tissue damage, when treated with recombinant IL-37 (Li et al., 2020a). Importantly, the use of Tocilizumab (a neutralizing antibody raised against human IL-6) has been reported to be safe in human trials, albeit with mixed results of efficacy, suggesting exploration of other therapeutic routes for IL-6 mitigation (Masiá et al., 2020).

In the current study, two peptides, P1 and P2, derived from the SARS-COV-2's NSP10 protein region that forms the interaction surface with NSP16, were employed. The sequence of P1 was completely homologous to NSP10's amino acids 68-96, while P2 contained the amino acid substitution, Histidine80 to Arginine (H80R), which was designed to disrupt the zinc knuckle motif. Both peptides were engineered with an N-terminal 14 amino acid sequence corresponding to the protein transduction domain of the HIV's Trans-Activator of Transcription (TAT) protein to allow penetration of the cell membrane. Based on previous studies demonstrating a role of full length NSP10 in stimulating the secretion of IL-6 lung epithelial A549 cells (Li et al . . . 2020b), it was hypothesized that treating cultured human cells with the designed peptides P1 and P2 could help elucidate the involvement of the zinc knuckle motif of the viral NSP10 in increased IL-6 secretion. Interestingly, upon application of the peptides onto human lung cells a profound induction of IL-6 secretion was observed in the case of P1, but not P2. We further hypothesized that the increases in IL-6 secretion could be mitigated by the use of recombinant anti-inflammatory cytokines, such as IL-3 7b and IL-38. We found that treatment with recombinant IL-37b and IL-38, produced in engineered tobacco plants, significantly reduced the levels of IL-6 induced by P1 in human lung cells, demonstrating their potential application as treatment against the cytokine storm caused by COVID-19.

### Results

### Design of NSP10-Derived Peptides

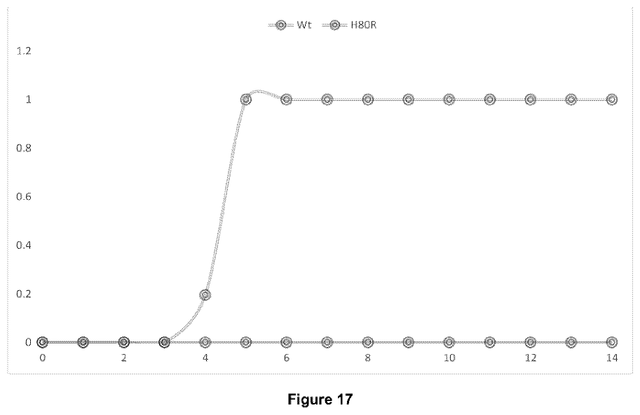

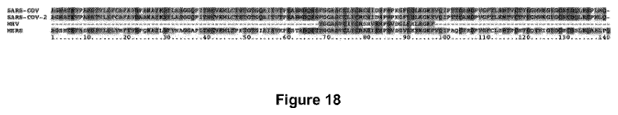

We designed two peptides (P1 and P2) derived from NSP10 from SARS-COV-2. P1 contains the CCHC Zinc finger motif of NSP10. As a control, P2 differed from P1 in only one amino acid at position 26 (Table VIII), replacing the histidine of the CCHC motif with an arginine (corresponding to substitution H80R in full length NSP10). Multi-Conformer Continuum Electrostatics (MCCE) calculations suggested that the binding of zinc to the coordination site is greatly affected (by over 90%) due to this mutation (FIG. 17). In keeping with previous work, both peptides were conjugated to a 14 amino acid long HIV TAT sequence to allow for cell membrane penetration. FIG. 18 shows the alignment of the sequences of NSP10 from SARS-CoV, SARS-COV-2, MHV and Middle East Respiratory Syndrome (MERS)-CoV. NSP10 sequences of SARS-COV and SARS-COV-2 are identical with respect to the region of interest, while sequences from MHV and MERS-COV share a Proline to Valine substitution, likely altering the conformation dramatically.

Table VIII. Amino acids sequences of Peptides P1 and P2 derived from the NSP10 protein of SARS-COV-2 involved in the interaction with NSP16. Italics indicate amino acids of HIV-Tat sequence required for membrane penetration. Bold and underlined letters indicate substitution of Histidine with Arginine at position 26, corresponding to amino acid residue 80 in full length NSP10 (H80R).

| Peptide | Sequence |
|---|---|
| P 1 | YGRKKRRQRRRGSGFGGASCCLYCRCHIDHPN |
| (SEQ ID NO. 40) | PKGFCDLKGKY |
| P 2 | YGRKKRRQRRRGSGFGGASCCLYCRCRIDHPN |
| (SEQ ID NO. 41) | PKGFCDLKGKY |

SARS-COV-2 NSP10-derived peptide (P1) induces secretion of IL-6 by human lung cells

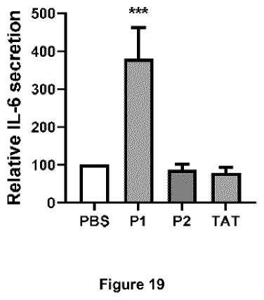

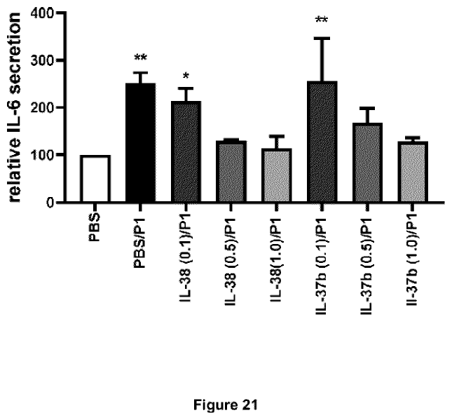

We first determined the effect of peptides P1 and P2 on the secretion of IL-6 in human cell lines. As shown in FIG. 19, incubation of the human NSCLC cell line NCI-H1792 with P1, but not P2 or a TAT sequence peptide only, resulted in a more than a 4-fold stimulation (P<0.001) of the intrinsic secretion of IL-6. Similar results were obtained with the human metastatic breast cancer cell line MDA-MB-231 (FIG. 21)

Plant-produced IL-37b and IL-38 effectively attenuate the levels of IL-6 induced by P1 We next determined the effect of plant-produced anti-inflammatory cytokines IL-37b and IL-38 on the P1-elicited stimulation of IL-6 secretion by H1792 cells. Pre-treatment with IL-38 resulted in pronounced attenuation (−60%; P<0.001) of IL-6 secretion triggered by P1 (FIG. 20). This attenuation effect, although less strong, was also observed when cells were pre-treated with IL-37b (−40%; P<0.05), or with a combination of IL-38 and IL-37b (−50%; P<0.05). Similar results were obtained using MDA-MB-231 cells. As shown in FIG. 21, the attenuating effect of IL-38 and IL-37b was dose-dependent.

### P1 and P2 Cytotoxic Activity

As the next step in the assessment of the biological action of peptides P1 and P2, their cytotoxicity profiles were determined, considering that NSP10 was shown to be implicated in multiple interactions with various host cell and viral proteins (Bouvet et al., 2014). Since Caco-2 cells are now routinely used in studies to examine potential drugs against SARS-COV-2 due to viral preference for replication (Cagno, 2020), the toxicity of P1 and P2 against Caco-2 cells was tested. The results showed that while P2 had no detectable toxicity at concentrations up to 200 μM. P1 displayed a Cytotoxic Concentration (CC50) of 11 μM.

### DISCUSSION

### Designed Peptides and Cellular Responses

The region of NSP10 corresponding to peptide P1 has previously been shown to inhibit complex formation between NSP10 and NSP16 from SARS-COV (Ke et al., 2012). In another study, NSP10-derived peptides from MHV were used to demonstrate reduction in viral pathogenesis in cell lines and in mice (Wang et al., 2015). In the present study, the P1 peptide based on the sequence of NSP10 from SARS-COV-2 (identical in NSP10 from SARS-COV) that is homologous to the MHV NSP10 sequence, has been designed.

As a part of the NSP 10 structure, the sequence of P1 contains a zinc knuckle motif consisting of a zinc ion coordinated by three cysteines and a histidine residue. Interestingly, only P1, but not P2, stimulated IL-6 production and caused cytotoxicity in these experiments. Peptide P2 differs from P1 in only one amino acid, replacing the histidine residue from the zinc finger motif by arginine. Our results therefore suggest that the zinc coordination site, which is greatly affected by the substitution of His by Arg in peptide P2, is needed for the induction of IL-6 expression. This could explain why P2 does not induce IL-6 in the same manner as P1. In a series of experiments to be reported elsewhere, it has been determined that a peptide corresponding to a different interface region of the NSP10/NSP16 complex did not induce IL-6 and indeed reduced replication of SARS-COV-2 in Caco-2 cells infected with the virus.

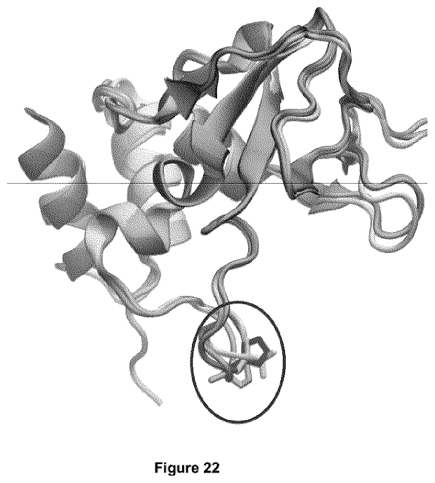

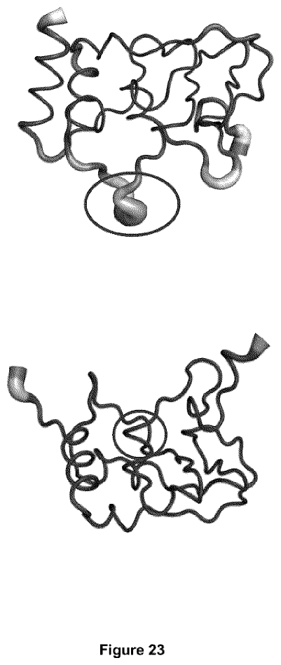

As seen in FIG. 22, the structure of MERS NSP10, which shares the Proline to Valine substitution with MHV, is slightly different from SARS-COV and SARS-COV-2 NSP10. While no experimental structure of the NSP10 protein of MHV exists, the MERS structure is available (PDB: 5YN5) as well as the SARS-COV-2 NSP10 structure (DPDB ID: 6W4H). We performed Normal Mode Analysis (NMA) using the DynaMut server on both structures to examine the areas of atomic fluctuations. The NMA results showed that MERS NSP10 has more deformation energies around the zinc knuckle, making the MERS NSP10 structure less stable around the Zinc finger (FIG. 23). Moreover, the residue corresponding to His80 in NSP10 from SARS-CoV, SARS-COV-2, and MERS is an arginine in MHV. Taken together, this could provide an explanation as to why MHV NSP10 does not elicit an increase in IL-6 once applied to cells and mice. An alternative explanation could be that the reaction of murine cells is different from human cells. An experiment in which the levels of IL-6 are measured in mice and Caco-2 cells due to exposure to MHV NSP10-derived peptides, as well as SARS-COV-2 NSP10-derived peptides would be beneficial to shed light on this inconsistency.

### Possible Mechanism of IL-6 Induction by the NSP10-Derived P1

Host-virus interactome derived from proteomics and co-immunoprecipitation assays have suggested that NSP10 inhibits the NF-B-repressing factor (NKRF) to facilitate interleukin-8 (IL-8) induction, and possibly IL-6 (Li et al., 2021). This could potentially contribute to interleukin-mediated chemotaxis of neutrophils and the over-exuberant host inflammatory response observed in COVID-19 patients (Li et al., 2021). The link between NF-B and IL-6 stimulation has been previously well-established. NF-B is known to regulate an IL-6 mRNA stabilizing protein, known as AT-rich interactive domain-containing protein 5a (Arid5a) (Nyati et al., 2017). Toll-Like Receptor 4 (TLR4) induces NF-B, which in turn activates Arid5a and subsequently induces IL-6 expression. While no previous studies examined whether NSP10 has a direct role in activating TLR4, the S protein of SARS-COV-2 has been linked to TLR4 (Brandão et al., 2020). There have been suggestions in the literature that SARS-COV-2 non-structural proteins affect Toll-Like Receptors in general. In fact, both SARS-COV and MERS-COV viruses are known to affect TLR4 and TLR3 (Totura et al., 2015). However, in the case of SARS-COV and MERS-COV, the mechanism appears to be protective against the viral infection.

Curbing P1-Induced IL-6 Expression with Plant-Produced Anti-Inflammatory Cytokines

Increased IL-6 expression in cells in response to P1 application could be curbed by treatment with recombinant, in planta-produced human cytokines IL-38 and IL-37b. It is reasonable to attribute the observed attenuation of IL-6 expression to the biological action of the cytokines, since treatment with the control protein GFP, bearing an identical His-tag, did not result in any significant attenuation. The anti-inflammatory action of both IL-38 and IL-37b was previously reported as dose-dependent, displaying bell-shaped concentration efficiency, with optimal concentrations ranging from 10 to 100 ng/ml (Nold-Petry et al., 2015; Van De Veerdonk et al., 2012). In addition, (Gu et al., 2015) showed that increasing concentrations of IL-37b (up to 500 ng/ml) applied in LPS-challenged THP-1 cells led to more significant inhibition of TNF and IL-1 expression. In this regard, the pronounced attenuation of the levels of IL-6, an inflammation-associated marker, observed in these experiments is in accord with previous reports characterizing the anti-inflammatory nature of IL-38 and IL-37b at these concentrations (Cavalli and Dinarello, 2018; Gu et al., 2015; Han et al., 2020).

Despite their binding to a completely different set of receptors, the inhibitory action of both IL-37b and IL-38 was shown on the intracellular signal transduction pathways of different STATs, p38MAPK, ERK1/2 and JNK, as well as NF-B signaling, outlining a degree of redundancy (Gao et al., 2021; Nold-Petry et al., 2015). IL-37 binds to IL-18R and recruits the IL-1R8 (also called SIGIRR or TIR8) to inhibit pro-inflammatory signalling (Nold-Petry et al., 2015). IL-37 also acts in the nucleus with Smad3 suppressing expression of inflammatory genes (Nold et al., 2010). IL-38 was first characterized as similar to IL-36Ra for its antagonist function on IL-36R, however, the mechanism of IL-38 signalling is not yet fully elucidated and appears to play a role in inflammation resolution (Van De Veerdonk et al., 2012). IL-38 has been shown to bind to three receptors: IL-36R, IL-1R1 and IL-1 receptor accessory protein-like 1 (IL-1RAPL1), thus exerting anti-inflammatory effects by competing with their agonistic ligands and inhibiting their signalling pathways (Mora et al., 2016) (Yuan et al., 2016).

IL-38 produced a stronger inhibitory effect on IL-6 levels in comparison with either the action of IL-37b, or application of a combination of both recombinant cytokines (FIG. 20). This observation is in agreement with their redundant inhibitory action on pro-inflammatory signalling leading to IL-6 expression, also indicating the absence of possible synergistic effects from the simultaneous application of IL-37b and IL-38.

### Conclusion

Our results demonstrated that a peptide derived from SARS-COV-2 NSP10 can cause a significant increase of IL-6 secretion in human adenocarcinoma lung cells and resulted in cytotoxicity in an intestinal epithelial cell line. Our results also indicate that it is the zinc knuckle motif of NSP10, preserved in P1, but not in P2, that is likely to elicit this IL-6 inflammation marker increase response, emphasized by the notion that this motif is also found in other proteins known to cause increases in IL-6 expression. Since the elevated levels of IL-6 are a predictive molecular marker for the cytokine storm that accompanies COVID-19 pathology, correlating with poor prognosis, these findings suggest that therapeutic targeting the NSP10-induced inflammation should be pursued. IL-37b and IL-38 produced in engineered plant bioreactors were able to mitigate the induction of IL-6, suggesting that their application in the immunotherapy of COVID-19 should be further investigated.

### Materials and Methods

### Design of the Peptides

The peptides were designed by using the homologous sequence of MHV's NSP10 region interacting with NSP16 as this was the aim of inhibition. The HIV-Tat peptide sequence (YGRKKRRQRRRGSG) (SEQ ID NO. 42) was added to the N-terminus. The peptides were modified with N-acetylation and C-amidation, artificially synthesized, purified using High Performance Liquid Chromatography (HPLC) and ensured not to have any disulfide bonds using Mass Spectrometry (MS) (Peptides 2.0 Inc). Prior to use, peptides were dissolved in 1λPBS.

Toxicity Tests of the Peptides for Caco-2 Cells Infected with SARS-COV-2 Virus

Testing for toxicity of the peptides against cells infected with SARS-COV-2 was done in a BSL-3 facility at Utah State University, part of the NIH/NIAID program. Confluent or near-confluent cell culture monolayers of Caco-2 cells were prepared in 96-well disposable microplates the day before testing. Cells were maintained in MEM supplemented with 5% FBS. The peptides were dissolved in 1λPBS and concentrations of 0.1, 1.0, 10, 100, and 200 μg/mL were prepared. Five microwells were used per dilution: three for infected cultures and two for uninfected toxicity cultures. On every plate controls for the experiment consisted of six microwells that were infected but not treated (virus controls) and six that were untreated and uninfected (cell controls). Peptide 1 and Peptide 2 were tested in parallel with a positive control drug using the same method as was applied for the peptides. The positive control was included with every test run. Growth media were removed and the peptides (0.1 mL) were applied to the wells at 2× concentration. Aliquots (0.1 mL), containing virus at ˜60 CCID50 (50% cell culture infectious dose) were added to the wells designated for virus infection. Media devoid of virus was added to the toxicity control wells and cell control wells. Plates were incubated at 37° C. with 5% CO2 until marked CPE (>80% CPE for most virus strains) was observed in virus control wells. The plates were then stained with 0.011% neutral red for two hours at 37° C. in a 5% CO2 incubator. The neutral red medium was removed by complete aspiration, and the cells were rinsed 1× with PBS to remove residual dye. The PBS was completely removed, and the incorporated neutral red was eluted with 50% Sorensen's citrate buffer/50% ethanol for at least 30 minutes. The dye content in each well was quantified using a microplate reader at 540 nm. The dye content in each set of wells was converted to a percentage of dye present in untreated control wells using a Microsoft Excel computer-based spreadsheet and normalized based on the virus control. The 50% effective (EC50, virus-inhibitory) concentrations and 50% cytotoxic (CC50, cell-inhibitory) concentrations were then calculated by regression analysis. It was not possible for us to compute the 50% effective (EC50, virus-inhibitory) since Peptide 1 was too toxic at 11UM while Peptide 2 had no detectable effect against the virus-infected cells even at 200 UM concentration, albeit being non-toxic at that concentration. Production and purification IL-37b and IL-38

Recombinant human cytokines IL-37b and IL-38 (UniProt identifiers Q9NZH6 and Q8WWZ1, respectively) were produced in-planta by Solar Grants Biotechnology Inc. using proprietary methodologies that will be discussed elsewhere due to pending patent applications. Briefly, the cytokines were expressed in their mature forms (V46-D218 for IL-37b, C2-W152 for IL-38) with a C-terminal HIS-tag, purified using immobilized metal-affinity chromatography (IMAC, His SpinTrap Kit, GE Healthcare) and 0.22 μm-filtered (Millipore-Sigma) to obtain sterile solutions. The predicted molecular masses (20.3 kDa for IL-37b; 18.3 kDa for IL-38) were observed following Western blotting with protein-specific antibodies (MyBioSource Inc.). Native tetriary conformation was confirmed for each of the cytokines using protein-specific ELISA tests (R&D Systems Inc.).

### Cell Culture for IL-6 Measurements

The human non-small cell lung cancer cell line NCI-H1792 was obtained from ATCC. Cells were grown in RPMI Medium 1640 (Gibco) supplemented with L-glutamine, 10% fetal bovine serum (Gibco), and 1% penicillin/streptomycin at 37° C. in a humidified atmosphere containing 5% CO2. For cell stimulation experiments, cells (5×104 per well) were seeded into 24-well plates and grown overnight. The next day, the cells were washed with OPTi-MEM (Gibco) and then pre-treated with plant-produced IL-38 (10 uL), IL-37b (10 uL), IL-38 plus IL-37b (5 uL each), or plant-produced GFP (1 μg) in 500 uL OPTi-MEM for 3 hrs at 37° C. PBS (containing 25% glycerol) was added to the controls. Subsequently, peptides (10 μM) were added and incubation was continued for 24 h after which the conditioned media was harvested for determination of IL-6 secretion by ELISA.

### Elisa Assays

Correct folding of plant-produced IL-38 and IL-37b anti-inflammatory cytokines was assessed by enzyme-linked immunosorbent assay (ELISA) using the DuoSet Human IL-38/IL-1F10 kit (R&D systems) and the Human IL-37/IL-1F7 uncoated ELISA kit (Invitrogen), respectively, following the instructions of the manufacturers. Secreted IL-6 was assessed using the Human IL-6 Uncoated ELISA kit (Invitrogen).

### REFERENCES

Akhtar, N., Arida, A. R., and Haqqi, T. M. (2014). The human ribonucleotidyl transferase ZCCHC6 regulates interleukin-6 expression in osteoarthritis chondrocytes. Osteoarthr. Cartil. Allam, G., Gaber. A. M., Othman, S. I., and Abdel-Moneim, A. (2020). The potential role of interleukin-37 in infectious diseases. Int. Rev. Immunol.
Angulo, J., Martinez-Valdebenito, C., Marco, C., Galeno, H., Villagra, E., Vera, L., Lagos, N., Becerra, N., Mora, J., Bermúdez. A., et al. (2017). Serum levels of interleukin-6 are linked to the severity of the disease caused by Andes Virus. PLOS Negl. Trop. Dis.
Ansari, M. Y., Khan, N. M., Ahmad, N., Green, J., Novak, K., and Haqqi, T. M. (2019). Genetic Inactivation of ZCCHC6 Suppresses Interleukin-6 Expression and Reduces the Severity of Experimental Osteoarthritis in Mice. Arthritis Rheumatol.
Bouvet, M., Lugari, A., Posthuma, C. C., Zevenhoven, J. C., Bernard, S., Betzi. S., Imbert, I., Canard, B., Guillemot, J. C., Lécine, P., et al. (2014). Coronavirus Nsp10, a critical cofactor for activation of multiple replicative enzymes. J. Biol. Chem.
Brandão, S. C. S., Ramos, J. de O. X., Dompieri, L. T., Godoi, E. T. A. M., Figueiredo, J. L., Sarinho, E. S. C., Chelvanambi, S., and Aikawa, M. (2020). Is Toll-like receptor 4 involved in the severity of COVID-19 pathology in patients with cardiometabolic comorbidities? Cytokine Growth Factor Rev.
Cagno, V. (2020). SARS-COV-2 cellular tropism. The Lancet Microbe.
Cavalli, G., and Dinarello, C. A. (2018). Suppression of inflammation and acquired immunity by IL-37. Immunol. Rev.
Chen, G., Wu, D., Guo, W., Cao, Y., Huang, D., Wang, H., Wang, T., Zhang, X., Chen, H., Yu, H., et al. (2020). Clinical and immunological features of severe and moderate coronavirus disease 2019. J. Clin. Invest.
Conti, P., Ronconi, G., Caraffa, A., Gallenga, C. E., Ross, R., Frydas, I., and Kritas, S. K. (2020a). Induction of pro-inflammatory cytokines (IL-1 and IL-6) and lung inflammation by Coronavirus-19 (COVI-19 or SARS-COV-2): anti-inflammatory strategies. J Biol Regul Homeost Agents. Conti, P., Caraffa, A., Gallenga, C. E., Ross, R., Kritas, S. K., Frydas, I., Younes, A., and Ronconi, G. (2020b). Coronavirus-19 (SARS-COV-2) induces acute severe lung inflammation via IL-1 causing cytokine storm in COVID-19: a promising inhibitory strategy. J. Biol. Regul. Homeost. Agents.
Cronkite, D. A., and Strutt, T. M. (2018). The regulation of inflammation by innate and adaptive lymphocytes. J. Immunol. Res.
Dienz, O., Rud, J. G., Eaton, S. M., Lanthier, P. A., Burg, E., Drew, A., Bunn, J., Suratt, B. T., Haynes, L., and Rincon, M. (2012). Essential role of IL-6 in protection against H1N1 influenza virus by promoting neutrophil survival in the lung. Mucosal Immunol.
Gao, X., Chan, P. K. S., Lui, G. C. Y., Hui, D. S. C., Chu, I. M. T., Sun, X., Tsang, M. S. M., Chan, B. C. L., Lam. C. W. K., and Wong, C. K. (2021). Interleukin-38 ameliorates poly(I: C) induced lung inflammation: therapeutic implications in respiratory viral infections. Cell Death Dis. Gordon, D., Jang, G., Bouhaddou, M., Xu, J., Obernier, K., O'Meara, M., Guo, J., Swaney, D., Tummino, T., Hüttenhain, R., et al. (2020). A SARS-COV-2-Human Protein-Protein Interaction Map Reveals Drug Targets and Potential Drug-Repurposing. Nature.
Gu, J., Gao, X., Pan, X., Peng, X., Li, Y., and Li, M. (2015). High-level expression and one-step purification of a soluble recombinant human interleukin-37b in Escherichia coli. Protein Expr. Purif. 108, 18-22.
Han, Y., Huard, A., Mora, J., da Silva, P., Brune, B., and Weigert, A. (2020). IL-36 family cytokines in protective versus destructive inflammation. Cell. Signal.
Hu, B., Guo, H., Zhou, P., and Shi, Z. L. (2020). Characteristics of SARS-COV-2 and COVID-19. Nat. Rev. Microbiol.
Ke, M., Chen, Y., Wu, A., Sun. Y., Su, C., Wu, H., Jin, X., Tao, J., Wang, Y., Ma, X., et al. (2012). Short peptides derived from the interaction domain of SARS coronavirus nonstructural protein nsp10 can suppress the 2′-O-methyltransferase activity of nsp10/nsp16 complex. Virus Res.
Krafcikova, P., Silhan, J., Nencka, R., and Boura, E. (2020). Structural analysis of the SARS-COV-2 methyltransferase complex involved in RNA cap creation bound to sinefungin. Nat. Commun.
Li, A., Ling, Y., Song, Z., Cheng, X., Ding, L., Jiang, R., Fu, W., Liu, Y., Hu, H., Yuan, S., et al. (2020a). Correlation Between Early Plasma Interleukin 37 Responses With Low Inflammatory Cytokine Levels and Benign Clinical Outcomes in Severe Acute Respiratory Syndrome Coronavirus 2 Infection. J. Infect. Dis.
Li. J., Guo, M., Tian, X., Liu, C., Wang, X., Yang, X., Wu, P., Xiao, Z., Qu, Y., Yin, Y., et al. (2020b). Virus-host interactome and proteomic survey of PMBCs from COVID-19 patients reveal potential virulence factors influencing SARS-COV-2 pathogenesis. BioRxiv.
Li, J., Guo, M., Tian, X., Wang, X., Yang, X., Wu, P., Liu, C., Xiao, Z., Qu, Y., Yin, Y., et al. (2021). Virus-Host Interactome and Proteomic Survey Reveal Potential Virulence Factors Influencing SARS-COV-2 Pathogenesis. Med.
Lin, H., Ho, A. S., Haley-Vicente, D., Zhang, J., Bernal-Fussell, J., Pace, A. M., Hansen, D., Schweighofer, K., Mize, N. K., and Ford, J. E. (2001). Cloning and Characterization of IL-1HY2, a Novel Interleukin-1 Family Member. J. Biol. Chem.
Liu, T., Zhang, J., Yang, Y., Ma, H., Li, Z., Zhang, J., Cheng, J., Zhang, X., Zhao, Y., Xia, Z., et al. (2020a). The potential role of IL-6 in monitoring severe case of coronavirus disease 2019. MedRxiv.
Liu, Z., VanBlargan, L. A., Rothlauf, P. W., Bloyet, L.-M., Chen, R. E., Stumpf, S., Zhao, H., Errico, J. M., Theel, E. S., Ellebedy, A., et al. (2020b). Landscape Analysis of Escape Variants Identifies SARS-COV-2 Spike Mutations that Attenuate Monoclonal and Serum Antibody Neutralization. SSRN Electron. J.
Mantovani, A., Dinarello, C. A., Molgora, M., and Garlanda, C. (2019). Interleukin-1 and Related Cytokines in the Regulation of Inflammation and Immunity. Immunity.
Masiá, M., Fernández-González, M., Padilla, S., Ortega, P., García, J. A., Agulló, V., Garcia-Abellán, J., Telenti, G., Guillén, L., and Gutiérrez, F. (2020). Impact of interleukin-6 blockade with tocilizumab on SARS-COV-2 viral kinetics and antibody responses in patients with COVID-19: A prospective cohort study. EBioMedicine.
Mauer, J., Denson, J. L., and Brüning, J. C. (2015). Versatile functions for IL-6 in metabolism and cancer. Trends Immunol.
Mei, Y., and Liu, H. (2019). IL-37: An anti-inflammatory cytokine with antitumor functions. Cancer Rep.
Mora, J., Schlemmer, A., Wittig, I., Richter, F., Putyrski, M., Frank, A. C., Han, Y., Jung, M., Ernst, A., Weigert, A., et al. (2016). Interleukin-38 is released from apoptotic cells to limit inflammatory macrophage responses. J. Mol. Cell Biol.
Nold-Petry, C. A. Lo, C. Y., Rudloff, I., Elgass, K. D., Li, S., Gantier, M. P., Lotz-Havla, A. S., Gersting, S. W., Cho, S. X., Lao, J. C., et al. (2015). IL-37 requires the receptors IL-18R and IL-1R8 (SIGIRR) to carry out its multifaceted anti-inflammatory program upon innate signal transduction. Nat. Immunol.
Nold, M. F., Nold-Petry, C. A., Zepp, J. A., Palmer, B. E., Bufler, P., and Dinarello, C. A. (2010). IL-37 is a fundamental inhibitor of innate immunity. Nat. Immunol.
Nyati, K. K., Masuda, K., Mahabub-Uz Zaman, M., Dubey, P. K., Millrine, D., Chalise, J. P., Higa, M., Li, S., Standley, D. M., Saito, K., et al. (2017). TLR4-induced NF-B and MAPK signaling regulate the IL-6 mRNA stabilizing protein Arid5a. Nucleic Acids Res.
Pedersen, S. F., and Ho, Y. C. (2020). SARS-COV-2: A Storm is Raging. J. Clin. Invest.
Scheller, J., Chalaris, A., Schmidt-Arras, D., and Rose-John, S. (2011). The pro- and anti-inflammatory properties of the cytokine interleukin-6. Biochim. Biophys. Acta-Mol. Cell Res. Tang, D., Comish, P., and Kang, R. (2020). The hallmarks of COVID-19 disease. PLOS Pathog. Tian, S., Hu, W., Niu, L., Liu, H., Xu, H., and Xiao, S. Y. (2020). Pulmonary Pathology of Early-Phase 2019 Novel Coronavirus (COVID-19) Pneumonia in Two Patients With Lung Cancer. J. Thorac. Oncol.
Totura, A. L., Whitmore, A., Agnihothram. S., Schäfer, A., Katze, M. G., Heise, M. T., and Baric, R. S. (2015). Toll-like receptor 3 signaling via TRIF contributes to a protective innate immune response to severe acute respiratory syndrome coronavirus infection. MBio.
Ummarino, D. (2017). IL-38 promotes anti-inflammatory effects. Nat. Rev. Rheumatol.
Van De Veerdonk, F. L., Stoeckman, A. K., Wu, G., Boeckermann, A. N., Azam, T., Netea, M. G., Joosten, L. A. B., Van Der Meer, J. W. M., Hao, R., Kalabokis, V., et al. (2012). IL-38 binds to the IL-36 receptor and has biological effects on immune cells similar to IL-36 receptor antagonist. Proc. Natl. Acad. Sci. U.S.A
Velazquez-Salinas, L., Pauszek, S. J., Stenfeldt. C., O'Hearn, E. S., Pacheco, J. M., Borca, M. V., Verdugo-Rodriguez, A., Arzt, J., and Rodriguez, L. L. (2018). Increased virulence of an epidemic strain of vesicular stomatitis virus is associated with interference of the innate response in pigs. Front. Microbiol.
Velazquez-Salinas, L., Verdugo-Rodriguez, A., Rodriguez, L. L., and Borca, M. V. (2019). The role of interleukin 6 during viral infections. Front. Microbiol.
Wang, W., Ye, L., Ye, L., Li, B., Gao, B., Zeng, Y., Kong, L., Fang, X., Zheng, H., Wu, Z., et al. (2007). Up-regulation of IL-6 and TNF-induced by SARS-coronavirus spike protein in murine macrophages via NF-B pathway. Virus Res.
Wang, Y., Van Boxel-Dezaire, A. H. H., Cheon, H., Yang, J., and Stark, G. R. (2013). STAT3 activation in response to IL-6 is prolonged by the binding of IL-6 receptor to EGF receptor. Proc. Natl. Acad. Sci. U.S.A
Wang, Y., Sun, Y., Wu, A., Xu, S., Pan, R., Zeng, C., Jin, X., Ge, X., Shi. Z., Ahola. T., et al. (2015). Coronavirus nsp 10/nsp 16 Methyltransferase Can Be Targeted by nsp 10-Derived Peptide In Vitro and In Vivo To Reduce Replication and Pathogenesis. J. Virol.
WHO (2020). Coronavirus disease (COVID-2019) situation reports. World Heal. Organ.
Xie, L., Huang, Z., Li. H., Liu, X., Zheng, S., and Su, W. (2019). IL-38: A new player in inflammatory autoimmune disorders. Biomolecules.
Xu, W. D., and Huang, A. F. (2018). Role of interleukin-38 in chronic inflammatory diseases: A comprehensive review. Front. Immunol.
Yang, Z., Kang, L., Wang, Y., Xiang, J., Wu, Q., Xu, C., Zhou, Y., Chen, S., Fang, H., Liu, J., et al. (2019). Role of IL-37 in Cardiovascular Disease Inflammation. Can. J. Cardiol.
Yuan, X. L., Li, Y., Pan, X. H., Zhou, M., Gao, Q. Y., and Li, M. C. (2016). Production of recombinant human interleukin-38 and its inhibitory effect on the expression of proinflammatory cytokines in THP-1 cells. Mol. Biol. (Mosk).

### Example 13: Coupling of Plant-Produced Protein a to Agarose Beads and Fabrication of a Chromatography Resin for Purification of Antibodies

### Coupling of Plant-Produced Protein a to Agarose Beads

Protein A was extracted from the leaves of the green bioreactor line described herein and partially purified using Ni-agarose column chromatography. The purified protein A was coupled to agarose beads using the Sulfolink Immobilization kit for proteins (Thermo Fisher, Cat. Nr. #44995), following the instructions of the manufacturer. Briefly, the concentrated, partially purified, protein A solution (450 uL @ 6 mg/mL total protein) was diluted with 500 UL sample preparation buffer (0.1M sodium phosphate, 5 mM EDTA-Na; pH 6.0), reduced in the presence of 50 mM 2-mercaptoethylamine·HCl at 370 C for 90 min, cooled, and added slowly to a Zeba™ Desalt Spin Column that had been equilibrated with coupling buffer (50 mM Tris, 5 mM EDTA-Na; pH 8.5). Next, 100 μL coupling buffer was added to the column, followed by centrifugation at 1,000×g for 1 min at room temperature to collect the desalted protein A. Then, 1 mL of coupling buffer was added to the desalted protein A and the protein was coupled to the SulfoLink Resin (6% crosslinked beaded agarose supplied as 2 mL of a 50% slurry) by incubation on a rocking platform for 15 min at room temperature and then allowed to stand for 30 min at room temperature. Finally, nonspecific binding sites were blocked by the addition of cysteine (50 mM).

Purification of mAb53 Using the Prepared, Protein A-Conjugated Agarose Beads.

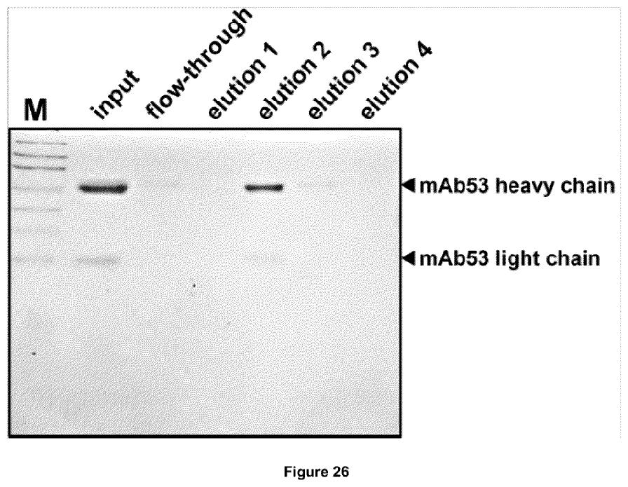

The prepared protein A affinity column was equilibrated at room temperature with 6 mL of phosphate-buffered saline. Next, monoclonal antibody mAb53 (730 μg in 2 mL PBS; Enzo Life Sciences, Cat. Nr. ADI-905-629-100) was allowed to enter the column bed by gravity. Subsequently, 0.2 mL PBS was added, and the binding reaction was allowed to proceed for 60 min at room temperature on a rocking platform. Next, the column was centrifuged at 1000×g for 1 min and the unbound material wash collected. Then, 1 mL PBS was added to the column followed by centrifugation at 1000×g for 1 min. Thereafter, the column was washed 4 times with 2 mL PBS. Finally, the monoclonal antibody was eluted using 8 mL 0.1M glycine (pH 3). Four fractions of 2 mL were collected in tubes already containing 100 μL of 1M Tris.HCl (pH 8.5), FIG. 26.

The above disclosure generally describes the present invention. Although specific terms have been employed herein, such terms are intended in a descriptive sense and not for purposes of limitation.

Patent applications, patents, and publications are cited herein to assist in understanding the embodiments described. All such references cited herein are incorporated herein by reference in their entirety and for all purposes to the same extent as if each individual publication or patent or patent application was specifically and individually indicated to be incorporated by reference in its entirety for all purposes. To the extent publications and patents or patent applications incorporated by reference contradict the disclosure contained in the specification, the specification is intended to supersede and/or take precedence over any such contradictory material.

Although specific embodiments of the invention have been described herein in detail, it will be understood by those skilled in the art that variations may be made thereto without departing from the spirit of the invention or the scope of the appended claims.

It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise than as specifically described without actually departing from the spirit and scope of the present invention as defined by the appended claims.

We claim:

1. A transformation vector for stably transforming a plastid, comprising, an expression cassette, comprising, as operably-linked components, a regulatory sequence operative in the plastid, a heterologous polynucleotide sequence coding for a protein of interest, and, flanking each side of the expression cassette, a first DNA flanking sequence and a second flanking DNA sequence which allow for stable integration of the heterologous polynucleotide sequence coding for the protein of interest into the plastid genome.

2. The vector of claim 1, wherein the first flanking sequence comprises trl, rps12 or trnT.

3. The vector of claim 1 or 2, wherein the second flanking sequence comprise trnA, or trnV or trnG

4. The vector of any one of claims 1 to 3, wherein the first and second flanking sequences are substantially homologous to sequences around an integration site of the plastid genome and provide for homologous recombination to insert the heterologous polynucleotide coding for the protein of interest into the integration site of the plastid genome.

5. The vector of any one of claims 1 to 3, wherein the expression cassette further comprises a spacer region comprising about 50 to about 80 base pairs.

6. The vector of claim 4 or 5, wherein the spacer is from between psbN and psbH genes, or rps2 and atpl genes, or rpoC2 and rps2 genes of the plastid genome.

7. The vector of any one of claims 1 to 6, wherein the regulatory sequence comprises a promoter operative in the plastid genome.

8. The vector of claim 7, wherein the promoter is selected from 16S rRNA, psbA gene or rbcL gene.

9. The vector of claim 7, wherein the promoter is a mutated 16S rRNA promoter with reduced homology to the endogenous 16S rRNA promoter yet with substantially equal functionality.

10. The vector of any one of claims 1 to 9, wherein the regulatory sequence further comprises a 5′ untranslated region (UTR) capable of providing transcription and translation enhancement of the heterologous polynucleotide coding for the protein of interest.

11. The vector of claim 10, wherein the 5′ UTR is a 5′ UTR of psbA.

12. The vector of claim 10, wherein the 5′ UTR is a 5′ UTR of T7G10.

13. The vector of any one of claims 1 to 12, wherein the regulatory sequence further comprises a 3′ UTR capable of conferring stability to a transcript of the protein of interest.

14. The vector of claim 13, wherein the 3′ UTR is a 3′ UTR of psbA.

15. The vector of claim 13, wherein the 3′ UTR is a 3′ UTR of a heterologous psbC gene.

16. The vector of any one of claims 1 to 15, further comprising a DNA sequence coding for a selectable marker.

17. The vector of claim 16, wherein the selectable marker is an antibiotic resistant selectable marker.

18. The vector of claim 17, wherein the antibiotic resistant selectable marker is aadA.

19. The vector of any one of claims 1 to 18, wherein the plastid is selected from a chloroplast, a chromoplast, an amyloplast, a proplastid, a leucoplast or an etioplast.

20. The vector of any one of claims 1 to 18, wherein the plastid is a chloroplast.

21. The vector of any one of claims 1 to 20, wherein the plastid is from a monocot or dicot plant.

22. The vector of claim 21, wherein the dicot plant is a low-nicotine tobacco plant.

23. The vector of any one of claims 1 to 22, wherein the protein of interest is a cytokine.

24. The vector of any one of claims 1 to 22, wherein the protein of interest is IL-38.

25. The vector of any one of claims 1 to 22, wherein the protein of interest is IL-37b.

26. The vector of any one of claims 1 to 22, wherein the protein of interest is IL-33.

27. The vector of any one of claims 1 to 22, wherein the protein of interest is G-CSF.

28. The vector of any one of claims 1 to 22, wherein the protein of interest is Staphylococcus aureus Protein A.

29. The vector of any one of claims 1 to 22, wherein the protein of interest is encoded by a polynucleotide having the sequence defined by SEQ ID NO. 3 or SEQ ID NO. 5.

30. The vector of any one of claims 1 to 22, wherein the protein of interest is at least 70% identical to a protein encoded by a polynucleotide having the sequence defined by SEQ ID NO. 3 or SEQ ID NO. 5 and wherein the protein of interest retains its biological activity.

31. The vector of any one of claims 1 to 22, wherein the protein of interest is expressed in an amount of about 0.1% to about 60% of total soluble protein (TSP).

32. A transformation vector for stably transforming a plastid, comprising, an expression cassette, comprising, as operably-linked components, a promoter comprising psbA operative in the plastid, a heterologous polynucleotide encoding IL-38, and, flanking each side of the expression cassette, a first DNA flanking sequence comprising trnl and a second flanking DNA sequence comprising trnA which allow for stable integration of the heterologous polynucleotide sequence encoding IL-38 into the plastid genome.

33. A transformation vector for stably transforming a plastid, comprising, an expression cassette, comprising, as operably-linked components, a promoter comprising psbA operative in the plastid, a heterologous polynucleotide encoding protein A of Staph aureus, and, flanking each side of the expression cassette, a first DNA flanking sequence comprising trnl and a second flanking DNA sequence comprising tmA which allow for stable integration of the heterologous polynucleotide sequence encoding IL-38 into the plastid genome.

34. The vector of claim 32 or 33, wherein the expression cassette further comprises a spacer region between psbN and psbH genes.

35. The vector of any one of claims 32 to 34, wherein the expression cassette further comprises a 5′UTR comprising T7G10.

36. The vector of any one of claims 32 to 35, wherein the expression cassette further comprises a 3′ UTR comprising psbC.

37. The vector of any one of claims 32 to 36, wherein the expression cassette further comprises a selectable marker comprising aadA.

38. A transformation vector for stably transforming a plastid, comprising, an expression cassette, comprising, as operably-linked components, a promoter comprising psbA operative in the plastid, a heterologous polynucleotide encoding IL-37b, and, flanking each side of the expression cassette, a first DNA flanking sequence comprising trl and a second flanking DNA sequence comprising trnA which allow for stable integration of the heterologous polynucleotide sequence encoding IL-37b into the plastid genome.

39. A transformation vector for stably transforming a plastid, comprising, an expression cassette, comprising, as operably-linked components, a promoter comprising psbA operative in the plastid, a heterologous polynucleotide encoding IL-33, and, flanking each side of the expression cassette, a first DNA flanking sequence comprising trnl and a second flanking DNA sequence comprising trnA which allow for stable integration of the heterologous polynucleotide sequence encoding IL-33 into the plastid genome.

40. A transformation vector for stably transforming a plastid, comprising, an expression cassette, comprising, as operably-linked components, a promoter comprising psbA operative in the plastid, a heterologous polynucleotide encoding G-CSF, and, flanking each side of the expression cassette, a first DNA flanking sequence comprising trnl and a second flanking DNA sequence comprising trnA which allow for stable integration of the heterologous polynucleotide sequence encoding G-CSF into the plastid genome.

41. The vector of any one of claims 38 to 40, wherein the expression cassette further comprises a spacer region between psbN and psbH genes.

42. The vector of any one of claims 38 to 41, wherein the expression cassette further comprises a 5′UTR comprising psbA.

43. The vector of any one of any one of claims 38 to 42, wherein the expression cassette further comprises a 3′ UTR comprising psbC.

44. The vector of any one of claims 38 to 43, wherein the expression cassette further comprises a selectable marker comprising aadA.

45. A plant plastid stably transformed with the transformation vector of any one of claims 1 to 44.

46. A plant cell stably transformed with the transformation vector of any one of claims 1 to 44.

47. A plant stably transformed with the transformation vector of any one of claims 1 to 44.

48. The plant of claim 47, wherein the plant further comprises mature leaves transformed with the vector of any one of claims 1 to 44.

49. The plant of claim 47 or 48, wherein the plant further comprises young leaves transformed with the vector of any one of claims 1 to 44.

50. The plant of any one of claims 47 to 49, wherein the plant further comprises old leaves transformed with the vector of any one of claims 1 to 44.

51. The plant of any one of claims 1 to 50, the protein of interest is present in the plastid in an amount of about 0.1% TSP to about 60% TSP.

52. The plant of any one of claims 1 to 51, the protein of interest is present in the plastid as a monomer, a dimer, a trimer or additional forms of multimers in amount of about 0.1% TSP to about 60% TSP.

53. A progeny of the plant of any one of claims 47 to 52.

54. A seed of the plant of any one of claims 47 to 52.

55. A method for producing a protein of interest comprising: integrating the transformation vector of any one of claims 1 to 44 into a plastid genome of a plant cell; and growing the plant cell to thereby express the protein of interest.

56. The method of claim 55, wherein the plastid genome is from a low-alkaloid tobacco plant.

57. The method of claim 55 or 56, further comprising recovering the protein of interest.

58. The method of claim 57, wherein the recovering comprises isolating and purifying the protein of interest.

59. The method of claim 58, wherein the purifying comprising using an IMAC procedure.

60. The method of any one of claims 55 to 59, wherein the protein of interest is competent to modulate an immune response ex vivo or in vitro.

61. The method of claim 60, wherein the protein of interest dose-dependently modulates the immune response.

62. The method of claim 60 or 61, wherein the immune response comprises modulation of peripheral blood mononuclear cells cytokine secretion in response to inflammatory mediator stimulation.

63. The method of claim 62, wherein the inflammatory mediator comprises LPS or PHA.

64. The method of claim 60 or 61, wherein the immunogenic response comprises modulation of tissue cell cytokine secretion in response to viral stimulation.

65. The method of claim 64, wherein the viral stimulation is SARS-COV-2 stimulation.

66. The method of claim 64 or 65, wherein the tissue cell is a lung cell.

67. Use of the transformation vector of any one of claims 1 to 44 for stably transforming a plant cell.

68. Use of the transformation vector of any one of claims 1 to 44 for stably transforming a plant.

69. Use of the transformation vector of any one of claims 1 to 44 for producing the protein of interest that can modulate an immune response.

70. The use of claim 69, wherein the immune response comprises cytokine secretion from peripheral blood mononuclear cells.

71. The use of claim 69, wherein the immune response comprises cytokine secretion from tissue cells in response to SARS-COV-2 stimulation.

72. A method of treating an inflammatory disorder comprising administering the protein of interest produced from the transformation vector of any one of claims 1 to 44 to a patient in need thereof.

73. Use of the protein of interest produced from the transformation vector of any one of claims 1 to 44 for treating an inflammatory disorder in a patient in need thereof.

74. A composition comprising at least one excipient and the protein of interest produced from the transformation vector of any one of claims 1 to 44.

75. Use of the composition of claim 74 for treating an inflammatory disorder.

76. A protein of interest produced from the transformation vector of any one of claims 1 to 44.

77. A protein of interest produced by the method of any one of claims 55 to 66.

78. Use of the protein of interest of claim 76 or 77, for treating an inflammatory disorder.
